(19) United States
(12) Patent Application Publication
(10) Pub. No.: US 2021/0340568 A1
(43) Pub. Date: Nov. 4, 2021

(54) REGULATED GENE EDITING SYSTEM

(71) Applicant: The University of North Carolina at Chapel Hill, Chapel Hill, NC (US)

(72) Inventor: Richard Jude Samulski, Hillsborough, NC (US)

(21) Appl. No.: 17/283,322

(22) PCT Filed: Oct. 9, 2019
(86) PCT No.: PCT/US2019/055310

§ 371 Date: Apr. 7, 2021

Related U.S. Application Data
(60) Provisional application No. 62/743,317, filed on Oct. 9, 2018.
(60) Provisional application No. 62/870,427, filed on Jul. 3, 2019.

(51) Int. Cl. C12N 15/86; C12N 9/22; C12N 15/113

(52) U.S. Cl. CPC C12N 15/86; C12N 2310/20; C12N 15/113; C12N 9/22

(57) ABSTRACT

The present invention provides a gene editing system having reduced off target effects comprising (a) a vector comprising a nucleic acid sequence encoding a nuclease, wherein the nucleic acid encoding the nuclease contains within its sequence a regulatory nucleic acid sequence having a first and second set of splice elements defining a first and second intron, wherein the first and second intron flank a sequence encoding a non-naturally occurring exon sequence containing an in-frame stop codon sequence, and wherein the first and second intron are spliced from the mRNA message to produce an mRNA encoding a non-functional nuclease that contains an amino acid sequence encoded by the non-naturally occurring exon; and (b) an oligonucleotide that binds to the regulatory sequence. Further provided are methods of using the gene editing system of this invention to regulate transgene expression.

### STATEMENT OF PRIORITY

This application claims the benefit, under 35 U.S.C. § 119(e), of U.S. Provisional Applications No. 62/743,317, filed on Oct. 9, 2018, and No. 62/870,427, filed on Jul. 3, 2019, the entire contents of which are incorporated by reference herein in their entireties.

### STATEMENT REGARDING ELECTRONIC FILING OF A SEQUENCE LISTING

A Sequence Listing in ASCII text format, submitted under 37 C.F.R. § 1.821, entitled 5470-858WO_ST25.txt, 371,885 bytes in size, generated on Oct. 8, 2019 and filed via EFS-Web, is provided in lieu of a paper copy. This Sequence Listing is hereby incorporated herein by reference into the specification for its disclosures.

### FIELD OF THE INVENTION

The present invention relates to compositions and methods of their use for regulated gene editing.

### BACKGROUND OF THE INVENTION

Recent advances in genome sequencing techniques and analysis methods have significantly accelerated the ability to catalog and map genetic factors associated with a diverse range of biological functions and diseases. The ability to precisely target the genome will permit reverse engineering of causal genetic variations by allowing selective alterations of individual genetic elements, as well as to advance synthetic biology, biotechnological, and medical applications. Though advances in genome editing technology have been made, it has been found that a large number of off-target (e.g., unintended mutations) can occur during gene editing, limiting this approach as a therapeutic. Thus, a more precise genome editing system with higher specificity and reliability for its target is desired.

Endogenous gene expression is further regulated at several post-transcriptional levels that might be areas to exploit for more precise control of exogenous gene expression. For example, RNA production is controlled by the rate of transcription, but functional RNA requires correct splicing before the correct gene product can be produced. By regulating splicing of the transgene's RNA, production of the gene product can be controlled. The present invention provides compositions and methods for precisely controlled expression of genome editing systems in a cell, thus reducing off target effects and increasing its specificity.

### SUMMARY OF INVENTION

The present invention provides a system for editing a gene (e.g., altering expression of at least one gene product) having reduced off target effects comprising introducing into a cell having a gene sequence you want to alter (e.g., a target gene sequence) a) a vector (e.g., a viral or non-viral vector, rAAV etc.) comprising a nucleic acid sequence encoding a nuclease, wherein the nucleic acid encoding the nuclease contains within its coding sequence a regulatory nucleic acid sequence having a first and second set of splice elements defining a first and second intron, wherein the first and second intron flank a sequence encoding a non-naturally occurring exon sequence containing an in-frame stop codon sequence, and wherein when the first and second intron are spliced from the pre-mRNA message to produce an mRNA encoding a non-functional nuclease that contains an amino acid sequence encoded by the non-naturally occurring exon; and b) an oligonucleotide that binds to the regulatory sequence, wherein the oligonucleotide prevents splicing of the second set of splice elements from the mRNA within the cell, thereby producing an mRNA that lacks the exon and encodes a nuclease that is functional for gene editing of a target gene. In one embodiment, the system further comprises a gRNA that can bind to the target gene sequence.

In one embodiment of this aspect, the nuclease is a CRISPR-associated nuclease, a meganuclease, a zinc finger nuclease, or a transcription activator like effector nuclease. In one embodiment of this aspect, the nuclease is an endonuclease or an exonuclease.

Any gene can be regulated using the system and methods described herein. For example, in one embodiment the gene to be regulated is a disease associated gene of a disease or disorder selected from the group consisting of: Amyotrophic Lateral Sclerosis; endotoxemia; atherosclerotic vascular disease is coronary artery disease; stent restenosis; carotid metabolic disease; stroke; acute myocardial infarction; heart failure; peripheral arterial disease; limb ischemia; vein graft failure; AV fistula failure; Crohn's disease; ulcerative colitis; ileitis and enteritis; vaginitis; psoriasis and inflammatory dermatoses such as dermatitis; eczema; atopic dermatitis; allergic contact dermatitis; urticaria; vasculitis; spondyloarthropathies; scleroderma; respiratory allergic diseases such as asthma; allergic rhinitis; hypersensitivity lung diseases; arthritis (e.g., rheumatoid and psoriatic); eczema; psoriasis; osteoarthritis; multiple sclerosis; systemic lupus erythematosus; diabetes mellitus; glomerulonephritis; graft rejection (including allograft rejection and graft-v-host disease) or rejection of an engineered tissue; infectious diseases; myositis; inflammatory CNS disorders; stroke; closed-head injuries; neurodegenerative diseases; Alzheimer's disease; encephalitis; meningitis; osteoporosis; gout; hepatitis; hepatic veno-occlusive disease (VOD); hemorrhagic cystitis; nephritis; sepsis; sarcoidosis; conjunctivitis; otitis; chronic obstructive pulmonary disease; sinusitis; Bechet's syndrome; graft-versus-tumor effect; mucositis; appendicitis; ruptured appendix; peritonitis; aortic valve disease; mitral valve disease; Rett's syndrome; tuberous sclerosis; phenylketonuria; Smith-Lemli-Opitz syndrome and fragile X syndrome; Parkinson's disease; Aicardi-Goutières Syndrome; Alexander Disease; Allan-Hemdon-Dudley Syndrome; POLG-Related Disorders; Alpha-Mannosidosis (Type II and III); Alström Syndrome; Angelman Syndrome; Ataxia-Telangiectasia; Neuronal Ceroid-Lipofuscinoses; Beta-Thalassemia; Bilateral Optic Atrophy and (Infantile) Optic Atrophy Type 1; Retinoblastoma (bilateral); Canavan Disease; Cerebrooculofacioskeletal Syndrome 1 [COFS1]; Cerebrotendinous Xanthomatosis; Cornelia de Lange Syndrome; MAPT-Related Disorders; Genetic Prion Diseases; Dravet Syndrome; Early-Onset Familial Alzheimer Disease; Friedreich Ataxia [FRDA]; Fryns Syndrome; Fucosidosis; Fukuyama Congenital Muscular Dystrophy; Galactosialidosis; Gaucher Disease; Organic Acidemias; Hemophagocytic Lymphohistiocytosis; Hutchinson-Gilford Progeria Syndrome; Mucolipidosis II; Infantile Free Sialic Acid Storage Disease; PLA2G6-Associated Neurodegeneration; Jervell and Lange-Nielsen Syndrome; Junctional Epidermolysis Bullosa; Huntington Disease; Krabbe Disease (Infantile); Mitochondrial DNA-Associated Leigh Syndrome and NARP; Lesch-Nyhan Syndrome; LIS1-Associated Lissencephaly; Lowe Syndrome; Maple Syrup Urine Disease; MECP2 Duplication Syndrome; ATP7A-Related Copper Transport Disorders; LAMA2-Related Muscular Dystrophy; Arylsulfatase A Deficiency; Mucopolysaccharidosis Types I; II or III; Peroxisome Biogenesis Disorders; Zellweger Syndrome Spectrum; Neurodegeneration with Brain Iron Accumulation Disorders; Acid Sphingomyelinase Deficiency; Niemann-Pick Disease Type C; Glycine Encephalopathy; ARX-Related Disorders; Urea Cycle Disorders; COL1A1/2-Related Osteogenesis Imperfecta; Mitochondrial DNA Deletion Syndromes; PLP1-Related Disorders; Perry Syndrome; Phelan-McDermid Syndrome; Glycogen Storage Disease Type II (Pompe Disease) (Infantile); MAPT-Related Disorders; MECP2-Related Disorders; Rhizomelic Chondrodysplasia Punctata Type 1; Roberts Syndrome; Sandhoff Disease; Schindler Disease-Type 1; Adenosine Deaminase Deficiency; Smith-Lemli-Opitz Syndrome; Spinal Muscular Atrophy; Infantile-Onset Spinocerebellar Ataxia; Hexosaminidase A Deficiency; Thanatophoric Dysplasia Type 1; Collagen Type VI-Related Disorders; Usher Syndrome Type I; Congenital Muscular Dystrophy; Wolf-Hirschhorm Syndrome; Lysosomal Acid Lipase Deficiency; and Xeroderma Pigmentosum. In one embodiment, the gene being regulated is a gene associated with pain in the peripheral nervous system or the central nervous system.

In one embodiment, the gene being regulated is a dystrophin gene. The dystrophin gene resides on the X chromosome and mutations in the gene can result in various disease states, for example, Duchenne muscular dystrophy, Becker muscular dystrophy, X-linked dilated cardiomyopathy, and familial dilated cardiomyopathy. In one embodiment, the dystrophin gene is targeted at an exon that commonly harbors mutations that result in a disease stated (e.g., 6, 7, 8, 23, 43, 44, 45, 46, 50, 51, 52, 53, or 55).

In one embodiment, a gRNA is present. For example, TGCAAAAACCCAAAATATTT (SEQ ID NO: 81); AAAATATTTTAGCTCCTACT (SEQ ID NO: 82); CAGAGTAACAGTCTGAGTAG (SEQ ID NO: 83); TAAGGGATATTTGTTCTTAC (SEQ ID NO: 84); CTAAGGGATATT TGTT CT TA (SEQ ID NO: 85); and TGTT CT TACAGGCAACAATG (SEQ ID NO: 86). Other exemplary gRNAs are presented herein, for example, in Table 1.

TABLE 1
Sequence of guide RNA for 12 commonly mutated exons of DMD gene

| Exon | gRNA at 5′acceptor site | | SEQ ID | gRNA at 3′ donor site | | SEQ ID |
|---|---|---|---|---|---|---|
| 51 | #1 | TGCAAAAACCCAAAATATTT | 81 | | | |
| | #2 | AAAATATTTTAGCTCCTACT | 82 | | | |
| | #3 | CAGAGTAACAGTCTGAGTAG | 83 | | | |
| 52 | #1 | TAAGGGATATTTGTTCTTAC | 84 | | | |
| | #2 | CTAAGGGATATT TGTT CT TA | 85 | | | |
| | # | TGTT CT TACAGGCAACAATG | 86 | | | |
| 50 | #1 | TGTATGCTTTTCTGTTAAAG | 87 | | | |
| | #2 | AT GT GTAT GC TT TT CT GT TA | 88 | | | |
| | # | GT GTAT GC TT TT CT GT TAAA | 89 | | | |
| 45 | #1 | TT GCCT TT TT GGTATC TTAC | 90 | | | |
| | #2 | TT TGCC TT TT TGGTAT CT TA | 91 | | | |
| | # | CGCTGCCCAATGCCATCCTG | 92 | | | |
| 53 | #1 | ATTTATTTTTCCTTTTATTC | 93 | #4 | AAAGAAAATCACAGAAACCA | 114 |
| | #2 | TTTCCTTTTATTCTAGTTGA | 94 | #5 | AAAAT CACAGAAACCAAGGT | 115 |
| | # | TGATTCTGAATTCTTTCAAC | 95 | #6 | GGTATCTTTGATACTAACCT | 116 |
| 44 | #1 | ATCCATATGCTTTTACCTGC | 96 | | | |
| | #2 | GATCCATATGCTTTTACCTG | 97 | | | |
| | # | CAGATCTGTCAAATCGCCTG | 98 | | | |
| 46 | #1 | TTAT TC TT CT TT CT CCAGGC | 99 | | | |
| | #2 | AATTTTATTCTTCTTTCTCC | 100 | | | |
| | # | CAAT TT TATT CT TC TT TC TC | 101 | | | |
| 43 | #1 | GTTTTAAAATTTTTATATTA | 102 | #4 | TATGTGTTACCTACCCTTGT | 117 |
| | #2 | TTTTATATTACAGAATATAA | 103 | #5 | AAATGTACAAGGACC GACAA | 118 |
| | # | ATATTACAGAATATAAAAGA | 104 | #6 | GTACAAGGACCGACAAGGGT | 119 |
| 7 | #1 | TGTGTATGTGTATGTGTTTT | 105 | | | |
| | #2 | TATGTGTATGTGTTTTAGGC | 106 | | | |
| | # | CTATTCCAGTCAAATAGGTC | 107 | | | |
| 8 | #1 | GTGTAGTGTTAATGTGCTTA | 108 | #4 | T GCAC TATT CT CAACAGGTA | 120 |
| | #2 | GGACTTCTTATCTGGATAGG | 109 | #5 | TCAAATGCACTATTCTCAAC | 121 |
| | # | TAGGTGGTATCAACATCTGT | 110 | #6 | CTTTACACACTTTACCTGTT | 122 |
| 6 | #1 | TGAAAATTTATTTCCACATG | 111 | #4 | ATGCTCTCATCCATAGTCAT | 123 |
| | #2 | GAAAATTTATTTCCACATGT | 112 | #5 | T CT CATCCATAGT CATAGGT | 124 |
| | #3 | TTACATTTTTGACCTACATG | 113 | #6 | CAT CCATAGTCATAGGTAAG | 125 |
| 55 | #1 | TGAACATTTGGTCCTTTGCA | 126 | | | |
| | #2 | TCTGAACATTTGGTCCTTTG | 127 | | | |
| | #3 | TCTCGCTCACTCACCCTGCA | 128 | | | |

In one embodiment, the gene being regulated is a disease or a pain gene. The gene editing system described herein can be used to alter or modulate genes associated with a disease, e.g., Crohn's Disease or neuropathic pain, e.g., pain associated with the peripheral nervous system or the central nervous system. For example, genes that are abnormally expressed (e.g., over expressed, or under expressed) in the dorsal root ganglia of pain patients, or genes that regulate or are required for the function of noxious stimuli transduction; voltage-gated sodium channels (e.g., Ca2+ channels, K+ channels, Na+ channels); NMDA receptors; ligand-gated ion channels; Mas-related G-protein-coupled receptors (Mrgprs); can be repressed using the gene editing system described herein to treat, ameliorate, suppress, or reduce neuropathic pain. Exemplary genes that can be repressed using the gene editing system described herein to treat, ameliorate, suppress, or reduce neuropathic pain include, but are not limited to, Nav1; 1, Nav1.2, Nav1.3, Nav1.4, Nav1.5, Nav1.6, Nav1.7, Nav1.8, and Nav1.9, Angiotensin II Type 2 Receptor, vanilloid receptor-1 (VR-1), tyrosine receptor kinase A (TrkA), bradykinin receptor, CSF1-DAP12 pathway members (e.g., CSF1, CSFR1, or DAP12).

In one embodiment, the system for editing a gene (e.g., altering expression of at least one gene product) associated with neuropathic pain having reduced off target effects comprising introducing into a cell having a target gene sequence a) a vector comprising a nucleic acid sequence encoding a CRISPR-associated nuclease, wherein the nucleic acid encoding the nuclease contains within its sequence a regulatory nucleic acid sequence having a first and second set of splice elements defining a first and second intron, wherein the first and second intron flank a sequence encoding a non-naturally occurring exon sequence containing an in-frame stop codon sequence, and wherein the first and second intron are spliced from the mRNA message to produce an mRNA encoding a non-functional nuclease that contains an amino acid sequence encoded by the non-naturally occurring exon; b) a gRNA that binds to the neuropathic pain-associated gene, e.g., Nav 1.8; and c) an oligonucleotide that binds to the regulatory sequence, wherein within the cell the oligonucleotide prevents splicing of the second set of splice elements from the mRNA, thereby producing an mRNA that lacks the exon and encodes a nuclease that is functional for binding the gRNA and gene editing of the target sequence.

In one embodiment, the gRNA of the described invention is directed to Nav 1.8 for silencing of Nav1.8. Exemplary gRNA that target Nav 1.8 include, but are not limited to gRNAs listed in Table 2.

TABLE 2
Exemplary gRNAs that target Nav1.8 that
can be used with the gene editing
system described herein.

| gRNA targeting Nav 1.8 | SEQ ID NO: |
|---|---|
| GGCACAGCAATAGATCTCCG | 129 |
| TAAGAACTCTGAATGTCCGC | 130 |
| GTTCTTCTGATCAGGTTGAA | 131 |
| TCACGTACCTGAGAGATCCT | 132 |
| GAATAGCCACAGGGCCCGAG | 133 |
| TGAAGCCTTGATAAAGATAC | 134 |

In one embodiment, the gRNA of the described invention is directed to the first 200 bp upstream of the transcription start site (TSS) of Nav 1.8 for activation of Nav1.8. Exemplary gRNA that target Nav 1.8 include, but are not limited to gRNAs listed in Table 3.

TABLE 3
Exemplary gRNAs that activate transcription
of Nav 1.8 that can be used with the gene
editing system described herein.

| gRNA targeting Nav 1.8 | SEQ ID NO: |
|---|---|
| CAGATATGAGGGTGGGAGAA | 135 |
| CAGGGGAATGGGTTCCTGGG | 136 |
| CCCCTCCCTGAACTCACACT | 137 |

In one embodiment of this aspect, and all aspects described herein, the regulatory nucleic acid sequence is a beta-globin mutant intron.

In one embodiment of this aspect, and all aspects described herein, the system comprises at least two regulatory nucleic acid sequences.

In one embodiment of this aspect, and all aspects described herein, the regulatory nucleic acid sequence comprises a sequence selected from the group consisting of: SEQ ID NO: 18 (IVS2-654 intron C-T), SEQ ID NO:50 (IVS2-654 intron with 564CT mutation), SEQ ID NO:51 (IVS2-654 intron with 657G mutation), SEQ ID NO:52 (IVS2-654 intron with 658T mutation), SEQ ID NO:20 (IVS2-654 intron with 657GT mutation), SEQ ID NO:53 (IVS2-654 intron with 200 by deletion), SEQ ID NO:68 (IVS2-654 intron with only 197 bp), SEQ ID NO:55 (IVS2-654 intron with 6A mutation), SEQ ID NO:56 (IVS2-654 intron with 564C mutation), SEQ ID NO:57 (IVS2-654 intron with 841A mutation), SEQ ID NO:59 (IVS2-705 intron with 564CT mutation), SEQ ID NO:60 (IVS2-705 intron with 657G mutation), SEQ ID NO:61 (IVS2-705 intron with 658T mutation), SEQ ID NO:62 (IVS2-705 intron with 657GT mutation), SEQ ID NO:63 (IVS2-705 intron with 200 by deletion), SEQ ID NO:64 (IVS2-705 intron with 425 by deletion), SEQ ID NO:65 (IVS2-705 intron with 6A mutation), SEQ ID NO:66 (IVS2-705 intron with 564C mutation), SEQ ID NO:67 (IVS2-705 intron with 841A mutation). SEQ ID NO: 74, SEQ ID NO:75, SEQ ID NO: 76, SEQ ID NO: 77, SEQ ID NO:78, SEQ ID NO: 143, SEQ ID NO: 144, SEQ ID NO: 145, SEQ ID NO: 146, SEQ ID NO: 147, SEQ ID NO: 148; and in any combination thereof, including singly.

In one embodiment of this aspect, and all aspects described herein, the oligonucleotide that binds to the regulatory sequence comprises a sequence selected from the group consisting of: SEQ ID NO:37 (oligo for IVS2-654 CT), SEQ ID NO:38 (oligo for IVS2-654 with 657GT mutation), SEQ ID NO:39 (oligo for 6A mutation in IVS2-654), SEQ ID NO:40 (oligo for 564C mutation in IVS2-654), SEQ ID NO:41 (oligo for 564CT mutation in IVS2-654), SEQ ID NO:43 (oligo for 841A mutation in IVS2-654), SEQ ID NO:44 (oligo for 657G mutation in IVS2-654), SEQ ID NO:45 (oligo for 658T mutation in IVS2-654), SEQ ID NO:42 (oligo for 705G mutation in IVS2-705). SEQ ID NO:49 (oligo for IVS2-705), SEQ ID NO:76 (Antisense exon 23 skipping inducing oligo) respectively, and SEQ ID NO 138 (Oligo for LUC-AON1), SEQ ID NO: 139 (oligo for LUC-AON2), SEQ ID NO: 140 (Oligo for LUC-AON3), SEQ ID NO: 141 (Oligo for LUC-AON4), SEQ ID NO: 142 (Oligo for IVS2(S0)-654, LUC-654) and SEQ ID NO: 149 (Oligo for WT regulatory).

In one embodiment of this aspect, and all aspects described herein, the oligonucleotide that binds to the regulatory sequence comprises a sequence selected from those listed in Table 4.

TABLE 4
Sequences of the oligonucleotide that binds
to the regulatory sequence described herein.

| | | | Oligo specifically binds |
|---|---|---|---|
| | | Oligo | to Regulatory Sequence |
| | Oligonucleotide Sequence | SEQ ID NO: | of SEQ ID NO: |
| LNA-AON1 | 5′-GtAcTcAcCtGcCcTc-3′ | 138 | 143 |
| LNA-AON2 | 5′-GaAcTtAcCtCgGcAc-3′ | 139 | 144 |
| LNA-AON3 | 5′-GgAcTcAcCtAgTcAg-3′ | 140 | 145 |
| LNA-AON4 | 5′-GcAcTtAcCtAtTgGc-3′ | 141 | 146 |
| LNA-654 | 5′-GcTaTtAcCtTaAcCc-3′ | 142 | 147 |

In one embodiment of this aspect, and all aspects described herein, the oligonucleotide having the sequence of SEQ ID NO: 138 (e.g., LNA-AON1), binds to the regulatory sequence having the sequence of SEQ ID NO: 143.

In one embodiment of this aspect, and all aspects described herein, the oligonucleotide having the sequence of SEQ ID NO: 139 (e.g., LNA-AON2), binds to the regulatory sequence having the sequence of SEQ ID NO: 144.

In one embodiment of this aspect, and all aspects described herein, the oligonucleotide having the sequence of SEQ ID NO: 140 (e.g., LNA-AON3), binds to the regulatory sequence having the sequence of SEQ ID NO: 145.

In one embodiment of this aspect, and all aspects described herein, the oligonucleotide having the sequence of SEQ ID NO: 141 (e.g., LNA-AON4), binds to the regulatory sequence having the sequence of SEQ ID NO: 146.

In one embodiment of this aspect, and all aspects described herein, the oligonucleotide having the sequence of SEQ ID NO: 142 (e.g., LNA-654), binds to the regulatory sequence having the sequence of SEQ ID NO: 147.

In one embodiment of this aspect, and all aspects described herein, the regulatory sequence that the oligonucleotide binds is selected from those listed in Table 5.

TABLE 5
Regulatory sequence that the oligonucleotide binds to.

| | | Oligonucleotide | |
|---|---|---|---|
| | SEQ | that binds the | Oligonucleotide that |
| Regulatory Sequence | ID NO: | regulatory sequence | binds (SEQ ID NO): |
| GAGGGCAG/GTGAGTAC | 143 | LNA-AON1 | 138 |
| GTGCCGAG/GTAAGTTC | 144 | LNA-AON2 | 139 |
| CTGACTAG/GTGAGTCC | 145 | LNA-AON3 | 140 |
| GCCAATAG/GTAAGTGC | 146 | LNA-AON4 | 141 |
| GGGTTAAG/GTAATAGC | 147 | LNA-654 | 142 |

In one embodiment of this aspect, and all aspects described herein, the off-target effects are reduced by at least 30% (by at least 40%, at least 50%, at least 60%, at least 70%, at least 80%, at least 90%.

In one embodiment of this aspect, and all aspects described herein, components (a) and (b) are located on same or different vectors.

In one embodiment of this aspect, and all aspects described herein, component (b) is introduced to the cell as naked DNA. In one embodiment of this aspect, and all aspects described herein, component (b) is introduced to the cell using a lipid formulation. In one embodiment of this aspect, and all aspects described herein, component (b) is introduced to the cell using a nanoparticle.

In one embodiment of this aspect, and all aspects described herein, component (b) is administered at a time point following the administration of (a). In another embodiment of this aspect, and all aspects described herein, components (a) and (b) are administered at substantially the same time.

In one embodiment of this aspect, and all aspects described herein, the expression of (a) is not detected in the cell in the absence of (b), or absence of expression of (b). For example, the expression of (a) is “OFF” in the cell until it is co-expressed in the cell with (b). Following expression of, or presence of (b), (a) is turned “ON” in the cell.

In one embodiment, component (b) controls the “ON” and/or “OFF” status of the gene editing system.

In one embodiment, the gene editing system can be selectively turned “ON” or “OFF”. In another embodiment the gene editing system can be selectively turned “ON” or “OFF” under spatial and/or local control. In one embodiment, the components of the system can delivered/administered locally to a desired site, location, organ, cell type, tissue type, etc., to induce the gene editing system to turn “ON” locally. In one embodiment, the components of the gene editing system can be administered for a given duration to control the timing in which the system is “ON” or “OFF”. It is not required that all components of the system be delivered/administered with spatial and/or temporal control. For example, component (a) can be administered systemically, and component (b) can be administered locally and/or for a specific duration. For example, depending upon a subject's pain, one can turn the system “ON” or “OFF.”

In one embodiment of this aspect, and all aspects described herein, the expression of (a) is dependent on the expression of (b).

In one embodiment of this aspect, and all aspects described herein, the vector is a viral vector. Exemplary viral vectors include, but are not limited to, an AAV vector, an adenovirus vector, a lentivirus vector, a retrovirus vector, a herpesvirus vector, an alphavirus vector, a poxvirus vector a baculovirus vector and a chimeric virus vector.

In one embodiment of this aspect, and all aspects described herein, the vector is a non-viral vector.

In one embodiment of this aspect, and all aspects described herein, the nuclease is a CRISPR-associated nuclease.

In one embodiment of this aspect, and all aspects described herein, the CRISPR-associated nuclease creates double stand breaks for gene editing and wherein the CRISPR-associated nuclease is selected from the group consisting of Cpf1, C2c1, C2c3, Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, C2c1, C2c3, Cas12a, Cas12b, Cas12c, Cas12d, Cas12e, Cas13a, Cas13b, and Cas13c.

In one embodiment of this aspect, and all aspects described herein, the CRISPR-associated nuclease is a Cas9 variant selected from Staphylococcus aureus (SaCas9), Streptococcus thermophilus (StCas9), Neisseria meningitidis (NmCas9), Francisella novicida (FnCas9), and Campylobacter jejuni (CjCas9).

In one embodiment of this aspect, and all aspects described herein, the CRISPR-associated nuclease has been modified for gene-editing without double strand DNA breaks (such as CRISPRi or CRISPRa) and is selected from the group consisting of dCas, nCas, and Cas 13.

In one embodiment of this aspect, and all aspects described herein, the gene editing is decreasing the expression of one or more gene products. In one embodiment of this aspect, and all aspects described herein, the gene editing is increasing expression of one or more gene products.

In one embodiment of this aspect, and all aspects described herein, the CRISPR-associated nuclease is codon optimized for expression in the eukaryotic cell.

In one embodiment of this aspect, and all aspects described herein, the cell is a mammalian or human cell.

In one embodiment of this aspect, and all aspects described herein, the cell is in-vivo or in-vitro.

In one embodiment of this aspect, and all aspects described herein, the target gene is a disease gene.

Another aspect of the invention described herein provides a method for editing a gene in a subject, the method comprising administering any of the systems described herein to a subject in need of gene editing.

### BRIEF DESCRIPTION OF THE DRAWINGS

FIGS. 1A-1C show effect of splice site optimization on induction. (FIG. 1A) Diagram of IVS2-654 Intron and its splicing pattern. Gray boxes: exons of human β-globin, white box: alternatively used exon (AUE), dotted lines: Introns. (FIG. 1B) Modification of splice site. Top: Gray boxes: Luciferase coding region, White box: alternatively used exon (a non-naturally occurring exon of the regulated protein), Solid lines: Intron, Dotted lines: alternative splicing path. Middle: 5′ and 3′ splice site sequences of the IVS2-654 intron. Bottom: Alternative 5′ splice site with modified sequences. (FIG. 1C) Measurement of luciferase activity. We performed luciferase assay 24 hours after transfection of each construct with or without corresponding oligonucleotide that binds the regulatory sequence (AON) into HEK293 cells. The data in the first two rows are indicated relative light unit (RLU)/μg. The data in the third row are presented as the fold increase in expression with AON over expression without AON.

FIGS. 2A-2C show optimization of intron size. (FIG. 2A) Diagram of original IVS2-654 and IVS2 (S0)-654 intron. White box: Alternatively used exon. Dotted lines: introns. Nucleotide numbers of 5′ and 3′ splice site of IVS2 and joining region after deletion for IVS2 (S0) are indicated. (FIG. 2B) Total nucleotide sequences of IVS2 (S0)-654 (SEQ ID NO: 147). (FIG. 2C) Effect of IVS (S0)-654 on induction of luciferase. We performed luciferase assay 24 hours after transfection of each construct with or without AON654 into HEK293 cells. The data are presented as the fold increase in expression with AON654 overexpression without AON654.

FIGS. 3A-3C show regulation of luciferase expression of modified intron containing constructs by their corresponding AONs. (FIG. 3A) Diagram of the constructs and their AON target sequences. (FIG. 3B) Induction of each construct by AONs. Luciferase assay was performed 24 hours after transfection of each construct with or without indicated AONs into HEK293 cells. The data are presented as the fold increase in expression with AONs over expression without AONs. (FIG. 3C) Induction of luciferase expression by corresponding AON.

FIGS. 4A-4B show differential regulation of multiple gene expression by their corresponding AON. (FIG. 4A) Diagram of each construct and their expected pathway by AON. (FIG. 4B) Differential regulation of three individual gene expressions. Top panel shows GFP under fluorescent microscopy. LNADGT1 specifically induced GFP expression. Middle panel shows RFP under fluorescent microscopy. LNADGT2 specifically induced RFP expression. Bottom panel shows measurement of luciferase activity of each sample. LNALucS1 specifically induced luciferase expression.

FIGS. 5A and 5B show regulation of luciferase expression of AAV2.5-CBh-Luc-DGT1 by AON in mouse liver. (FIG. 5A) Luciferase activity for the indicated conditions. (FIG. 5B) Luciferase activity for the indicated conditions, including AON1+I.

FIGS. 6A-6B show regulation of luciferase expression of AAV2.5-CBh-Luc-DGT1 by AON in mouse eyes. (FIG. 6A) An outline of experiment. Short arrowhead refers to time point of vector injection. Arrows refer to time points of AON injection. Long arrowheads refer to time point of luciferase activity measurement. (FIG. 6B) Induction of luciferase expression of vectors by AON. The graph shows luciferase activity (RLU) of mouse eyes after each AON administration.

FIG. 7 shows a schematic of wild-type human β-globin intron splicing. Gray numbered boxes show exons.

FIG. 8 shows a schematic of human β-globin IVS2-654 mutant, which contains point mutation (C to T) at amino acid 654.

FIG. 9 shows a schematic of improper intron splicing of the second intron in the human β-globin IVS2-654 mutant. Improper splicing of intron 2 inhibits β-globin function. Bold arrow represents the preferential splice variant. The 5′ splice site (5′ SS) is labeled.

FIG. 10 shows a schematic of the oligonucleotide that binds the regulatory sequence (visualized by a black bar) that binds the 5′ SS of the human β-globin IVS2-654 mutant and drives the preferential splicing to wild-type splicing.

FIG. 11 shows a schematic of Luc-IVS2-654(B). This construct contains the regulatory sequence that can be alternatively spliced that is presented in FIG. 10 (see corresponding dashed lines), i.e. a first and second set of splice sites defining a first and second intron that flank an exon. This regulatory sequence that can be alternatively spliced is placed in frame into a nucleotide sequence encoding the protein to be regulated, e.g., a reporter gene such as luciferase as exemplified, or a nuclease, such as a CRISPR-associated nuclease. In the absence of an oligo, or the absence of the expression of an oligo, that blocks the second set of splice elements, the insertion of this cassette results in an alternate splicing event that retains the exon that is not naturally occurring in the protein to be regulated (AS) (thin arrow) thereby producing a non-functional protein. When the oligonucleotide that binds the regulatory sequence binds to the cassette, the correct splicing occurs, and that exon is removed (bold arrow) producing a functional protein (CS). Luciferase is exemplified in the Figure. An 11-fold increase in the induction level of luciferase is observed when the oligonucleotide that binds the regulatory sequence that prevents splicing of the second set of splice elements is present.

FIGS. 12A-12C show altered splicing of GFP harboring the IVS2-654(B) cassette. (FIG. 12A) A schematic of GFP654INT that contains that cassette used in FIG. 10 (see corresponding dashed lines) flanking an exon. The oligonucleotide that binds the regulatory sequence is represented by the gray bar. The insertion of this cassette results in an alternate splicing (AS) that retains the exon (bold arrow). When the oligonucleotide that binds the regulatory sequence binds the cassette, the correct splicing (CS) occurs, and that exon is removed (thin arrow). (FIG. 12B) GFP654INT expression in the indicated cell lines with no antisense oligo (ASO), a mismatched oligo (LNA654M), or the oligonucleotide that binds the regulatory sequence (LNA654). Expression of GFP is only visible when then oligonucleotide that binds the regulatory sequence is bound. GFP wtINT is used as a control. (FIG. 12C) Radiograph showing AS or CS in the indicated cell line with no antisense oligo (ASO), a mismatched oligo (LNA654M), or the oligonucleotide that binds the regulatory sequence (LNA654).

FIG. 13 shows in vivo expression of GFP654INT in the eye with no antisense oligo (ASO), a mismatched oligo (LNA654M), or the oligonucleotide that binds the regulatory sequence (LNA654). GFP wtINT is used as a control.

FIG. 14 is a schematic of various pGL3-654 mutants varying the length and number of introns. B is the original 850 bp IVS2-654 intron that contains two sets of splice elements, i.e., four splice sites, an alternative splice site. B(S0) has been altered to reduce the size of the introns while maintaining the splice element sets e.g., deletion of a 200 bp fragment. AB(S0) has two minimal regulatory sequences, each of which bind to an oligonucleotide.

FIGS. 15A-15C show various pGL3-654 mutants that increase the strength of the splice receptor or donor. (FIG. 15A) Schematic of the flanking sequences adjacent to the cassette used in FIG. 10. Mutations to the wild-type sequence (top row) are shown (bottom row). (FIG. 15B) Fold increase for the indicated construct. (FIG. 15C) A schematic of various pGL3-654 mutants with the length and number of introns. Region between slashes is shown in FIG. 15A.

FIG. 16 shows the flanking sequence for the indicated luciferase construct.

FIGS. 17A-17E show the specificity of the given oligonucleotide that binds the regulatory sequence in the indicated mutant. B(S0-GT) (FIG. 17A), LUCS1(e) (FIG. 17B), DGT1(f) (FIG. 17C), DGT2(e) (FIG. 17D), and DGT3(h) (FIG. 17E). Oligonucleotide that binds the regulatory sequence only increase the fold induction when bound to its corresponding mutant.

FIGS. 18A and 18B show in vivo expression of AAT containing the cassette found in FIG. 10. AAT containing the cassette was expressed in the mouse via AAV one year prior to administration of the oligo. (FIG. 18A) Radiograph showing AS or CS of AAT following administration of no antisense oligo (ASO), a mismatched oligo (LNA654M), or the oligonucleotide that binds the regulatory sequence (LNA654). Correct splicing (CS) bottom band. Alternative splicing (AS) top band. (FIG. 18B) ATT expression at the indicated day post induction (e.g., administration of the indicated oligo).

### DETAILED DESCRIPTION OF THE INVENTION

As used herein, “a,” “an” or “the” can be singular or plural, depending on the context of such use. For example, “a cell” can mean a single cell or it can mean a multiplicity of cells.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a composition of this invention, dose, time, temperature, and the like, is meant to encompass variations of ±20%, ±10%, ±5%, ±1%, ±0.5%, or even ±0.1% of the specified amount.

The present invention provides a system for editing a gene (e.g., altering expression of at least one gene product) having reduced off target effects comprising introducing into a cell having a target gene sequence comprising (a) a vector (e.g., a viral or non-viral vector, rAAV etc.) comprising a nucleic acid sequence encoding a nuclease, wherein the nucleic acid encoding the nuclease contains within its sequence a regulatory nucleic acid sequence having a first and second set of splice elements defining a first and second intron, wherein the first and second intron flank a sequence encoding a non-naturally occurring exon sequence containing an in-frame stop codon sequence, and wherein when the first and second intron are spliced from the mRNA message to produce an mRNA encoding a non-functional nuclease that contains an amino acid sequence encoded by the non-naturally occurring exon; and (b) an oligonucleotide that binds to the regulatory sequence, wherein within the cell the oligonucleotide prevents splicing of the second set of splice elements from the mRNA, thereby producing an mRNA that lacks the exon and encodes a nuclease that is functional for binding the gRNA and gene editing of the target sequence.

In one embodiment, components (a) and (b) are located on the same vector. In another embodiment, components (a) and (b) are located on two different vectors.

In one embodiment, the system further comprises introducing a gRNA that binds to the target gene sequence into the cell if the nuclease comprised in the system is a CRISPR-associated nuclease. In one embodiment, components (a) and (b), and the gRNA are located on the same vector. In another embodiment, components (a) and (b), and the gRNA are located on three different vectors. In another embodiment, (a) and (b) are located on the same vector and the gRNA is located on a different vector; or (a) and the gRNA are located on the same vector and (b) is located on a different vector; or (b) and the gRNA are located on the same vector and (a) is located on a different vector. When at least two components described herein are located on the same vector, the order of the component on the vector can be interchanged.

The vector can be, but is not limited to a nonviral vector, a viral vector and a synthetic biological nanoparticle. Nonlimiting examples of a viral vector of this invention include an AAV vector, an adenovirus vector, a lentivirus vector, a retrovirus vector, a herpesvirus vector, an alphavirus vector, a poxvirus vector, a baculovirus vector, and a chimeric virus vector.

In one embodiment, components (a) and (b) are administered to a subject at substantially the same time. In one embodiment, components (a) and (b) are administered to a subject at different time points. For example, component (a) is administered at a later time point than (b). Alternatively, component (a) is administered at an earlier time point than (b). In one embodiment, component (b) is administered at least 1, 2, 3, 4, 5, 6, 7, 8, 9, 10, 11, 12, 13, 14, 15, 16, 17, 18, 19, 20, 21, 22, 23, or more hours after (a); or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more days after (a); or at least 1, 2, 3, 4, 5, 6, 7, 8, 9, 10, 11, or more months after (a); or at least 1, 2, 3, 4, 5, 6, 7, 8, 9, 10, or more years after (a).

In one embodiment, the gRNA is administered at substantially the same time as (a). In another embodiment, the gRNA is administered at a different time point than (a). For example, the gRNA can be administered at a time point prior to administration of (a). Alternatively, the gRNA can be administered at a time point after administration of (a). In one embodiment, the gRNA can be administered at substantially the same time, prior to, or after (b).

In one embodiment, component (b) is administered to a subject once. In an alternative embodiment, component (b) is administered to a subject at least twice, e.g., at least 1, 2, 3, 4, 5, 6, 7, 8, 9, 10, or more times over a given period (e.g., hours, days, months, years, or longer).

In one embodiment, expression of (a) is dependent on the expression of (b). Said another way, (a) will not express in the cell unless (b) is subsequently present within, or expressed in, the same cell. Accordingly, in certain embodiments described herein, the system described herein is introduced (e.g., into a subject) in the OFF position (e.g., not expressed) and contact with an oligonucleotide that binds the regulatory sequence and/or small molecule of this invention switches the system to the ON position (e.g., expressed). Further provided herein are methods of turning a system which is introduced (e.g., into a subject) in the ON position to the OFF position, such as a method for inhibiting production of a heterologous protein and/or RNA that imparts a biological function, comprising: a) contacting an oligonucleotide that binds the regulatory sequence and/or a small molecule with the nucleic acid of this invention under conditions which permit splicing, wherein the small molecule blocks a member of the first set of splice elements, resulting in removal of the second intron, thereby inhibiting production of the first RNA.

The present invention further provides a system for editing a gene (e.g., altering expression of at least one gene product) having reduced off target effects comprising introducing into a cell having a target gene sequence comprising a) a vector (e.g., a viral or non-viral vector, rAAV etc.) comprising a nucleic acid sequence encoding a CRISPR-associated nuclease, wherein the nucleic acid encoding the nuclease contains within its sequence a regulatory nucleic acid sequence having a first and second set of splice elements defining a first and second intron, wherein the first and second intron flank a sequence encoding a non-naturally occurring exon sequence containing an in-frame stop codon sequence, and wherein when the first and second intron are spliced from the mRNA message to produce an mRNA encoding a non-functional nuclease that contains an amino acid sequence encoded by the non-naturally occurring exon; b) a gRNA that binds to the target gene sequence; and c) an oligonucleotide that binds to the regulatory sequence, wherein within the cell the oligonucleotide prevents splicing of the second set of splice elements from the mRNA, thereby producing an mRNA that lacks the exon and encodes a nuclease that is functional for binding the gRNA and gene editing of the target sequence.

In one embodiment, components (a), (b), and (c) are located on the same vector. In another embodiment, components (a), (b), and (c) are located on three different vectors. In another embodiment, (a) and (b) are located on the same vector and (c) is located on a different vector; or (a) and (c) are located on the same vector and (b) is located on a different vector; or (b) and (c) are located on the same vector and (a) is located on a different vector. When at least two components are located on the same vector, the order of the component on the vector can be interchanged.

The vector can be, but is not limited to a nonviral vector, a viral vector and a synthetic biological nanoparticle. Nonlimiting examples of a viral vector of this invention include an AAV vector, an adenovirus vector, a lentivirus vector, a retrovirus vector, a herpesvirus vector, an alphavirus vector, a poxvirus vector, a baculovirus vector, and a chimeric virus vector.

In one embodiment, components (a), (b), and (c) are administered to a subject at substantially the same time. In one embodiment, components (a), (b), and (c) are administered to a subject at different time points. In an alternative embodiment, component (c) is administered to a later time point that (a) and (b), for example component (a) and (b) are administered at substantially the same time, and (c) is administered at least one week after administration. In one embodiment, component (c) is administered at least 1, 2, 3, 4, 5, 6, 7, 8, 9, 10, 11, 12, 13, 14, 15, 16, 17, 18, 19, 20, 21, 22, 23, or more hours after (a) and/or (b); or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more days after (a) and/or (b); or at least 1, 2, 3, 4, 5, 6, 7, 8, 9, 10, 11, or more months after (a) and/or (b); or at least 1, 2, 3, 4, 5, 6, 7, 8, 9, 10, or more years after (a) and/or (b).

In one embodiment, component (c) is administered to a subject once. In an alternative embodiment, component (c) is administered to a subject at least twice, e.g., at least 1, 2, 3, 4, 5, 6, 7, 8, 9, 10, or more times over a given period (e.g., hours, days, months, years, or longer).

In one embodiment, expression of (a) and (b) is dependent on the expression of (c). Said another way, (a) and (b) will not express in the cell unless (c) is subsequently present within, or expressed in, the same cell. Accordingly, in certain embodiments described herein, the system described herein is introduced (e.g., into a subject) in the OFF position (e.g., not expressed) and contact with an oligonucleotide that binds the regulatory sequence and/or small molecule of this invention switches the system to the ON position (e.g., expressed). Further provided herein are methods of turning a system which is introduced (e.g., into a subject) in the ON position to the OFF position, such as a method for inhibiting production of a heterologous protein and/or RNA that imparts a biological function, comprising: a) contacting an oligonucleotide that binds the regulatory sequence and/or a small molecule with the nucleic acid of this invention under conditions which permit splicing, wherein the small molecule blocks a member of the first set of splice elements, resulting in removal of the second intron, thereby inhibiting production of the first RNA.

In one embodiment, the expression of the gRNA is dependent on the expression of (b).

In one embodiment, the nuclease is a CRISPR-associated nuclease, meganuclease, zinc finger nuclease, transcription activator-like effector nuclease, endonuclease, or an exonuclease.

As used herein, the term “nuclease” refers to molecules which possesses activity for DNA cleavage. Particular examples of nuclease agents for use in the methods disclosed herein include RNA-guided CRISPR-Cas9 system, zinc finger proteins, meganucleases, TAL domains, TALENs, yeast assembly recombinases, leucine zippers, CRISPR/Cas endonucleases, and other nucleases known to those in the art. Nucleases can be selected or designed for specificity in cleaving at a given target site. For example, nucleases can be selected for cleavage at a target site that creates overlapping ends between the cleaved polynucleotide and a different polynucleotide. Nucleases having both protein and RNA elements, such as in CRISPR-Cas9, can be supplied with the agents already complexed as a nuclease, or can be supplied with the protein and RNA elements separate, in which case they complex to form a nuclease in the reaction mixtures described herein. In one embodiment, a nuclease other than Cas9 is used.

As used herein, the term “recognition site for a nuclease” refers to a DNA sequence at which a nick or double-strand break is induced by a nuclease. The recognition site for a nuclease can be endogenous (or native) to the cell or the recognition site can be exogenous to the cell. In specific embodiments, the recognition site is exogenous to the cell and thereby is not naturally occurring in the genome of the cell. In still further embodiments, the recognition site is exogenous to the cell and to the polynucleotides of interest that one desires to be positioned at the target locus. In further embodiments, the exogenous or endogenous recognition site is present only once in the genome of the host cell. In specific embodiments, an endogenous or native site that occurs only once within the genome is identified. Such a site can then be used to design nuclease agents that will produce a nick or double-strand break at the endogenous recognition site.

The length of the recognition site can vary, and includes, for example, recognition sites that are about 30-36 bp for a zinc finger nuclease (ZFN) pair (i.e., about 15-18 bp for each ZFN), about 36 bp for a Transcription Activator-Like Effector Nuclease (TALEN), or about 20 bp for a CRISPR/Cas9 guide RNA.

In some embodiments, the recognition site is positioned within the polynucleotide encoding the selection marker. Such a position can be located within the coding region of the selection marker or within the regulatory regions, which influence the expression of the selection marker. Thus, a recognition site of the nuclease agent can be located in an intron of the selection marker, a promoter, an enhancer, a regulatory region, or any non-protein-coding region of the polynucleotide encoding the selection marker. In some embodiments, a nick or double-strand break at the recognition site disrupts the activity of the selection marker. Methods to assay for the presence or absence of a functional selection marker are known to those skilled in the art.

Any nuclease that induces a nick or double-strand break into a desired recognition site can be used in the methods and compositions disclosed herein. A naturally-occurring or native nuclease can be employed so long as the nuclease agent induces a nick or double-strand break in a desired recognition site. Alternatively, a modified or engineered nuclease agent can be employed. An “engineered nuclease” comprises a nuclease that is engineered (modified or derived) from its native form to specifically recognize and induce a nick or double-strand break in the desired recognition site. Thus, an engineered nuclease agent can be derived from a native, naturally-occurring nuclease agent or it can be artificially created or synthesized. The modification of the nuclease agent can be as little as one amino acid in a protein cleavage agent or one nucleotide in a nucleic acid cleavage agent. In some embodiments, the engineered nuclease induces a nick or double-strand break in a recognition site, wherein the recognition site was not a sequence that would have been recognized by a native (non-engineered or non-modified) nuclease agent. Producing a nick or double-strand break in a recognition site or other DNA can be referred to herein as “cutting” or “cleaving” the recognition site or other DNA.

These breaks can then be repaired by the cell in one of two ways: non-homologous end joining and homology-directed repair (homologous recombination). In non-homologous end joining (NHEJ), the double-strand breaks are repaired by direct ligation of the break ends to one another. As such, no new nucleic acid material is inserted into the site, although some nucleic acid material may be lost, resulting in a deletion. In homology-directed repair, a donor polynucleotide with homology to the cleaved target DNA sequence can be used as a template for repair of the cleaved target DNA sequence, resulting in the transfer of genetic information from the donor polynucleotide to the target DNA. Therefore, new nucleic acid material may be inserted/copied into the site. The modifications of the target DNA due to NHEJ and/or homology-directed repair can be used for gene correction, gene replacement, gene tagging, transgene insertion, nucleotide deletion, gene disruption, gene mutation, etc.

In one embodiment, the nuclease is a CRISPR-associated nuclease. The native prokaryotic CRISPR-associated nuclease system comprises an array of short repeats with intervening variable sequences of constant length (i.e., clusters of regularly interspaced short palindromic repeats), and CRISPR-associated (“Cas”) nuclease proteins. The RNA of the transcribed CRISPR array is processed by a subset of the Cas proteins into small guide RNAs, which generally have two components as discussed below. There are at least three different systems: Type I, Type II and Type III. The enzymes involved in the processing of the RNA into mature crRNA are different in the 3 systems. In the native prokaryotic system, the guide RNA (“gRNA”) comprises two short, non-coding RNA species referred to as CRISPR RNA (“crRNA”) and trans-acting RNA (“tracrRNA”). In an exemplary system, the gRNA forms a complex with a nuclease, for example, a Cas nuclease. The gRNA:nuclease complex binds a target polynucleotide sequence having a protospacer adjacent motif (“PAM”) and a protospacer, which is a sequence complementary to a portion of the gRNA. The recognition and binding of the target polynucleotide by the gRNA:nuclease complex induces cleavage of the target polynucleotide. The native CRISPR-associated nuclease system functions as an immune system in prokaryotes, where gRNA:nuclease complexes recognize and silence exogenous genetic elements in a manner analogous to RNAi in eukaryotic organisms, thereby conferring resistance to exogenous genetic elements such as plasmids and phages. It has been demonstrated that a single-guide RNA (“sgRNA”) can replace the complex formed between the naturally-existing crRNA and tracrRNA.

Any CRISPR-associated nuclease can be used in the system and methods of the invention. CRISPR nuclease systems are known to those of skill in the art, e.g., see U.S. Pat. No. 8,993,233, US 2015/0291965, US 2016/0175462, US 2015/0020223, US 2014/0179770, U.S. Pat. Nos. 8,697,359; 8,771,945; 8,795,965; WO 2015/191693; U.S. Pat. No. 8,889,418; WO 2015/089351; WO 2015/089486; WO 2016/028682; WO 2016/049258; WO 2016/094867; WO 2016/094872; WO 2016/094874; WO 2016/112242; US 2016/0153004; US 2015/0056705; US 2016/0090607; US 2016/0029604; U.S. Pat. Nos. 8,865,406; 8,871,445; each of which are incorporated by reference in their entirety.

In one embodiment, the nuclease is a meganuclease. Meganucleases have been classified into four families based on conserved sequence motifs, the families are the LAGLIDADG (SEQ ID NO: 153), GIY-YIG, H-N-H, and His-Cys box families. These motifs participate in the coordination of metal ions and hydrolysis of phosphodiester bonds. HEases are notable for their long recognition sites, and for tolerating some sequence polymorphisms in their DNA substrates. Meganuclease domains, structure and function are known, see for example, Guhan and Muniyappa (2003) Crit Rev Biochem Mol Biol 38:199-248; Lucas et al., (2001) Nucleic Acids Res 29:960-9; Jurica and Stoddard, (1999) Cell Mol Life Sci 55:1304-26; Stoddard, (2006) Q Rev Biophys 38:49-95; and Moure et al., (2002) Nat Struct Biol 9:764. In some examples a naturally occurring variant, and/or engineered derivative meganuclease is used. Methods for modifying the kinetics, cofactor interactions, expression, optimal conditions, and/or recognition site specificity, and screening for activity are known, see for example, Epinat et al., (2003) Nucleic Acids Res 31:2952-62; Chevalier et al., (2002) Mol Cell 10:895-905; Gimble et al., (2003) Mol Biol 334:993-1008; Seligman et al., (2002) Nucleic Acids Res 30:3870-9; Sussman et al., (2004) J Mol Biol 342:31-41; Rosen et al., (2006) Nucleic Acids Res 34:4791-800; Chames et al., (2005) Nucleic Acids Res 33:e178; Smith et al., (2006) Nucleic Acids Res 34:el49; Gruen et al., (2002) Nucleic Acids Res 30:e29; Chen and Zhao, (2005) Nucleic Acids Res 33:el54; WO2005105989; WO2003078619; WO2006097854; WO2006097853; WO2006097784; and WO2004031346, which are incorporated herein by reference in their entireties.

Any meganuclease can be used herein, including, but not limited to, I-Scel, I-Scell, 1-SceIII, I-SceIV, I-SceV, I-SceVI, I-SceVII, I-Ceul, I-CeuAIIP, I-Crel, 1-CrepsbIP, I-CrepsbllP, 1-CrepsbIIIP, 1-CrepsbIVP, I-Tlil, I-Ppol, PI-PspI, F-Scel, F-Scell, F-Suvl, F-TevI, F-TevII, I-Amal, I-Anil, I-Chul, I-Cmoel, I-Cpal, I-CpaII, I-CsmI, I-Cvul, I-CvuAIP, I-Ddil, I-DdiII, I-Dirl, I-Dmol, I-Hmul, I-HmuII, I-HsNIP, I-Llal, I-Msol, I-Naal, I-NanI, I-NcIIP, I-NgrIP, I-Nitl, I-Njal, I-Nsp236IP, I-PakI, I-PboIP, I-PcuIP, I-PcuAI, I-PcuVI, I-PgrlP, I-PobIP, I-Porl, I-PorIIP, I-PbpIP, I-SpBetaIP, I-Scal, I-SexIP, I-SneIP, I-Spoml, I-SpomCP, I-SpomIP, I-SpomIIP, I-SquIP, I-Ssp68O3I, I-SthPhiJP, I-SthPhiST3P, I-SthPhiSTe3bP, I-TdeIP, I-TevI, I-TevII, I-TevIII, I-UarAP, I-UarHGPAIP, I-UarHGPA13P, I-VinIP, I-ZbiIP, PI-MtuI, PI-MtuHIP PI-MtuHIIP, PI-PfuI, PI-PfuII, PI-PkoI, PI-PkoII, PI-Rma43812IP, PI-SpBetaIP, PI-SceI, PI-Tful, PI-TfuII, PI-Thyl, PI-Tlil, PI-TliII, or any active variants or fragments thereof.

In one embodiment, the meganuclease recognizes double-stranded DNA sequences of 12 to 40 base pairs. In one embodiment, the meganuclease recognizes one perfectly matched target sequence in the genome. In one embodiment, the meganuclease is a homing nuclease. In one embodiment, the homing nuclease is a LAGLIDADG (SEQ ID NO: 153) family of homing nuclease. In one embodiment, the LAGLIDADG (SEQ ID NO: 153) family of homing nuclease is selected from I-Scel, I-Crel, and I-Dmol.

In one embodiment, the nuclease is a zinc-finger nuclease (ZFN). In one embodiment, each monomer of the ZFN comprises 3 or more zinc finger-based DNA binding domains, wherein each zinc finger-based DNA binding domain binds to a 3 bp subsite. In other embodiments, the ZFN is a chimeric protein comprising a zinc finger-based DNA binding domain operably linked to an independent nuclease. In one embodiment, the independent endonuclease is a FokI endonuclease. In one embodiment, the nuclease agent comprises a first ZFN and a second ZFN, wherein each of the first ZFN and the second ZFN is operably linked to a FokI nuclease subunit, wherein the first and the second ZFN recognize two contiguous target DNA sequences in each strand of the target DNA sequence separated by about 5-7 bp spacer, and wherein the FokI nuclease subunits dimerize to create an active nuclease that makes a double strand break. See, for example, US20060246567; US20080182332; US20020081614; US20030021776; WO 2002/057308A2; US20130123484; US20100291048; WO 2011/017293A2; and Gaj et al. (2013) Trends in Biotechnology, 31(7):397-405, each of which is herein incorporated by reference in their entireties.

In one embodiment, the nuclease is a Transcription Activator-Like Effector Nuclease (TALEN). TAL effector nucleases are a class of sequence-specific nucleases that can be used to make double-strand breaks at specific target sequences in the genome of a prokaryotic or eukaryotic organism. TAL effector nucleases are created by fusing a native or engineered transcription activator-like (TAL) effector, or functional part thereof, to the catalytic domain of an endonuclease, such as, for example, Fokl. The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See, WO 2010/079430; Morbitzer et al. (2010) PNAS 10.1073/pnas.1013133107; Scholze & Boch (2010) Virulence 1:428-43; Christian et al. Genetics (2010) 186:757-761; Li et al. (2010) Nuc. Acids Res. (2010) doi:10.1093/nar/gkg704; and Miller et al. (2011) Nature Biotechnology 29:143-148; all of which are herein incorporated by reference in their entireties.

Examples of suitable TAL nucleases, and methods for preparing suitable TAL nucleases, are disclosed, e.g., in US Patent Application No. 2011/0239315, 2011/0269234, 2011/0145940, 2003/0232410, 2005/0208489, 2005/0026157, 2005/0064474, 2006/0188987, and 2006/0063231 (each hereby incorporated by reference in their entireties). In various embodiments, TAL effector nucleases are engineered that cut in or near a target nucleic acid sequence in, e.g., a genomic locus of interest, wherein the target nucleic acid sequence is at or near a sequence to be modified by a targeting vector. The TAL nucleases suitable for use with the various methods and compositions provided herein include those that are specifically designed to bind at or near target nucleic acid sequences to be modified by targeting vectors as described herein.

In one embodiment, each monomer of the TALEN comprises 33-35 TAL repeats that recognize a single base pair via two hypervariable residues. In one embodiment, the nuclease agent is a chimeric protein comprising a TAL repeat-based DNA binding domain operably linked to an independent nuclease. In one embodiment, the independent nuclease is a FokI endonuclease. In one embodiment, the nuclease agent comprises a first TAL-repeat-based DNA binding domain and a second TAL-repeat-based DNA binding domain, wherein each of the first and the second TAL-repeat-based DNA binding domain is operably linked to a FokI nuclease subunit, wherein the first and the second TAL-repeat-based DNA binding domain recognize two contiguous target DNA sequences in each strand of the target DNA sequence separated by a spacer sequence of varying length (12-20 bp), and wherein the FokI nuclease subunits dimerize to create an active nuclease that makes a double strand break at a target sequence.

In one embodiment, the nuclease is a ribonuclease that e.g., catalyzes the degradation of RNA. Ribonucleases can be used in concert with other components of the CRISPR-Cas Inspired RNA targeting system (CIRT), e.g., a RNA hairpin-binding protein, a gRNA that interacts with the hairpin-binding protein and the complementary target RNA, and charged protein that binds to and stabilizes the gRNA, for RNA editing purposes. Exemplary ribonucleases include, exoribonucleases (e.g., Polynucleotide Phosphorylase (PNPase), RNase PH, RNase R, RNase D, RNase T, oligoribonuclease, exoribonuclease I, and exoribonuclease II), endoribonucleases (e.g., RNase A, RNase H, RNase III, RNase L, RNase P, RNase PhyM, RNase T1, RNase T2, RNase U2, and RNase V), PIN domain nuclease, inactive PIN domain nuclease, YTHDF1, YTHDF2, hADAR2, mutant hADAR2 (e.g., E488W). Ribonucleases useful for RNA editing with CIRT are further described in, e.g., Rauch, S., et al. Cell; 178 (pg 122-134), 2019; Mali, P. Cell (Leading Edge Previews), 2019; and Lerner, Louise. “Using human genome, scientists build CRISPR for RNA to open pathways for medicine.” 20 Jun. 2019. UChicago News. Web. Accessed 3 Jul. 2019; the contents of which are incorporated herein by reference in their entireties.

In one embodiment, the nuclease is a restriction endonuclease (i.e., restriction enzymes), which include Type I, Type II, Type III, and Type IV endonucleases. Type I and Type III restriction endonucleases recognize specific recognition sites, but typically cleave at a variable position from the nuclease binding site, which can be hundreds of base pairs away from the cleavage site (recognition site). In Type II systems the restriction activity is independent of any methylase activity, and cleavage typically occurs at specific sites within or near to the binding site. Most Type II enzymes cut palindromic sequences, however Type Ila enzymes recognize non-palindromic recognition sites and cleave outside of the recognition site, Type lib enzymes cut sequences twice with both sites outside of the recognition site, and Type Ils enzymes recognize an asymmetric recognition site and cleave on one side and at a defined distance of about 1-20 nucleotides from the recognition site. Type IV restriction enzymes target methylated DNA. Restriction enzymes are further described and classified, for example in the REBASE database (webpage at rebase.neb.com; Roberts et al., (2003) Nucleic Acids Res 31:418-20), Roberts et al., (2003) Nucleic Acids Res 31:1805-12, and Belfort et al., (2002) in Mobile DNA II, pp. 761-783, Eds. Craigie et al., (ASM Press, Washington, D.C.).

In one embodiment, the nuclease is an exonuclease. Exonucleases are enzymes that function by cleaving nucleotides are the end of a polynucleotide chain via a hydrolyzing reaction that breaks phosphodiester binds at either the 5′ or 3′ ends. An exonuclease can be endogenous or exogenous to the cell. Nonlimiting examples of native exonucleases includes exonuclease I, exonuclease II, exonuclease III, exonuclease IV, exonuclease V, and exonuclease VIII.

In another embodiment, the nuclease is Natronobacterium gregoryi Argonaute protein (NgAgo). NgAgo is an endonuclease that utilizes a pair of 5′ phosphorylated, reverse complementary guide DNAs or RNAs (e.g., siRNA) to target and cut a target nucleic acid (e.g., genomic DNA). Importantly, Argonaute proteins do not a requite a motif (e.g., PAM) in the sequence of the target nucleic acid.

Sequences for NgAgo are known in the art. For example, NgAgo can have the sequence of SEQ ID NO: 154.

SEQ ID NO: 154 is an amino acid sequence encoding NgAgo (NCBI accession number: ANC90309.1).

| (SEQ ID NO: 154) | |
|---|---|
| 1 | mtvidldstt tadeltsght ydisvtltgv ydntdeqhpr mslafeqdng erryitlwkn |
| 61 | ttpkdvftyd yatgstyift nidyevkdgy enltatyqtt venataqevg ttdedetfag |
| 121 | gepldhhldd alnetpddae tesdsghvmt sfasrdqlpe wtlhtytlta tdgaktdtey |
| 181 | arrtlaytvr qelytdhdaa pvatdglmll tpeplgetpl dldcgvrvea detrtldytt |
| 241 | akdrllarel veeglkrslw ddylvrgide vlskepvltc defdlheryd lsvevghsgr |
| 301 | aylhinfrhr fvpkltladi dddniypglr vkttyrprrg hivwglrdec atdslntlgn |
| 361 | qsvvayhrnn qtpintdlld aieaadrrvv etrrqghgdd avsfpqella vepnthqikq |
| 421 | fasdgfhqqa rsktrlsasr csekaqafae rldpvrlngs tvefssefft gnneqqlrll |
| 481 | yengesvltf rdgargahpd etfskgivnp pesfevavvl peqqadtcka qwdtmadlln |
| 541 | qagapptrse tvqydafssp esislnvaga idpsevdaaf vvlppdgegf adlasptety |
| 601 | delkkalanm giysqmayfd rfrdakifyt rnvalgllaa aggvaftteh ampgdadmfi |
| 661 | gidvsrsype dgasgqinia atatavykdg tilghsstrp qlgeklqstd vrdimknail |
| 721 | gyqqvtgesp thivihrdgf mnedldpate flneqgveyd iveirkqpqt rllaysdvqy |
| 781 | dtpvksiaai nqnepratva tfgapeylat rdggglprpi qiervagetd ietltrqvyl |
| 841 | lsqshiqvhn starlpitta yadqasthat kgylvqtgaf esnvgfl |

The expression and proper folding of NgAgo can be sensitive to conditions such as salt concentration. NgAgo can be expressed in a cell with a high concentration of salt. NgAgo can be expressed in a cell with a low or moderate salt concentration and the resultant expressed NgAgo protein can be divided into soluble and insoluble fractions. Functional NgAgo can be found in the soluble fraction.

Guide DNA sequences for a target nucleic acid can be any 20-30 base pair (bp) sequence in the target nucleic acid; for example, 22 bp, 24 bp, 26 bp, 28 bp, or 30 bp.

NgAgo comprising the regulatory sequence (beta-globin intron region) is generated as described in Example 1. The regulatory sequence intron region (e.g., SEQ ID NO:53 (IVS2-654 intron with 200 by deletion)) is subcloned into an AAV vector plasmid carrying NgAgo using restriction digestion.

In one embodiment, the nuclease is Artificial restriction DNA cutter (ARCUT). Non-restriction enzyme methodology termed artificial restriction DNA cutter (ARCUT) can be used to edit chromosomal DNA of the cell is using the materials and methods described herein. This method uses pseudo-complementary peptide nucleic acid (pcPNA) to specify the cleavage site within the chromosome or the telomeric region. Once pcPNA specifies the site, excision here is carried out by cerium (CE) and EDTA (chemical mixture), which performs the splicing function. Furthermore, the technology uses a DNA ligase that can later attach any desirable DNA within the spliced site (see e.g., Komiyama M. Chemical modifications of artificial restriction DNA cutter (ARCUT) to promote its in vivo and in vitro applications. Artif. DNA PNA XNA. 2014; 5:e1112457).

In one embodiment the gene to be regulated is a disease associated gene selected from the group consisting of: Amyotrophic Lateral Sclerosis; endotoxemia; atherosclerotic vascular disease is coronary artery disease; stent restenosis; carotid metabolic disease; stroke; acute myocardial infarction; heart failure; peripheral arterial disease; limb ischemia; vein graft failure; AV fistula failure; Crohn's disease; ulcerative colitis; ileitis and enteritis; vaginitis; psoriasis and inflammatory dermatoses such as dermatitis; eczema; atopic dermatitis; allergic contact dermatitis; urticaria; vasculitis; spondyloarthropathies; scleroderma; respiratory allergic diseases such as asthma; allergic rhinitis; hypersensitivity lung diseases; arthritis (e.g., rheumatoid and psoriatic); eczema; psoriasis; osteoarthritis; multiple sclerosis; systemic lupus erythematosus; diabetes mellitus; glomerulonephritis; graft rejection (including allograft rejection and graft-v-host disease) or rejection of an engineered tissue; infectious diseases; myositis; inflammatory CNS disorders; stroke; closed-head injuries; neurodegenerative diseases; Alzheimer's disease; encephalitis; meningitis; osteoporosis; gout; hepatitis; hepatic veno-occlusive disease (VOD); hemorrhagic cystitis; nephritis; sepsis; sarcoidosis; conjunctivitis; otitis; chronic obstructive pulmonary disease; sinusitis; Bechet's syndrome; graft-versus-tumor effect; mucositis; appendicitis; ruptured appendix; peritonitis; aortic valve disease; mitral valve disease; Rett's syndrome; tuberous sclerosis; phenylketonuria; Smith-Lemli-Opitz syndrome and fragile X syndrome; Parkinson's disease; Aicardi-Goutières Syndrome; Alexander Disease; Allan-Hemdon-Dudley Syndrome; POLG-Related Disorders; Alpha-Mannosidosis (Type II and III); Alström Syndrome; Angelman Syndrome; Ataxia-Telangiectasia; Neuronal Ceroid-Lipofuscinoses; Beta-Thalassemia; Bilateral Optic Atrophy and (Infantile) Optic Atrophy Type 1; Retinoblastoma (bilateral); Canavan Disease; Cerebrooculofacioskeletal Syndrome 1 [COFS1]; Cerebrotendinous Xanthomatosis; Cornelia de Lange Syndrome; MAPT-Related Disorders; Genetic Prion Diseases; Dravet Syndrome; Early-Onset Familial Alzheimer Disease; Friedreich Ataxia [FRDA]; Fryns Syndrome; Fucosidosis; Fukuyama Congenital Muscular Dystrophy; Galactosialidosis; Gaucher Disease; Organic Acidemias; Hemophagocytic Lymphohistiocytosis; Hutchinson-Gilford Progeria Syndrome; Mucolipidosis II; Infantile Free Sialic Acid Storage Disease; PLA2G6-Associated Neurodegeneration; Jervell and Lange-Nielsen Syndrome; Junctional Epidermolysis Bullosa; Huntington Disease; Krabbe Disease (Infantile); Mitochondrial DNA-Associated Leigh Syndrome and NARP; Lesch-Nyhan Syndrome; LIS1-Associated Lissencephaly; Lowe Syndrome; Maple Syrup Urine Disease; MECP2 Duplication Syndrome; ATP7A-Related Copper Transport Disorders; LAMA2-Related Muscular Dystrophy; Arylsulfatase A Deficiency; Mucopolysaccharidosis Types I; II or III; Peroxisome Biogenesis Disorders; Zellweger Syndrome Spectrum; Neurodegeneration with Brain Iron Accumulation Disorders; Acid Sphingomyelinase Deficiency; Niemann-Pick Disease Type C; Glycine Encephalopathy; ARX-Related Disorders; Urea Cycle Disorders; COL1A1/2-Related Osteogenesis Imperfecta; Mitochondrial DNA Deletion Syndromes; PLP1-Related Disorders; Perry Syndrome; Phelan-McDermid Syndrome; Glycogen Storage Disease Type II (Pompe Disease) (Infantile); MAPT-Related Disorders; MECP2-Related Disorders; Rhizomelic Chondrodysplasia Punctata Type 1; Roberts Syndrome; Sandhoff Disease; Schindler Disease-Type 1; Adenosine Deaminase Deficiency; Smith-Lemli-Opitz Syndrome; Spinal Muscular Atrophy; Infantile-Onset Spinocerebellar Ataxia; Hexosaminidase A Deficiency; Thanatophoric Dysplasia Type 1; Collagen Type VI-Related Disorders; Usher Syndrome Type I; Congenital Muscular Dystrophy; Wolf-Hirschhorn Syndrome; Lysosomal Acid Lipase Deficiency; and Xeroderma Pigmentosum.

In one embodiment, the gene being regulated is a dystrophin gene. The dystrophin gene resides on the X chromosome and mutations in the gene can result in various disease states, for example, Duchenne muscular dystrophy, Becker muscular dystrophy, X-linked dilated cardiomyopathy, and familial dilated cardiomyopathy. In one embodiment, the dystrophin gene is targeted at an exon that commonly harbors mutations that result in a disease stated (e.g., 6, 7, 8, 23, 43, 44, 45, 46, 50, 51, 52, 53, or 55).

Exemplary guide RNA (gRNA) to DMD include, but are not limited, to gRNA listed in Table 1.

Methods for targeting the DMD gene for its silencing are further described in, e.g., International Patent Applications WO 2016/025469 and WO 2016/161380, which are incorporated herein by reference in their entireties.

In one embodiment, the gene being regulated is a UBE3A. UBE3A is biallelically expressed in certain tissues, for example, neurons express only maternally-inherited copies of UBE3A. Inactivating or deleterious mutations of maternal UBE3A gene in a neuron, which resides in chromosome 15q1-q13, results in Angelman Syndrome. In one embodiment, neuronal UBE3A is regulated. In one embodiment, paternal UBE3A, which is imprinted, i.e., silenced, in neuronal cells, is regulated. Modulation of UBE3A for the treatment of Angelman Syndrome is further described in, e.g., Huang, H S., et al. Nature; Vol. 481, 2012; Judson, M C., et al. Neuron; Vol. 90, 2016; and Judson, M C., et al. Trends in Neurosciences; 34(6), 2011; the contents of which are incorporated herein by reference in their entireties.

In another embodiment, the gene being regulated is a disease gene selected from the group consisting of 1p36; 18p; 6p21.3; 14q32; AAAS; FGD1; EDNRB; CP (3p26.3); LMBR1; COL2A1 (12q13.11); 4p16.3; HMBS; ADSL; ABCD1; JAG1; NOTCH2; TP63; TREX1; RNASEH2A; RNASEH2B; RNASEH2C; SAMHD1; ADAR; IFIH1; GFAP; HGD; 10q26.13; ATP1A3; ALMS1; ALAD; FGFR2; VPS33B; ATM; PITX2; FOXO1A; FOXC1; PAX6; 10q26; FGFR2; IGF-2; CDKN1C; H19; KCNQ1OT1; BTD; BCS1L; 15q26.1; 17 FLCN; ATP2A1; MAOA; NOTCH3; HTRA1; X 17q24.3-q25.1; ASPA; RAB23; SNAP29; FTR (7q31.2); PMP22; MFN2; CHD7; LYST; RUNX2; ERCC6; ERCC8; X RPS6KA3; COH1; COL11A1; COL11A2; COL2A1; NTRK1; PTEN; CPOX; 14q13-q21; 5p; 16q12; FGFR2; FGFR3; FGFR3; ATP2A2; Xp11.22 CLCN5; OCRL; WT1; 18q; 22q11.2; HSPB8; HSPB1; HSPB3; GARS; REEP1; IGHMBP2; SLC5A7; DCTN1; TRPV4; SIGMAR1; COL1A1; COL1A2; COL3A1; COL5A1; COL5A2; TNXB; ADAMTS2; PLOD1; B4GALT7; DSE; EMD; LMNA; SYNE1; SYNE2; FHL1; TMEM43; FECH; FANCA; FANCB; FANCC; FANCD1; FANCD2; FANCE; FANCF; FANCG; FANCI; FANCJ; FANCL; FANCM; FANCN; FANCP; FANCS; RAD51C; XPF; GLA (Xq22.1); APC; IKBKAP; MYCN; MED12; FXN; GALT; GALK1; GALE; GBA (1); PAX6; GCDH; ETFA; ETFB; ETFDH; BCS1L; MYO5A; RAB27A; MLPH; ATP2C1 (3); ABCA12; HFE; HAMP; HFE2B; TFR2; TF; CP; FVIII; UROD; 3q12; ENG; ACVRL1; MADH4; GNE; MYHC2A; VCP; HNRPA2B1; HNRNPA1; EXT1; EXT2; EXT3; HPS1; HPS3; HPS4; HPS5; HPS6; HPS7; AP3B1; PMP22; NODAL; NKX2-5; ZIC3; CCDC11; CFC1; SESN1; CBS (gene); HD; IDS; IDUA; AASS; AGXT; GRHPR; DHDPSL; ABCA1; COL2A1; FGFR3 (4p16.3); 20q11.2; IKBKG (Xq28); TBX4; 15q11-14; FGFR2; INPP5E; TMEM216; AHI1; NPHP1; CEP290; TMEM67; RPGRIP1L; ARL13B; CC2D2A; OFD1; TMEM138; TCTN3; ZNF423; AMRC9; ALS2; COL2A1; PDGFRB; GAL; ATP13A2; LCAT; HPRT (X); TP53; MSH2; MLH1; MSH6; PMS2; PMS1; TGFBR2; MLH3; RYR1 (19q13.2); BCKDHA; BCKDHB; DBT; DLD; ARSB; 20 q13.2-13.3; XK (X); AP1S1; MEFV; ATP7A (Xq21.1); MMAA; MMAB; MMACHC; MMADHC; LMBRD1; MUT; RAB3GAP (2q21.3); ASPM (1q31); GALNS; GLB1; ZEB2 (2); FGFR3; MEN1; RET; MSTN; DMPK; CNBP; HYAL1; 17q11.2; SMPD1; NPA; NPB; NPC1; NPC2; GLDC; AMT; GCSH; PTPN11; KRAS; SOS1; RAFI; NRAS; HRAS; BRAF; SHOC2; MAP2K1; MAP2K2; CBL; RELN; RAG1; RAG2; COL1A1; COL1A2; IFITM5; PANK2 (20p13-p12.3); UROD; PDS; STK11; FGFR1; FGFR2; PAH; AASDHPPT; TCF4 (18); PKD1 (16) or PKD2 (4); DNAI1; DNAH5; TXNDC3; DNAH11; DNAI2; KTU; RSPH4A; RSPH9; LRRC50; PROC; PROS1; ABCC6; RP1; RP2; RPGR; PRPH2; IMPDH1; PRPF31; CRB1; PRPF8; TULP1; CA4; HPRPF3; ABCA4; EYS; CERKL; FSCN2; TOPORS; SNRNP200; PRCD; NR2E3; MERTK; USH2A; PROM1; KLHL7; CNGB1; TTC8; ARL6; DHDDS; BEST1; LRAT; SPARA7; CRX; MECP2; ESCO2; CREBBP; HEXB; SGSH; NAGLU; HGSNAT; GNS; HSPG2; COL2A1; FBN1; 11p15; Xp11.22; PHF8; ABCB7; SLC25A38; GLRX5; GUSB; DHCR7; 17p11.2; ATXN1; ATXN2; ATXN3; PLEKHG4; SPTBN2; CACNA1A; ATXN7; ATXN8OS; ATXN10; TTBK2; PPP2R2B; KCNC3; PRKCG; ITPR1; TBP; KCND3; FGF14; FGFR3; ABCA4; CNGB3; ELOVL4; PROM1; COL11A1; COL11A2; COL2A1; COL9A1; COL2A1; HEXA (15); GCH1; PCBD1; PTS; QDPR; MTHFR; DHFR; FGFR3; 5q32-q33.1 (TCOF1; POLR1C; or POLR1D); TSC1; TSC2; MYO7A; USH1C; CDH23; PCDH15; USH1G; USH2A; GPR98; DFNB31; CLRN1; PPOX; VHL; PAX3; MITF; WS2B; WS2C; SNAI2; EDNRB; EDN3; SOX10; COL11A2; ATP7B; C20RF37 (2q22.3-q35); 4p16.3; 15 ERCC4; CENPVL1; CENPVL2; GSPT2; MAGED1; ALAS2 (X); PEX1; PEX2; PEX3; PEX5; PEX6; PEX10; PEX12; PEX13; PEX14; PEX16; PEX19; and PEX26.

In one embodiment, the gene being regulated is a gene associated with neuropathic pain. Neuropathic pain is characterized by a spontaneous hypersensitive pain response and can typically persist long after the original nerve injury has healed. This unusually heightened pain response can be observed as hyperalgesia (an increased sensitivity to a noxious pain stimulus) or allodynia (an abnormal pain response to a non-noxious stimulus, e.g., cold, warmth, or touch). Neuropathic pain can be acute or chronic. Exemplary types of neuropathic pain include postherpetic neuralgia, HIV-distal sensory polyneuropathy, diabetic neuropathic pain, neuropathic pain associated with traumatic nerve injury, neuropathic pain associated with stroke, neuropathic pain associated with multiple sclerosis, neuropathic pain associated with syringomyelia, neuropathic pain associated with epilepsy, neuropathic pain associated with spinal cord injury, and neuropathic pain associated with cancer.

The gene editing system described herein can be used to alter or modulate genes associated with neuropathic pain, e.g., pain associated with the peripheral nervous system or the central nervous system. For example, genes that are abnormally expressed (e.g., over expressed, or under expressed) in the dorsal root ganglia of pain patients, or genes that regulate or are required for the function of noxious stimuli transduction; voltage-gated sodium channels (e.g., Ca2+ channels, K+ channels, Na+ channels); NMDA receptors; ligand-gated ion channels; Mas-related G-protein-coupled receptors (Mrgprs); can be repressed using the gene editing system described herein to treat, ameliorate, suppress, or reduce neuropathic pain. Exemplary genes that can be repressed using the gene editing system described herein to treat, ameliorate, suppress, or reduce neuropathic pain include, but are not limited to, Nav1.1, Nav1.2, Nav1.3, Nav1.4, Nav1.5, Nav1.6, Nav1.7, Nav1.8, and Nav1.9, Angiotensin II Type 2 Receptor, vanilloid receptor-1 (VR-1), tyrosine receptor kinase A (TrkA), bradykinin receptor, CSF1-DAP12 pathway members (e.g., CSF1, CSFR1, or DAP12).

In one embodiment, the system for editing a gene (e.g., altering expression of at least one gene product) associated with neuropathic pain having reduced off target effects comprising introducing into a cell having a target gene sequence (a) a vector comprising a nucleic acid sequence encoding a CRISPR-associated nuclease, wherein the nucleic acid encoding the nuclease contains within its sequence a regulatory nucleic acid sequence having a first and second set of splice elements defining a first and second intron, wherein the first and second intron flank a sequence encoding a non-naturally occurring exon sequence containing an in-frame stop codon sequence, and wherein the first and second intron are spliced from the mRNA message to produce an mRNA encoding a non-functional nuclease that contains an amino acid sequence encoded by the non-naturally occurring exon; (b) a gRNA that binds to the neuropathic pain-associated gene, e.g., Nav 1.8; and (c) an oligonucleotide that binds to the regulatory sequence, wherein within the cell the oligonucleotide prevents splicing of the second set of splice elements from the mRNA, thereby producing an mRNA that lacks the exon and encodes a nuclease that is functional for binding the gRNA and gene editing of the target sequence.

In one embodiment, the gRNA is directed to Nav 1.8. Exemplary gRNA that target Nav 1.8 for inhibition include, but are not limited to gRNAs listed in Table 2.

In certain embodiments, the CRISPR-associated nuclease, for example, used to modulate pain genes is linked to a function domain that promotes repression of a gene (e.g., an overexpressed disease gene), resulting in repressed transcription of the gene. Exemplary functional domains for fusing with a DNA-binding domain such as, for example, a deadCas9, to be used for repressing expression of a gene, e.g., Nav 1.8, is a KOX repression domain or a KRAB repression domain from the human KOX-1 protein (see, e.g., Thiesen et al., New Biologist 2, 363-374 (1990); Margolin et al., Proc. Natl. Acad. Sci. USA 91, 4509-4513 (1994); Pengue et al., Nucl. Acids Res. 22:2908-2914 (1994); Witzgall et al., Proc. Natl. Acad. Sci. USA 91, 4514-4518 (1994). Another suitable repression domain is methyl binding domain protein 2B (MBD-2B) (see, also Hendrich et al. (1999) Mamm Genome 10:906¬912 for description of MBD proteins). Another exemplary repression domain is that associated with the v-ErbA protein. See, for example, Damm, et al. (1989) Nature 339:593-597; Evans (1989) Int. J. Cancer Suppl. 4:26-28; Pain et al. (1990) New Biol. 2:284-294; Sap et al. (1989) Nature 340:242-244; Zenke et al, (1988) Cell 52:107-119; and Zenke et al. (1990) Cell 61:1035-1049. Additional exemplary repression domains include, but are not limited to, KRAB (also referred to as “KOX”), SID, MBD2, MBD3, members of the DNMT family (e.g., DNMT1, DNMT3A, DNMT3B), Rb, and MeCP2. See, for example, Bird et al. (1999) Cell 99:451-454; Tyler et al. (1999) Cell 99:443-446; Knoepfler et al. (1999) Cell 99:447-450; and Robertson et al. (2000) Nature Genet. 25:338-342. Additional exemplary repression domains include, but are not limited to, ROM2 and AtHD2A. See, for example, Chem et al. (1996) Plant Cell 8:305-321; and Wu et al. (2000) Plant J. 22:19-27.

In one embodiment, the CRISPR-associated nuclease of the described invention, for example, deadCas9, is linked to a KOX repression domain.

In certain embodiments, the CRISPR-associated nuclease, for example, used to modulate a disease-associated gene or pain genes is linked to a function domain that promotes transcriptional activation of a gene (e.g., an under expressed disease gene), resulting in activated transcription of the gene. Suitable domains for achieving such activation include the HSV VP16 activation domain (see, e.g., Hagmann et al., J. Virol. 71, 5952-5962 (1997)) nuclear hormone receptors (see, e.g., Torchia et al., Curr. Opin. Cell. Biol. 10:373-383 (1998)); the p65 subunit of nuclear factor kappa B (Bitko & Barik, J. Virol. 72:5610-5618 (1998) and Doyle & Hunt, Neuroreport 8:2937-2942 (1997)); Liu et al., Cancer Gene Ther. 5:3-28 (1998)), or artificial chimeric functional domains such as VP64 (Seifpal et al., EMBO J. 11, 4961-4968 (1992)). Additional exemplary activation domains include, but are not limited to, VP16, VP64, p300, CBP, PCAF, SRC1 PvALF, AtHD2A and ERF-2. See, for example, Robyr et al. (2000) Mol. Endocrinol. 14:329-347; Collingwood et al. (1999) J. Mol. Endocrinol. 23:255-275; Leo et al. (2000) Gene 245:1-11; Manteuffel-Cymborowska (1999) Acta Biochim. Pol. 46:77-89; McKenna et al. (1999) J. Steroid Biochem. Mol. Biol. 69:3-12; Malik et al. (2000) Trends Biochem. Sci. 25:277-283; and Lemon et al. (1999) Curr. Opin. Genet. Dev. 9:499-504; OsGAI, HALF-1, Cl, AP1, ARF-5, -6, -7, and -8, CPRF1, CPRF4, MYC-RP/GP, and TRABI. See, for example, Ogawa et al. (2000) Gene 245:21-29; Okanami et al. (1996) Genes Cells 1:87-99; Goff et al. (1991) Genes Dev. 5:298-309; Cho et al. (1999) Plant Mol. Biol. 40:419-429; Ulmason et al. (1999) Proc. Natl. Acad. Sci. USA 96:5844-5849; Sprenger-Haus-sels et al. (2000) Plant J. 22:1-8; Gong et al. (1999) Plant Mol. Biol. 41:33-44; and Hobo et al. (1999) Proc. Natl. Acad. Sci. USA 96:15,348-15,353.

In one embodiment, the gene editing system described herein is used to activate transcription of a repressed gene. For example, the system described herein can be used to activate transcription of a gene described herein (e.g., a disease gene or gene associate with pain (e.g., repressed Nav 1.8).

In one embodiment, the gRNA is directed to the first 200 bp upstream of the transcription start site (TSS) of Nav 1.8 and results in robust transcriptional activation. Exemplary gRNA that target Nav 1.8 for transcriptional activation include, but are not limited to gRNAs listed in Table 3.

The regulatory sequence in embodiments of the invention can be a nucleotide sequence that defines an intron that comprises one or more mutations, the presence of which results in a first set of splice elements and a second set of splice elements. In some embodiments, the regulatory sequence can be a sequence that defines an intron-exon-intron region, wherein a mutation in either the intron and/or exon region results in the presence of a first set of splice elements and a second set of splice elements. In this latter embodiment, when the second set of splice elements is active, the result is production of an RNA comprising the exon of the intron-exon-intron region.

Screening methods are also provided herein, such as a method of identifying oligonucleotides or other compounds or complexes that block a member of the second set of splice elements of the regulatory nucleic acid of the gene editing system described herein, comprising: (a) contacting within a cell, a nucleic acid encoding the nuclease comprising the regulatory nucleic acid sequence (or alternatively reporter gene comprising the regulatory nucleic acid) with the oligo/compound under conditions that permit splicing; and b) detecting the production of mRNA lacking the non-naturally occurring exon sequence within the regulatory nucleic acid sequence, whereby the production such mRNA identifies a oligo or compound/complex that blocks a member of the second set of splice elements. Alternatively, detection of functional protein, for example reporter protein, or nuclease is the indicator of an oligo/compound that inhibits/blocks the second set of splice elements.

An intron is a portion of eukaryotic DNA or RNA that intervenes between the coding portions, or “exons,” of that DNA or RNA. Introns and exons are transcribed from DNA into RNA termed “primary transcript, precursor to RNA” (or “pre-mRNA”). Introns must be removed from the pre-mRNA so that the protein encoded by the exons can be produced. The removal of introns from pre-mRNA and subsequent joining of the exons is carried out in the splicing process.

The splicing process is a series of reactions that are carried out on RNA after transcription (i.e., post-transcriptionally) but before translation and that are mediated by splicing factors. Thus, a “pre-mRNA” is an RNA that contains both exons and one or more introns, and a “messenger RNA (mRNA or RNA)” is an RNA from which any introns have been removed and wherein the exons are joined together sequentially so that the gene product can be produced therefrom, either by translation with ribosomes into a functional protein or by translation into a functional RNA.

Introns are characterized by a set of “splice elements” that are part of the splicing machinery and are required for splicing. Introns are relatively short, conserved nucleic acid segments that bind the various splicing factors that carry out the splicing reactions. Thus, each intron is defined by a 5′ splice site, a 3′ splice site, and a branch point situated there between. Splice elements also comprise exon splicing enhancers and silencers, situated in exons, as well as intron splicing enhancers and silencers situated in introns at a distance from the splice sites and branch points. In addition to splice site and branch points, these elements control alternative, aberrant and constitutive splicing.

Various promoters that direct expression of the nuclease comprising the regulatory sequence can be used in the gene editing system described herein. Examples include, but are not limited to, constitutive promoters, repressible promoters, and/or inducible promoters, some nonlimiting examples of which include viral promoters (e.g., CMV, SV40), tissue specific promoters (e.g., muscle (e.g., MCK), heart (e.g., NSE), eye (e.g., MSK) and synthetic promoters (SP1 elements) and the chicken beta actin promoter (CB or CBA). The promoter can be present in any position on where it is in operable association with the nuclease sequence.

In addition, one or more promoters, which can be the same or different, can be present in the same nucleic acid molecule, either together or positioned at different locations on the nucleic acid molecule relative to one another and/or relative to a nuclease sequence and/or a regulatory sequence present within the nucleic acid. Furthermore, an internal ribosome entry signal (IRES) and/or other ribosome-readthrough element can be present on the nucleic acid molecule. One or more such IRESs and/or ribosome readthrough elements, which can be the same or different, can be present in the same nucleic acid molecule, either together and/or at different locations on the nucleic acid molecule. Such IRESs and ribosome readthrough elements can be used to translate messenger RNA sequences via cap-independent mechanisms when multiple nuclease sequences are present on a nucleic acid molecule.

The regulatory sequence is found within the coding region of the nuclease and is placed such that when the exon of the regulatory sequence is expressed, it has an in frame stop codon. As exemplified herein below, the regulatory sequence can be included anywhere within the coding region of the nuclease, for example, Cpf1 or Cas9, or other nuclease. In some embodiments, the regulatory sequence is positioned anywhere within the 5′ one/third of the nucleotides of the nuclease sequence, anywhere within the middle one/third of the nucleotides of the nuclease sequence, and/or anywhere within the 3′ one/third of the nucleotides of the nuclease sequence. In some embodiments, the regulatory sequence is positioned anywhere between an open reading frame and a poly(A) site in the nuclease sequence. Preferably, the regulatory sequence is positioned at or near the 5′end of the nuclease coding sequence, for example, within 5, 10, 15, 20, 25, 30, 35, 40, 45, 50, 60, 70, 80, 90, 100, 125, 150, 175, 200, 250, 300, 350, 400, 450, 500, 550, 600, 650, 700, 750, 800, 850, 900 or 1000 nucleotides from the 5′ end. The regulatory nucleic acid is positioned anywhere within the nucleic acid sequence that encodes the nuclease such that the exon that is non-naturally occurring in the protein is expressed having an in-frame stop codon.

In certain embodiments wherein two or more regulatory sequences are present in the gene editing system of this invention, the two or more regulatory sequences can be positioned to be separated by at least about 5, 10, 15, 20, 25, 30, 35, 40, 45, 50, 60, 70, 80, 90, 100, 125, 150, 175, 200, 250, 300, 350, 400, 450, 500, 550, 600, 650, 700, 750, 800, 850, 900 or 1000 nucleotides, including any number of nucleotides between 5 and 1000 not specifically recited herein.

The regulatory sequence of the nucleic acid molecule of this invention can comprise, consist essentially of and/or consist of a first and second set of splice elements defining a first and second intron sequences that flank a non-naturally occurring exon. “A non-naturally occurring exon” as used herein, is an exon that is not normally present in the wild-type protein to be regulated, and its presence in the coding sequence results in expression of a protein lacking wild type function. When the first and second intron sequence are spliced individually a RNA molecule that encodes a non-functional nuclease is produced, e.g., because it comprises the non-naturally occurring exon having a stop codon. Alternatively, in the absence of activity at a second set of splice elements the exon and first and second intron are all spliced to produce an mRNA encoding a nuclease functional for gene editing, e.g., base editing or endonuclease activity for gene replacement/repair. In some embodiments, the regulatory sequence of this invention can comprise one or more mutations, which can be a substitution, addition, deletion, etc.

The components of the gene editing system can be present in a vector and such a vector can be present in a cell. Any suitable vector is encompassed in the embodiments of this invention, including, but not limited to, nonviral vectors (e.g., nucleic acids, minicircles, linear DNA, plasmids, poloxymers, exosomes, and liposomes), viral vectors and synthetic biological nanoparticles (BNP) (e.g., synthetically designed from different adeno-associated viruses, as well as other parvoviruses).

It is apparent to those skilled in the art that any suitable vector can be used to deliver the gene editing system of this invention. The choice of delivery vector can be made based on a number of factors known in the art, including age and species of the target host, in vitro vs. in vivo delivery, level and persistence of expression desired, intended purpose (e.g., for therapy or polypeptide production), the target cell or organ, route of delivery, size of the isolated nucleic acid, safety concerns, and the like.

Suitable vectors also include virus vectors (e.g., retrovirus, alphavirus; vaccinia virus; adenovirus, adeno-associated virus, or herpes simplex virus), lipid vectors, poly-lysine vectors, synthetic polyamino polymer vectors that are used with nucleic acid molecules, such as plasmids, and the like.

Any viral vector that is known in the art can be used in the present invention. Examples of such viral vectors include, but are not limited to vectors derived from: Adenoviridae; Birnaviridae; Bunyaviridae; Caliciviridae, Capillovirus group; Carlavirus group; Carmovirus virus group; Group Caulimovirus; Closterovirus Group; Commelina yellow mottle virus group; Comovirus virus group; Coronaviridae; PM2 phage group; Corcicoviridae; Group Cryptic virus; group Cryptovirus; Cucumovirus virus group Family ([PHgr]6 phage group; Cysioviridae; Group Carnation ringspot; Dianthovirus virus group; Group Broad bean wilt; Fabavirus virus group; Filoviridae; Flaviviridae; Furovirus group; Group Germinivirus; Group Giardiavirus; Hepadnaviridae; Herpesviridae; Hordeivirus virus group; Illarvirus virus group; Inoviridae; Iridoviridae; Leviviridae; Lipothrixviridae; Luteovirus group; Marafivirus virus group; Maize chlorotic dwarf virus group; icroviridae; Myoviridae; Necrovirus group; Nepovirus virus group; Nodaviridae; Orthomyxoviridae; Papovaviridae; Paramyxoviridae; Parsnip yellow fleck virus group; Partitiviridae; Parvoviridae; Peaenation mosaic virus group; Phycodnaviridae; Picornaviridae; Plasmaviridae; Prodoviridae; Polydnaviridae; Potexvirus group; Potyvirus; Poxviridae; Reoviridae; Retroviridae; Rhabdoviridae; Group Rhizidiovirus; Siphoviridae; Sobemovirus group; SSV 1-Type Phages; Tectiviridae; Tenuivirus; Tetraviridae; Group Tobamovirus; Group Tobravirus; Togaviridae; Group Tombusvirus; Group Torovirus; Totiviridae; Group Tymovirus; and Plant virus satellites.

Protocols for producing recombinant viral vectors and for using viral vectors for nucleic acid delivery can be found, e.g., in Current Protocols in Molecular Biology, Ausubel, F. M. et al. (eds.) Greene Publishing Associates, (1989) and other standard laboratory manuals (e.g., Vectors for Gene Therapy. In: Current Protocols in Human Genetics. John Wiley and Sons, Inc.: 1997). Nonlimiting examples of vectors employed in the methods of this invention include any nucleotide construct used to deliver nucleic acid into cells, e.g., a plasmid, a nonviral vector or a viral vector, such as a retroviral vector which can package a recombinant retroviral genome (see e.g., Pastan et al., Proc. Natl. Acad. Sci. U.S.A. 85:4486 (1988); Miller et al., Mol. Cell. Biol. 6:2895 (1986)). For example, the recombinant retrovirus can then be used to infect and thereby, deliver a nucleic acid of the invention to the infected cells. The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naldini et al., Science 272:263-267, 1996), pseudotyped retroviral vectors (Agrawal et al., Exper. Hematol. 24:738-747, 1996), and any other vector system now known or later identified. Also included are chimeric viral particles, which are well known in the art and which can comprise viral proteins and/or nucleic acids from two or more different viruses in any combination to produce a functional viral vector. Chimeric viral particles of this invention can also comprise amino acid and/or nucleotide sequence of non-viral origin (e.g., to facilitate targeting of vectors to specific cells or tissues and/or to induce a specific immune response). The present invention also provides “targeted” virus particles (e.g., a parvovirus vector comprising a parvovirus capsid and a recombinant AAV genome, wherein an exogenous targeting sequence has been inserted or substituted into the parvovirus capsid).

Physical transduction techniques can also be used, such as liposome delivery and receptor-mediated and other endocytosis mechanisms (see, for example, Schwartzenberger et al., Blood 87:472-478, 1996). This invention can be used in conjunction with any of these and/or other commonly used nucleic acid transfer methods. Appropriate means for transfection, including viral vectors, chemical transfectants, or physico-mechanical methods such as electroporation and direct diffusion of DNA, are described by, for example, Wolff et al., Science 247:1465-1468, (1990); and Wolff, Nature 352:815-818, (1991).

Thus, administration of the gene editing system of this invention can be achieved by any one of numerous, well-known approaches, for example, but not limited to, direct transfer of the nucleic acids, in a plasmid or viral vector, or via transfer in cells or in combination with carriers such as cationic liposomes. Such methods are well known in the art and readily adaptable for use in the methods described herein. Furthermore, these methods can be used to target certain diseases and tissues, organs and/or cell types and/or populations by using the targeting characteristics of the carrier, which would be well known to the skilled artisan. It would also be well understood that cell and tissue specific promoters can be employed in the gene editing system of this invention to target specific tissues and cells and/or to treat specific diseases and disorders.

A cell comprising the gene editing system of this invention can be any cell including but not limited to cells from muscle (e.g., smooth muscle, skeletal muscle, cardiac muscle myocytes), liver (e.g., hepatocytes), heart, brain (e.g., neurons), eye (e.g., retinal; corneal), pancreas, kidney, endothelium, epithelium, stein cells (e.g., bone marrow; cord blood), tissue culture cells (e.g., HeLa cells), etc., as are well known in the art.

In one embodiment, the gene editing systems described herein reduces off-target effects (e.g., caused by, for example, CRISPR/Cas gene editing such as Cas3 or Cas9, or TALEN gene editing) by at least 5%, 10%, 15%, 20%, 25%, 30%, 35%, 40%, 45%, 50%, 55%, 60%, 65%, 70%, 75%, 80%, 85%, 90%, 95%, 99%, or more, as compared to the off-target effects of a given engineered gene editing system (e.g., CRISPR/Cas, TALEN, Zinc Finger) that does not have the components of the claimed invention. As used herein, an “off target effect” refers to a nonspecific, or unintended, genetic mutation that arises through the use of an engineered nuclease activity, for example an endonuclease of the gene editing system. A nuclease that is not bound to its target DNA can cleave off-target double stranded breaks and create a genetic mutation at this location. An “off target effect” can be an unintended point mutation, deletion, insertion, inversion, translocation, etc. One skilled in the art can determine if an off target effect has occurred via, e.g., genome sequencing before and after activation of the gene editing system described herein to determine if genetic mutations are present, for example, at locations other than the target sequence following gene editing. Methods for assessing off-target effects follow gene editing are further reviewed in, e.g., Patent App. No.: WO 2015/113063; Slaymaker, et al. Science, 2016; 351(6268): 84-88; Morgens, et al. Nature Communications. 2017; 8(15178); Koo, et al, Mol Cells. 205: 38(6): 475-481; and Haeussler, et al. Genome Biology. 2016; 17:148; each of which are incorporated herein by reference in their entireties.

In some embodiments, the nucleic acids of the present invention have a reduced level of “leakiness” when compared with other gene editing systems. By “leakiness” is meant an amount of gene product or functional RNA that is produced when the system is in the “OFF” position. For example, in some embodiments described herein, the present system is in the “OFF” position when the gene editing system of this invention has no contact with an oligonucleotide that binds the regulatory sequence, small molecule and/or other compound of this invention and thus, the first intron is not being spliced. Leakiness can be a problem inherent in such regulatory systems but the level of leakiness can be less in some embodiments of the present system than in systems known in the art. Thus, the present invention also provides a gene expression regulation system having reduced leakiness in comparison with other gene expression regulation systems, wherein the system comprises the gene editing system of this invention and/or a vector of this invention. The degree to which leakiness is reduced in the present system in comparison to other systems can be 5, 10, 15, 20, 25, 30, 35, 40, 45, 50, 55, 60, 65, 70, 75, 80, 85, 90, 95, or 100% less than the amount of leakiness observed in art-known systems.

As one example, the amount of leakiness of a system can be determined by employing a reporter gene in the system and detecting the amount of reporter gene product produced when the system is in the “OFF” position. Any number of assays can be employed to detect reporter gene product, including but not limited to, protein detection assays such as ELISA and Western blotting and nucleic acid detection assays such as polymerase chain reaction, Southern blotting and Northern blotting. Other assays for detection of gene product can include functional assays, e.g., measurement of an amount of biological activity attributed to the gene product. The nucleic acids and methods of the present invention can be employed in comparative assays to demonstrate a reduced level of leakiness in comparison to other known gene regulation expression systems and nucleic acids employed therein.

Further provided herein are various methods of using the gene editing system of this invention. In one embodiment, a method for editing a gene is provided. The method comprises administering to a cell the following three components of the gene editing system i) a vector comprising a nucleic acid sequence encoding a nuclease, wherein the nucleic acid encoding the nuclease contains within its sequence a regulatory nucleic acid sequence having a first and second set of splice elements defining a first and second intron, wherein the first and second intron flank a sequence encoding a non-naturally occurring exon sequence containing an in-frame stop codon sequence, and wherein the first and second intron are spliced from the mRNA message to produce an mRNA encoding a non-functional nuclease that contains an amino acid sequence encoded by the non-naturally occurring exon; and ii) an oligonucleotide that binds to the regulatory sequence, wherein within the cell the oligonucleotide prevents splicing of the second set of splice elements from the pre-mRNA, thereby producing an mRNA that lacks the exon and encodes a nuclease that is functional for binding the gRNA and gene editing of the target sequence.

In one embodiment, the method further comprises administering a gRNA to the cell if the nuclease used in the system is a CRISPR-associated nuclease.

In one embodiment, the nuclease is a CRISPR-associated nuclease, for example a Cas protein. Exemplary Cas proteins include, but are not limited to, Cpf1, C2c1, C2c3, Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, C2c1, C2c3, Cas12a, Cas12b, Cas12c, Cas12d, Cas12e, Cas13a, Cas13b, and Cas13c.

In one embodiment the CRISPR-associated nuclease is Cas9 or Cas9 variant, e.g., isolated from the bacterium Streptococcus pyogenes (SpCas9). The CRISPR-associate nuclease associates with guide RNA (gRNA) that guides the nuclease to the desired target sequence, e.g., having a protospacer adjacent motif (PAM) sequence, downstream of the target sequence for its cutting action. Once Cas9 recognizes the PAM sequence (5′-NGG-3 in case of SpCas9, where N is any nucleotide), it creates a double-strand break (DSB) at the target locus. Cas9 activity is a collective effort of two parts of the protein: the recognition lobe that senses the complementary sequence of gRNA and the nuclease lobe that cleaves the DNA.

In one embodiment, the CRISPR-associated nuclease is an enhanced specificity spCas9 (eSpCas9) variant, eSpCas9 variants are further described in Slaymaker, et al. Science. 2016; 351(6268): 84-88, which is incorporated herein by reference in its entirety.

In one embodiment the CRISPR-associated nuclease is a natural variant of Cas. Cas9 Variants include e.g., Staphylococcus aureus (SaCas9), Streptococcus thermophilus (StCas9), Neisseria meningitidis, Francisella novicida (FnCas9), and Campylobacter jejuni (CjCas9), to name a few, in CRISPR experiments. The nuclease can be determined based on preferred PAM sequence or size. For example, in one embodiment, the nuclease is a SaCas9 nuclease, which is about 1 kb smaller in size than SpCas9 so it can be packaged into viral vectors more easily and e.g., are two of the most compact naturally occurring CRISPR variants. SaCas9 is further described in, e.g., CasX and CasY (Burstein, David, et al. New CRISPR-Cas systems from uncultivated microbes. Nature 542.7640 (2017): 237; Ran, F. A., et al. In vivo genome editing using Staphylococcus aureus Cas9. Nature 520(186); 2015; and Friedland, A E. Characterization of Staphylococcus aureus Cas9: a smaller Cas9 for all-in-one adeno-associated virus delivery and paired nickase application. Genome Biol. 16:257; 2015; the contents of which are incorporated herein by reference in their entireties.

Sequences for Cas9 for various species are known in the art. For example, S. aureus Cas9 (saCas9) has the sequence of SEQ ID NO: 150.

SEQ ID NO: 150 is an amino acid sequence encoding S. aureus Cas9.

(SEQ ID NO: 150)
MKRNYILGLD IGITSVGYGI IDYETRDVID AGVRLFKEAN
VENNEGRRSK RGARRLKRRR RHRIQRVKKL LFDYNLLTDH
SELSGINPYE ARVKGLSQKL SEEEFSAALL HLAKRRGVHN
VNEVEEDTGN ELSTKEQISR NSKALEEKYV AELQLERLKK
DGEVRGSINR FKTSDYVKEA KQLLKVQKAY HQLDQSFIDT
YIDLLETRRT YYEGPGEGSP FGWKDIKEWY EMLMGHCTYF
PEELRSVKYA YNADLYNALN DLNNLVITRD ENEKLEYYEK
FQIIENVFKQ KKKPTLKQIA KEILVNEEDI KGYRVTSTGK
PEFTNLKVYH DIKDITARKE IIENAELLDQ IAKILTIYQS
SEDIQEELTN LNSELTQEEI EQISNLKGYT GTHNLSLKAI
NLILDELWHT NDNQIAIFNR LKLVPKKVDL SQQKEIPTTL
VDDFILSPVV KRSFIQSIKV INAIIKKYGL PNDIIIELAR
EKNSKDAQKM INEMQKRNRQ TNERIEEIIR TTGKENAKYL
IEKIKLHDMQ EGKCLYSLEA IPLEDLLNNP FNYEVDHIIP
RSVSFDNSFN NKVLVKQEEN SKKGNRTPFQ YLSSSDSKIS
YETFKKHILN LAKGKGRISK TKKEYLLEER DINRFSVQKD
FINRNLVDTR YATRGLMNLL RSYFRVNNLD VKVKSINGGF
TSFLRRKWKF KKERNKGYKH HAEDALIIAN ADFIFKEWKK
LDKAKKVMEN QMFEEKQAES MPEIETEQEY KEIFITPHQI
KHIKDFKDYK YSHRVDKKPN RELINDTLYS TRKDDKGNTL
IVNNLNGLYD KDNDKLKKLI NKSPEKLLMY HHDPQTYQKL
KLIMEQYGDE KNPLYKYYEE TGNYLTKYSK KDNGPVIKKI
KYYGNKLNAH LDITDDYPNS RNKVVKLSLK PYRFDVYLDN
GVYKFVTVKN LDVIKKENYY EVNSKCYEEA KKLKKISNQA
EFIASFYNND LIKINGELYR VIGVNNDLLN RIEVNMIDIT
YREYLENMND KRPPRIIKTI ASKTQSIKKY STDILGNLYE
VKSKKHPQII KKG

In one embodiment, the CRISPR-associated nuclease is a Cas 9 derived from Campylobacter jejuni (C. jejuni). This C. jejuni Cas9 (CjCas9) is further described in, e.g., International patent application WO 2016/021973A1, which is incorporated herein by reference in its entirety.

SEQ ID NO: 152 is an amino acid sequence encoding CjCas9.

```
(SEQ ID NO: 152)
MARILAFDIG ISSIGWAFSE NDELKDCGVR IFTKVENPKT
                                      60 70 80
GESLALPRRL ARSARKRLAR RKARLNHLKH LIANEFKLNY
                90 100 110 120
EDYQSFDESL AKAYKGSLIS PYELRFRALN ELLSKQDFAR
              130 140 150 160
VILHIAKRRG YDDIKNSDDK EKGAILKAIK QNEEKLANYQ
              170 180 190 200
SVGEYLYKEY FQKFKENSKE FTNVRNKKES YERCIAQSFL
              210 220 230 240
KDELKLIFKK QREFGFSFSK KFEEEVLSVA FYKRALKDFS
              250 260 270 280
HLVGNCSFFT DEKRAPKNSP LAFMFVALTR IINLLNNLKN
              290 300 310 320
TEGILYTKDD LNALLNEVLK NGTLTYKQTK KLLGLSDDYE
              330 340 350 360
FKGEKGTYFI EFKKYKEFIK ALGEHNLSQD DLNEIAKDIT
              370 380 390 400
LIKDEIKLKK ALAKYDLNQN QIDSLSKLEF KDHLNISFKA
              410 420 430 440
LKLVTPLMLE GKKYDEACNE LNLKVAINED KKDFLPAFNE
              450 460 470 480
TYYKDEVTNP VVLRAIKEYR KVLNALLKKY GKVHKINIEL
              490 500 510 520
AREVGKNHSQ RAKIEKEQNE NYKAKKDAEL ECEKLGLKIN
              530 540 550 560
SKNILKLRLF KEQKEFCAYS GEKIKISDLQ DEKMLEIDHI
              570 580 590 600
YPYSRSFDDS YMNKVLVFTK QNQEKLNQTP FEAFGNDSAK
              610 620 630 640
WQKIEVLAKN LPTKKQKRIL DKNYKDKEQK NFKDRNLNDT
              650 660 670 680
RYIARLVLNY TKDYLDFLPL SDDENTKLND TQKGSKVHVE
              690 700 710 720
AKSGMLTSAL RHTWGFSAKD RNNHLHHAID AVIIAYANNS
              730 740 750 760
IVKAFSDFKK EQESNSAELY AKKISELDYK NKRKFFEPFS
              770 780 790 800
GFRQKVLDKI DEIFVSKPER KKPSGALHEE TFRKEEEFYQ
              810 820 830 840
SYGGKEGVLK ALELGKIRKV NGKIVKNGDM FRVDIFKHKK
              850 860 870 880
TNKFYAVPIY TMDFALKVLP NKAVARSKKG EIKDWILMDE
              890 900 910 920
NYEFCFSLYK DSLILIQTKD MQEPEFVYYN AFTSSTVSLI
              930 940 950 960
VSKHDNKFET LSKNQKILFK NANEKEVIAK SIGIQNLKVF
              970 980
EKYIVSALGE VTKAEFRQRE DFKK
```

In one embodiment the CRISPR-associated nuclease is Cas12a (also known as Cpf1). As Cas9 requires guanine-rich PAM sequence of NGG, it is not well suited for targeting AT-rich sequences. Zetsche et al. characterized a nuclease (see e.g., US Patent Application US 2016/0208243 for sequence and variants, incorporated by reference in its entirety), CRISPR from Prevotella and Francisella 1 (Cfp1; now classified as Cas12a) that can be used when targeting AT-rich DNA sequences. Cfp1 creates a staggered double-stranded cut, rather than blunt-end cut generated by SpCas9, in the target DNA, and is useful for experiments relying on the HDR repair outcome. Also, Cfp1 is smaller than SpCas9 and does not require a tracer RNA. The guide RNA required by Cfp1 is therefore shorter in length, making it more economical to produce.

Sequences for Cfp1 for various species are known in the art. For example, Acidaminococcus sp. Cfp1 has the sequence of SEQ ID NO: 151.

SEQ ID NO: 151 is an amino acid sequence encoding Acidaminococcus sp. Cfp1.

(SEQ ID NO: 151)
MTQFEGFTNLYQVSKTLRFELIPQGKTLKHIQEQGFIEEDKARNDHYKELK
PIIDRIYKTYADQCLQLVQLDWENLSAAIDSYRKEKTEETRNALIEEQATY
RNAIHDYFIGRTDNLTDAINKRHAEIYKGLFKAELFNGKVLKQLGTVTTTE
HENALLRSFDKFTTYFSGFYENRKNVFSAEDISTAIPHRIVQDNFPKFKEN
CHIFTRLITAVPSLREHFENVKKAIGIFVSTSIEEVFSFPFYNQLLTQTQI
DLYNQLLGGISREAGTEKIKGLNEVLNLAIQKNDETAHIIASLPHRFIPLF
KQILSDRNTLSFILEEFKSDEEVIQSFCKYKTLLRNENVLETAEALFNELN
SIDLTHIFISHKKLETISSALCDHWDTLRNALYERRISELTGKITKSAKEK
VQRSLKHEDINLQEIISAAGKELSEAFKQKTSEILSHAHAALDQPLPTTLK
KQEEKEILKSQLDSLLGLYHLLDWFAVDESNEVDPEFSARLTGIKLEMEPS
LSFYNKARNYATKKPYSVEKFKLNFQMPTLASGWDVNKEKNNGAILFVKNG
LYYLGIMPKQKGRYKALSFEPTEKTSEGFDKMYYDYFPDAAKMIPKCSTQL
KAVTAHFQTHTTPILLSNNFIEPLEITKEIYDLNNPEKEPKKFQTAYAKKT
GDQKGYREALCKWIDFTRDFLSKYTKTTSIDLSSLRPSSQYKDLGEYYAEL
NPLLYHISFQRIAEKEIMDAVETGKLYLFQIYNKDFAKGHHGKPNLHTLYW
TGLFSPENLAKTSIKLNGQAELFYRPKSRMKRMAHRLGEKMLNKKLKDQKT
PIPDTLYQELYDYVNHRLSHDLSDEARALLPNVITKEVSHEIIKDRRFTSD
KFFFHVPITLNYQAANSPSKFNQRVNAYLKEHPETPIIGIDRGERNLIYIT
VIDSTGKILEQRSLNTIQQFDYQKKLDNREKERVAARQAWSVVGTIKDLKQ
GYLSQVIHEIVDLMIHYQAVVVLENLNFGFKSKRTGIAEKAVYQQFEKMLI
DKLNCLVLKDYPAEKVGGVLNPYQLTDQFTSFAKMGTQSGFLFYVPAPYTS
KIDPLTGFVDPFVWKTIKNHESRKHFLEGFDFLHYDVKTGDFILHFKMNRN
LSFQRGLPGFMPAWDIVFEKNETQFDAKGTPFIAGKRIVPVIENHRFTGRY
RDLYPANELIALLEEKGIVFRDGSNILPKLLENDDSHAIDTMVALIRSVLQ
MRNSNAATGEDYINSPVRDLNGVCFDSRFQNPEWPMDADANGAYHIALKGQ
LLLNHLKESKDLKLQNGISNQDWLAYIQELRN

In one embodiment, the CRISPR-associated nuclease is an engineered Cas9 variant, e.g., a Cas9 Nickase, or a dead Cas9 for use in CRISPRi or CRISPRa systems. For example, variants that nick a single DNA strand instead of creating a double-strand break. (See e.g., Cong, Le, et al. Multiplex genome engineering using CRISPR/Cas systems. Science (2013): 1231143; Mali, Prashant, et al. CAS9 transcriptional activators for target specificity screening and paired nickases for cooperative genome engineering. Nature biotechnology 31.9 (2013): 833; Ran, F. Ann, et al. Double nicking by RNA-guided CRISPR Cas9 for enhanced genome editing specificity. Cell 154.6 (2013): 1380-1389; Cho, Seung Woo, et al. Analysis of off-target effects of CRISPR/Cas-derived RNA-guided endonucleases and nickases. Genome research 24.1 (2014): 132-141, each of which incorporated by reference in their entirety). In some embodiments two guide RNAs are used with the nCAS9. Alternatively, eSpCas9 that uses a single gRNA can be used. Although nickases show high specificity, they rely on two guide RNAs to reach the target sites, thereby reducing the number of potential target sites in the genome. An alternative was created by engineering versions of Cas9 that improved fidelity using a single guide RNA; (see e.g., Qi, Lei S., et al. Repurposing CRISPR as an RNA-guided platform for sequence-specific control of gene expression. Cell 152.5 (2013): 1173-1183, incorporated by reference in its entirety).

In one embodiment, the CRISPR-associated nuclease is SpCas9-HF1 or HypaCas9Kleinstiver (See e.g., Benjamin P., et al. High-fidelity CRISPR-Cas9 nucleases with no detectable genome-wide off-target effects. Nature 529.7587 (2016): 490; Chen, Janice S., et al. Enhanced proofreading governs CRISPR-Cas9 targeting accuracy. Nature 550.7676 (2017): 407, each of which are incorporated by reference in their entirety).

In one embodiment, the CRISPR-associated nuclease is the xCas9 nuclease that recognizes a broad range of PAM sequences, increasing the target sites to 1 in 4 in the genome, (See e.g., Hu, Johnny H., et al. Evolved Cas9 variants with broad PAM compatibility and high DNA specificity. Nature (2018), incorporated by reference in its entirety).

In one embodiment, the CRISPR-associated nuclease is a split Cas9. Fusions with fluorescent proteins like GFP can be made. This would allow imaging of genomic loci (see “Dynamic Imaging of Genomic Loci in Living Human Cells by an Optimized CRISPR/Cas System” Chen B et al. Cell 2013), but in an inducible manner. As such, in some embodiments, one or more of the Cas9 parts may be associated (and in particular fused with) a fluorescent protein, for example GFP. In general, any use that can be made of a Cas9, whether wt, nickase or a dead-Cas9 (with or without associated functional domains) can be pursued using the split Cas9 approach.

In one embodiment, the CRISPR-associated nuclease is a dimeric CRISPR RNA-guided Fokl nuclease (see, e.g., Tsai S G, et al. Nat Biotechnol. 2014. 32(6):569-576, which is incorporated herein by reference in its entirety).

In one embodiment, the CRISPR-associated nuclease is Neisseria meningitidis (NmCas9). NmCas9 is distinct from other known Cas9 nucleases, e.g., from SaCas9 and StCas9, as it recognizes a 5′-NNNNGATT-3′ PAM sequence; see, e.g., Esvelt, K M., et al. Nature Methods (2013); and Hou, Z., et al. PNAS (2013) the contents of which are incorporated herein by reference in their entireties).

In one embodiment, the CRISPR-associated nuclease is a truncated. As used herein, “truncated” refers to a nuclease that has been modified to remove certain amino acids from the wild-type sequence. A truncated nuclease can retain its functionality, e.g., DNA cutting, or it can lack its functionality (e.g., an inactive nuclease). In one embodiment, the CRISPR-associated nuclease is a truncated Cas9. In one embodiment, the CRISPR-associated nuclease is a truncated NmCas9. Sequences of truncated Cas9 nucleases, e.g., NmCas9, are further described in U.S. Patent Application Number 2019/0040371, which is incorporated herein by reference in its entirety.

In one embodiment, the CRISPR-associated nuclease is Inactive Cas9, Dead Cas9 (also referred to as dCAS9). The dead Cas9 (dCas9) CRISPR variant is made by simply inactivating the catalytic nuclease domains while maintaining the recognition domains that allow guide RNA-mediated targeting to specific DNA sequences (Komor, Alexis C., et al. Programmable editing of a target base in genomic DNA without double-stranded DNA cleavage. Nature 533.7603 (2016): 420, incorporated by reference in its entirety). dCas9 is known to silence gene expression by physically blocking the transcription. dCas9 has also been fused to other proteins and used in various applications. For instance, gene activators or inhibitors can be fused to the dCas9 to activate or repress gene expression (CRISPRa and CRISPRi). Also, tagging a fluorescent dye to the dCas9 has enabled visualization of specific DNA fragments the genome (Gaudelli, Nicole M., et al. Programmable base editing of A•T to G•C in genomic DNA without DNA cleavage. Nature 551.7681 (2017): 464, incorporated by reference in its entirety). In one embodiment, FokI fused dCas9 is used (Abudayyeh, Omar O., et al. C2c2 is a single-component programmable RNA-guided RNA-targeting CRISPR effector. Science 353.6299 (2016): aaf557314, incorporated by reference in its entirety).

In one embodiment, the deactivated CRISPR-associated nuclease is a functional gene editing nuclease by serving as a base editor. Base editor enzymes consist of a dead Cas9 domain fused with catalytic enzyme cytidine aminase that converts GC to AT or for example, a tRNA adenosine deaminase fused with Cas9 to convert AT to GC, thus allowing for a complete range of nucleotide exchanges in the genome: See e.g., Komor, Alexis C., et al. Programmable editing of a target base in genomic DNA without double-stranded DNA cleavage. Nature 533.7603 (2016): 420; Gaudelli, Nicole M., et al. Programmable base editing of A•T to G•C in genomic DNA without DNA cleavage. Nature 551.7681 (2017): 464; incorporated by reference in their entirety).

In one embodiment, the Target sequence is RNA and the CRISPR-associated nuclease is an RNA editor such as Cas13a and Cas13b (See e.g., Abudayyeh, Omar 0., et al. RNA targeting with CRISPR-Cas13. Nature 550.7675 (2017): 280; Smargon, Aaron A., et al. Cas13b is a type VI-B CRISPR-associated RNA-guided RNase differentially regulated by accessory proteins Csx27 and Csx28. Molecular cell 65.4 (2017): 618-630; each incorporated by reference in its entirety. In one embodiment the nuclease is Cas13d. The Cas13d family of ribonucleases was identified by scanning sequences of prokaryotes for nucleases resembling previously known Cas13 enzymes. These RNA-guided RNases are about 20% smaller than the Cas13a-Cas13c nucleases, but show comparable targeting efficiency as the previously known variants. The smaller size of these enzymes gives them several advantages, such as being more convenient to package and deliver into cells. (See e.g., Konermann, Silvana, et al. Transcriptome Engineering with RNA-Targeting Type VI-D CRISPR Effectors. Cell (2018); Yan, Winston X., et al. Cas13d Is a Compact RNA-Targeting Type VI CRISPR Effector Positively Modulated by a WYL-Domain-Containing Accessory Protein. Molecular cell (2018), each of which are incorporated by reference in their entirety).

Target polynucleotides, e.g., target sequences, include any polynucleotide sequence to which a co-localization complex as described herein can be useful to either regulate or nick. Target polynucleotides include genes. For purposes of the present disclosure, DNA, such as double stranded DNA, can include the target polynucleotide and a co-localization complex can bind to or otherwise co-localize with the DNA at or adjacent or near the target polynucleotide and in a manner in which the co-localization complex may have a desired effect on the target polynucleotide. Such target polynucleotides can include endogenous (or naturally occurring) polynucleotides and exogenous (or foreign) polynucleotides. One of skill based on the present disclosure will readily be able to identify or design guide RNAs and Cas9 proteins which co-localize to a DNA including a target nucleic acid. One of skill will further be able to identify transcriptional regulator proteins or domains which likewise co-localize to a DNA including a target nucleic acid. DNA includes genomic DNA, mitochondrial DNA, viral DNA or exogenous DNA.

In one embodiment, a target polynucleotide is a disease gene. As used herein, a “disease gene” refers to a gene that has a genetic alteration (e.g., a genetic mutation) that results in, or causes the onset of, a given disease. The genetic alteration can be, but is not limited to, a missense mutation, a nonsense mutation, a substitution, an insertion, a deletion, a duplication, a frameshift mutation, a translocation, an inversion, a repeat expansion, or an encoded cryptic start or stop site. A genetic alteration can result in, for example, increased activity of the gene or gene product, decreased activity of the gene or gene product, alternate splicing of the gene, a truncated gene or gene product, or a lengthened gene or gene product. Said another way, a genetic alteration in a disease gene results in altered activity, function, and/or levels of a gene or gene product as compared to the wild type gene, e.g., the gene not having a genetic mutation. Exemplary diseases and their corresponding disease genes that can be treated with the systems described herein are further described herein below. Disease genes for a given disease are known in the art. One skilled in the art can determine the type of genetic alteration in a given gene in a subject using standard techniques. For example, genome sequencing of a subject with a given disease can be performed, and comparing the genome sequence of a subject that does not have the disease. Using this technique, one skilled in the art can assess the sequence of any gene in the subject's genome, or can focus specifically on a putative or known disease gene.

As used herein, the term “guide RNA” generally refers to an RNA molecule (or a group of RNA molecules collectively) that can bind to a CRISPR-associated nuclease, e.g., an endonuclease, for example, a Cas protein, and aid in targeting the endonuclease to a specific location within a target polynucleotide (e.g., a DNA). A guide RNA can comprise a crRNA segment and a tracrRNA segment. As used herein, the term “crRNA” or “crRNA segment” refers to an RNA molecule or portion thereof that includes a polynucleotide-targeting guide sequence, a stem sequence, and, optionally, a 5′-overhang sequence. As used herein, the term “tracrRNA” or “tracrRNA segment” refers to an RNA molecule or portion thereof that includes a protein-binding segment (e.g., the protein-binding segment is capable of interacting with a CRISPR-associated protein, such as a Cas9). The term “guide RNA” encompasses a single guide RNA (sgRNA), where the crRNA segment and the tracrRNA segment are located in the same RNA molecule. The term “guide RNA” also encompasses, collectively, a group of two or more RNA molecules, where the crRNA segment and the tracrRNA segment are located in separate RNA molecules.

A synthetic guide RNA that has “gRNA functionality” is one that has one or more of the functions of naturally occurring guide RNA, such as associating with an endonuclease, or a function performed by the guide RNA in association with an endonuclease. In certain embodiments, the functionality includes binding a target polynucleotide. In certain embodiments, the functionality includes targeting the endonuclease or a gRNA:endonuclease complex to a target polynucleotide. In certain embodiments, the functionality includes nicking a target polynucleotide. In certain embodiments, the functionality includes cleaving a target polynucleotide. In certain embodiments, the functionality includes associating with or binding to the endonuclease. In certain embodiments, the functionality is any other known function of a guide RNA in a CRISPR-associated nuclease system with an endonuclease, including an artificial CRISPR-associated nuclease system with an engineered endonuclease, for example, an engineered Cas protein. In certain embodiments, the functionality is any other function of natural guide RNA. The synthetic guide RNA may have gRNA functionality to a greater or lesser extent than a naturally occurring guide RNA. In certain embodiments, a synthetic guide RNA may have greater functionality as to one property and lesser functionality as to another property in comparison to a similar naturally occurring guide RNA.

Guide RNAs, e.g., for use with the system described herein are known in the art and are further described in U.S. Pat. No. 9,834,791; and Patent Application No. US2013/0254304. Guide RNAs, e.g., for use with ZFN system are known in the art and are further described in International Patent Application No. W02014/186,585. Patents cited herein are incorporated herein by reference in their entirety.

Guide RNA sequences can be readily generated for a given target sequence using prediction software, for example, CRISPRdirect (available on the world wide web at crispr.dbels.jp/), see Natio, et al. Bioinformatics. (2015) Apr. 1; 31(7): 1120-1123; ATUM gRNA Design Tool (available on the world wide web at atum.bio:ecommerce/cas9/input); an CRISPR-ERA (available on the world wide web at crispr-era.stanford.eduu/indexjsp), see Liu, et al. Bioinformatics, (2015) Nov. 15; 31(22): 3676-3678. All references cited herein are incorporated herein by reference in their entireties. Non-limiting examples of publically available gRNA design software include; sgRNA Scorer 1.0, Quilt Universal guide RNA designer, Cas-OFFinder & Cas-Designer, CRISPR-ERA, CRISPR/Cas9 target online predictor, Off-Spotter—for designing gRNAs, CRISPR MultiTargeter, ZiFiT Targeter, CRISPRdirect, CRISPR design from crispr.mit.edu/, E-CRISP etc.

A guide RNA described herein can be modified, e.g., chemically modified. Exemplary chemical modifications of a guide RNA are described in, for example, Patent Application W02016/089,433, which is incorporated herein by reference in its entirety.

In any of the methods described herein, the oligonucleotide that binds the regulatory sequence and/or small molecule and/or other compound can be introduced into a cell comprising components of the gene editing system described herein and such a cell can be in an animal, which can be a human, non-human mammal (dog, cat, horse, cow, etc.) or other animal.

When a nucleic acid encoding one or more single-guide RNAs and a nucleic acid encoding a CRISPR associated nuclease (RNA-guided nuclease) described herein each need to be administered in vivo, the use of an adenovirus associated vector (AAV) is specifically contemplated. Other vectors for simultaneously delivering nucleic acids to all components of the genome editing/fragmentation system (e.g., sgRNAs, RNA-guided endonuclease) include lentiviral vectors, such as Epstein Barr, Human immunodeficiency virus (HIV), and hepatitis B virus (HBV). Each of the components of the RNA-guided genome editing system (e.g., sgRNA and endonuclease) can be delivered in a separate vector (viral or non-viral) as known in the art or as described herein. In addition, the oligonucleotide component of the gene editing system that binds to the regulatory sequence and prevents splicing resulting in expression of functional nuclease can be delivered by naked DNA, a non-viral vector, or by using a viral vector.

High dosage of a nuclease, for example, Cas9 can exacerbate indel frequencies at off-target sequences which exhibit few mismatches to the guide strand. Such sequences are especially susceptible if mismatches are non-consecutive and/or outside of the seed region of the guide. Herein, we describe a means to mitigate the off-target effects, by specific regulation of nuclease activity, both temporal control and local control of CRISPR associated nuclease activity. The gene editing system described herein, can be used to reduce dosage in long-term expression experiments and therefore result in reduced off-target indels compared to constitutively active CRISPR associated nuclease, e.g., Cas9. In some embodiments, additional methods to minimize the level of toxicity and off-target effect are used and include for example, use of Cas nickase mRNA (for example S. pyogenes Cas9 with the D10A mutation) and a pair of guide RNAs targeting a site of interest, See also WO 2014/093622 (PCT/US2013/074667) herein incorporated by reference in its entirety.

An oligonucleotide that binds the regulatory sequence of this invention is an oligonucleotide (e.g., RNA or DNA or a combination of both) that prevents splicing activity at a specific splice site. The oligonucleotide that binds the regulatory sequence binds to a nucleotide sequence that is a member of the set of splice elements that direct the splicing event, e.g., second set of splice elements, thereby inhibiting splicing. Thus, the oligonucleotide that binds the regulatory sequence can be complementary to a splice junction, a 5′ splice element, a 3′ splice element, a cryptic splice element, a branch point, a cryptic branch point, a native splice element, a mutated splice element, etc. Some nonlimiting examples of an oligonucleotide that binds the regulatory sequence of this invention include GCTATTACCTTAACCCAG (SEQ ID NO:37); specific for the 654T mutation of the globin intron and GCACTTACCTTAACCCAG (SEQ ID NO:38); specific for the 657GT mutation of the globin intron). Other examples include oligonucleotides comprising, consisting essentially of and/or consisting of the nucleotide sequence of SEQ ID NOs:37, 38, 42, 49, 46, 47, 48, 39, 40, 41, 43, 44, 45, 72, 73, 76, 79 and 80. By “consisting essentially of” in the context of these oligonucleotide sequences, it is intended that the oligonucleotide can include additional nucleotides (e.g., 1, 2, 3, 4, 5, 6, 7, 8, 9, or 10 additional) at either the 3′ end or the 5′ end of the oligonucleotide sequence that do not materially affect the function or activity of the oligonucleotide (e.g., these additional nucleotides do not hybridize to the sequence complementary to the original oligonucleotide sequence).

In one embodiment, the oligonucleotide that binds the regulatory domain has a sequence selected from Table 4.

In one embodiment, the oligonucleotide having the sequence of SEQ ID NO: 138 (e.g., LNA-AON1), binds to the regulatory sequence having the sequence of SEQ ID NO: 143.

In one embodiment, the oligonucleotide having the sequence of SEQ ID NO: 139 (e.g., LNA-AON2), binds to the regulatory sequence having the sequence of SEQ ID NO: 144.

In one embodiment, the oligonucleotide having the sequence of SEQ ID NO: 140 (e.g., LNA-AON3), binds to the regulatory sequence having the sequence of SEQ ID NO: 145.

In one embodiment, the oligonucleotide having the sequence of SEQ ID NO: 141 (e.g., LNA-AON4), binds to the regulatory sequence having the sequence of SEQ ID NO: 146.

In one embodiment, the oligonucleotide having the sequence of SEQ ID NO: 142 (e.g., LNA-654), binds to the regulatory sequence having the sequence of SEQ ID NO: 147.

In one embodiment, the regulatory sequence that the oligonucleotide binds is selected from Table 5.

In one embodiment, the regulatory sequence WT 247aa: GGGTTAAG/GCAATAGC has the nucleotide sequence of SEQ ID NO: 148.

(SEQ ID NO: 148)
GTGAGTctat gggacccttg atgttctttt aatatacttt
tttgtttatc ttatttctaa tactttccct aaTCTCTTTC
TTTCAGGgca ataatgatac aatgtatcat gcctctttgc
accattctaa agaataacag tgataatttc tgggttaAGG
CAATAgcaat atttctgcat ataaatattt agtccaagct
aggccctttt gctaatcatg ttcatacctc ttaTCCTCCT
CCCACAG/

In one embodiment, the oligo that binds the WT 247aa regulatory sequence is Oligo

(SEQ ID NO: 149)
5′-GcTaTtGcCtTaAcCc-3′.

In one embodiment, the regulatory sequence IVS2(S0)-654: GGGTTAAG/GTAATAGC has the nucleotide sequence of SEQ ID NO:147.

(SEQ ID NO: 147)
GTGAGTctat gggacccttg atgttctttt aatatacttt
tttgtttatc ttatttctaa tactttccct aaTCTCTTTC
TTTCAGGgca ataatgatac aatgtatcat gcctctttgc
accattctaa agaataacag tgataatttc tgggttaAGG
TAATAgcaat atttctgcat ataaatattt agtccaagct
aggccctttt gctaatcatg ttcatacctc ttaTCCTCCT
CCCACAG/

In one embodiment, the oligo that binds the IVS2(S0)-654 regulatory sequence is

(SEQ ID NO: 142)
Oligo 5′-GcTaTtAcCtTaAcCc-3′.

In one embodiment, the regulatory sequence LUC-AON1: GAGGGCAG/GTGAGTAC has the nucleotide sequence of SEQ ID NO:143.

(SEQ ID NO: 143)
GTGAGTctat gggacccttg atgttctttt aatatacttt
tttgtttatc ttatttctaa tactttccct aaTCTCTTTC
TTTCAGGgca ataatgatac aatgtatcat gcctctttgc
accattctaa agaataacag tgataatttc tgagggcAGG
TGAGTAcaat atttctgcat ataaatattt agtccaagct
aggccctttt gctaatcatg ttcatacctc ttaTCCTCCT
CCCACAG/

In one embodiment, the oligo that binds the LUC-AON1 regulatory sequence is

(SEQ ID NO: 138)
Oligo 5′-GtAcTcAcCtGcCcTc-3′.

In one embodiment, the regulatory sequence LUC-AON2: GTGCCGAG/GTAAGTTC has the nucleotide sequence of SEQ ID NO: 144.

(SEQ ID NO: 144)
GTGAGTctat gggacccttg atgttctttt aatatacttt
tttgtttatc ttatttctaa tactttccct aaTCTCTTTC
TTTCAGGgca ataatgatac aatgtatcat gcctctttgc
accattctaa agaataacag tgataatttc tgTgccgAGG
TAAGTTcaat atttctgcat ataaatattt agtccaagct
aggccctttt gctaatcatg ttcatacctc ttaTCCTCCT
CCCACAG/

In one embodiment, the oligo that binds the LUC-AON2 regulatory sequence is

(SEQ ID NO: 139)
Oligo 5′-GaAcTtAcCtCgGcAc-3′.

In one embodiment, the regulatory sequence LUC-AON3: CTGACTAG/GTGAGTCC has the nucleotide sequence of SEQ ID NO: SEQ ID NO: 145.

(SEQ ID NO: 145)
GTGAGTctat gggacccttg atgttctttt aatatacttt
tttgtttatc ttatttctaa tactttccct aaTCTCTTTC
TTTCAGGgca ataatgatac aatgtatcat gcctctttgc
accattctaa agaataacag tgataatttc tcTgactAGG
TGAGTCcaat atttctgcat ataaatattt agtccaagct
aggccctttt gctaatcatg ttcatacctc ttaTCCTCCT
CCCACAG/

In one embodiment, the oligo that binds the LUC-AON3 regulatory sequence is

(SEQ ID NO: 140)
Oligo 5′-GgAcTcAcCtAgTcAg-3′.

In one embodiment, the regulatory sequence Luc-AON4: GCCAATAG/GTAAGTGC has the nucleotide sequence of SEQ ID NO: 146.

(SEQ ID NO: 146)
GTGAGTctat gggacccttg atgttctttt aatatacttt
tttgtttatc ttatttctaa tactttccct aaTCTCTTTC
TTTCAGGgca ataatgatac aatgtatcat gcctctttgc
accattctaa agaataacag tgataatttc tgccaatAGG
TAAGTGcaat atttctgcat ataaatattt agtccaagct
aggccctttt gctaatcatg ttcatacctc ttaTCCTCCT
CCCACAG/

In one embodiment, the oligo that binds the LUC-AON4 regulatory sequence is

(SEQ ID NO: 141)
Oligo 5′-GcAcTtAcCtAtTgGc-3′.

The oligonucleotide that binds the regulatory sequence can, in some embodiments, be an oligonucleotide that does not activate RNase H. Oligonucleotides that do not activate RNase H can be made in accordance with known techniques. See, e.g., U.S. Pat. No. 5,149,797 to Pederson et al. Such oligonucleotides, which can be deoxyribonucleotide or ribonucleotide sequences, contain any structural modification which sterically hinders or prevents binding of RNase H to a duplex molecule containing the oligonucleotide as one member thereof, which structural modification does not substantially hinder or disrupt duplex formation. Because the portions of the oligonucleotide involved in duplex formation are substantially different from those portions involved in RNase H binding thereto, numerous oligonucleotides that do not activate RNase H are available.

Oligonucleotides of this invention can also be oligonucleotides wherein at least one, or all, of the internucleotide bridging phosphate residues are modified phosphates, such as methyl phosphonates, methyl phosphorothioates, phosphoromorpholidates, phosphoropiperazidates and phosphoramidates. As an additional example, every other one of the internucleotide bridging phosphate residues can be modified as described. In another non-limiting example, such oligonucleotides are oligonucleotides wherein at least one, or all, of the nucleotides contain a 2′ lower alkyl moiety (e.g., C1-C4, linear or branched, saturated or unsaturated alkyl, such as methyl, ethyl, ethenyl, propyl, 1-propenyl, 2-propenyl, and isopropyl). For example, every other one of the nucleotides can be modified as described. (See also Furdon et al., Nucleic Acids Res. 17:9193-9204 (1989); Agrawal et al., Proc. Natl. Acad. Sci. USA 87:1401-1405 (1990); Baker et al., Nucleic Acids Res. 18, 3537-3543 (1990); Sproat et al., Nucleic Acids Res. 17:3373-3386 (1989); Walder and Walder, Proc. Natl. Acad. Sci. USA 85:5011-5015 (1988).) Thus, in some embodiments, the blocking nucleotide of this invention can comprise a modified internucleotide bridging phosphate residue that can be, but is not limited to, a methyl phosphorothioate, a phosphoromorpholidate, a phosphoropiperazidate and/or a phosphoramidate, in any combination. In certain embodiments, the blocking can comprise a nucleotide having a lower alkyl substituent at the 2′ position thereof.

An oligonucleotide that binds the regulatory sequence described herein can be modified, for example, by a small molecule, to increase its recruitment to RNA in the cell. An oligonucleotide modified in this manner will have increased efficiency for binding and cleaving the RNA when co-expressed in a cell with the small molecule. Further review of this modification can be found, e.g., in Costales, M G, et al. J. Am. Chem. Soc. 2081, 140; 6741-6744; U.S. Patent Application No. US2008/0227213A1; and International Patent No. WO 2015/021415A1; each of which are incorporated herein by reference in their entireties.

An oligonucleotide that binds the regulatory sequence herein can be modified, for example, to increase the oligonucleotide's permeability, affinity, stability (e.g., to prevent its degradation), and pharmacodynamics properties. Examples of such modifications include, but are not limited to, peptide nucleic acids (PNA) and locked nucleic acids (LNA). Further review of these modification can be found, e.g., in Havens, M A, et al. Nucleic Acids Research. 2016: 44(14); 6549-6563, which is incorporated herein by reference in its entirety.

In a PNA, the backbone is made from repeating N-(2-aminoethyl)-glycine to units linked by peptide bonds. The different bases (purines and pyrimidines) are linked to the backbone by methylene carbonyl linkages. Unlike DNA or other DNA analogs, PNAs do not contain any pentose sugar moieties or phosphate groups. PNAs are depicted like peptides with the N-terminus at the first (left) position and the C-terminus at the right. The PNA backbone is not charged and this confers to this polymer a much stronger binding between PNA/DNA strands than between PNA strands and DNA strands. This is due to the lack of charge repulsion between PNA and DNA strands.

Early experiments with homopyrimidine strands have shown that the Tni of a 6-mer PNA T/DNA dA was determined to be 31° C. in comparison to a DNA dT/DNA dA 6-mer duplex that denatures at a temperature less than 10° C.

PNAs with their peptide backbone bearing purine and pyrimidine bases are not a molecular species easily recognized by nucleases or proteases. They are thus resistant to enzyme degradation. PNAs are also stable over a wide pH range. Because they are not easily degraded by enzymes, the lifetime of these polymers is extended both in vitro and in vivo. In addition, the fact that they are not charged facilitates their crossing through cell membranes and their stronger binding properties should decrease the amount of oligonucleotide needed for the regulation of gene expression.

LNAs are a class of nucleic acids containing nucleosides whose major distinguishing characteristic is the presence of a methylene bridge between the 2′-0 and 4′-C atoms of the ribose ring. This bridge restricts the flexibility of the ribofuranose ring of the nucleotide analog and locks it into the rigid bicyclic N-type conformation. Furthermore, LNA induces adjacent DNA bases to adopt this conformation, resulting in the formation of the more thermodynamically stable form of the A duplex LNA nucleosides containing the four common nucleobases that appear in DNA (A,T,G,C) that can base-pair with their complementary nucleosides according to standard Watson-Crick rules. LNA can be mixed with DNA or RNA, as well as other nucleic acid analogs using standard phosphoramidite DNA synthesis chemistry. Therefore, LNA oligonucleotides can easily be tagged with, e.g., amino-linkers, biotin, fluorophores, etc. Thus, a very high degree of freedom in the design of primers and probes exists. Their locked conformation increases binding affinity for complementary sequences and provides a new chemical approach to optimize and fine tune primers and probes for sensitive and specific detection of nucleic acids. This difference is observable experimentally as an increased thermal stability of LNA-NA heteroduplexes and is dependent both on the number of LNA nucleosides present in the sequence, as well as the chemical nature of the nucleobases employed. This experimental difference can be exploited to modulate the specificity of oligonucleotide probes designed to detect specific nucleic acids targets through standard hybridization techniques.

As used herein, “a member of the second set of splice elements” includes any element that is involved in activation of splicing of the second intron from the pre-mRNA. For example, element of the second set of splice elements can be the result of a mutation in the native DNA and/or pre-mRNA that can be either a substitution mutation and/an addition mutation and/or a deletion mutation that creates a new splice element. The new splice element is thus one member of a second set of splice elements that define a second intron. The remaining members of the second set of splice elements can also be members of the set of splice elements that define the first intron. For example, if the mutation creates a new, second 3′ splice site which is both upstream from (i.e., 5′ to) the first 3′ splice site and downstream from (i.e., 3′ to) a first branch point, then the first 5′ splice site and the first branch point can serve as members of both the first set of splice elements and the second set of splice elements.

In some situations, the introduction of a second set of splice elements can cause native regions of the RNA that are normally dormant, or play no role as splicing elements, to become activated and serve as splicing elements. Such elements are referred to as “cryptic” elements. For example, if a new 3′ splice site is introduced, which is situated between the first 3′ splice site and the first branch point, it can activate a cryptic branch point between the new 3′ splice site and the first branch point.

In other situations, the introduction of a new 5′ splice site that is situated between the first branch point and the first 5′ splice site can further activate a cryptic 3′ splice site and a cryptic branch point sequentially upstream from the new 5′ splice site. In this situation, the first intron becomes divided into two aberrant introns, with a new exon situated therebetween.

Further, in some situations where a first splice element (particularly a branch point) is also a member of the set of second splice elements, it can be possible to block the first element and activate a cryptic element (i.e., a cryptic branch point) that will recruit the remaining members of the first set of splice elements to force correct splicing over incorrect splicing. Note further that, when a cryptic splice element is activated, it can be situated in either the intron and/or in one of the adjacent exons. Thus as indicated above, depending on the set of splice elements that make up the “second set of splice elements,” the oligonucleotide that binds the regulatory sequence, small molecule and/or other compound of this invention can block a variety of different splice elements to carry out the instant invention. For example, it can block a mutated element, a cryptic element, a native element, a 5′ splice site, a 3′ splice site, and/or a branch point. In general, it will not block a splice element which also defines the first intron, of course taking into account the situation where blocking a splice element of the first intron activates a cryptic element which then serves as a surrogate member of the first set of splice elements and participates in correct splicing, as discussed above.

The length of the oligonucleotide that binds the regulatory sequence (i.e., the number of nucleotides therein) is not critical so long as it binds selectively to the intended location, and can be determined in accordance with routine procedures. Thus, in some embodiments, the oligonucleotide that binds the regulatory sequence of this invention can be between about 5 and about 100 nucleotides in length. In particular, a blocking nucleotide of this invention can be about 5, 6, 7, 8, 9, 10, 11, 12, 13, 14, 15, 16, 17, 18, 19, 20, 21, 22, 23, 24, 25, 26, 27, 30 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or 100 nucleotides in length. In some embodiments, the oligonucleotide that binds the regulatory sequence of this invention is from eight to 50 nucleotides in length. In yet other embodiments of this invention, the oligonucleotide that binds the regulatory sequence is 15-25 nucleotides in length and can also be 18-20 nucleotides in length. An oligonucleotide that binds the regulatory sequence can be used in a method described herein as a population of identical oligonucleotides and/or as a population of different oligonucleotides present in any combination and/or in any ratio relative to one another.

A small molecule of this invention is an active chemical compound that can be structurally and/or functionally diverse in comparison with other small molecules and that has a low molecular weight (e.g., less than 5,000 Daltons). A small molecule can be a natural or synthetic substance. They can be synthesized by organic chemistry protocols and/or isolated from natural sources, such as plants, fungi and microbes. A small molecule can be “drug-like” (e.g., aspirin, penicillin, chemotherapeutics), toxic and/or natural. A small molecule drug can be one or more active chemical compounds, typically formulated as an orally available pill, that interact with a specific biological target, such as a receptor, enzyme or ion channel, to provide a therapeutic effect. Specific but nonlimiting examples of a small molecule of this invention include antibiotics, nucleoside analogs (e.g., toyocamycin) and aptamers (e.g., RNA aptamers; DNA aptamers).

A small molecule of this invention can be a small molecule present in any number of small molecule libraries, some of which are available commercially. Nonlimiting examples of libraries that can contain a small molecule of this invention include small molecule libraries obtained from various commercial entities, for example, SPECS and BioSPEC B.V. (Rijswijk, the Netherlands), Chembridge Corporation (San Diego, Calif.), Comgenex USA Inc., (Princeton, N.J.), Maybridge Chemical Ltd. (Cornwall, UK), and Asinex (Moscow, Russia). One representative example is known as DIVERSet™, available from ChemBridge Corporation, 16981 Via Tazon, Suite G, San Diego, Calif. 92127. DIVERSet™ contains between 10,000 and 50,000 drug-like, hand-synthesized small molecules. The compounds are pre-selected to form a “universal” library that covers the maximum pharmacophore diversity with the minimum number of compounds and is suitable for either high throughput or lower throughput screening. For descriptions of additional libraries, see, for example, Tan et al. “Stereoselective Synthesis of Over Two Million Compounds Having Structural Features Both Reminiscent of Natural Products and Compatible with Miniaturized Cell-Based Assays” Am. Chem Soc. 120, 8565-8566, 1998; Floyd et al. Prog Med Chem 36:91-168, 1999. Numerous libraries are commercially available, e.g., from AnalytiCon USA Inc., P.O. Box 5926, Kingwood, Tex. 77325; 3-Dimensional Pharmaceuticals, Inc., 665 Stockton Drive, Suite 104, Exton, Pa. 19341-1151; Tripos, Inc., 1699 Hanley Rd., St. Louis, Mo., 63144-2913, etc.

The small molecules and other compounds of this invention can operate by a variety of mechanisms to modify a splicing event in the nucleic acid of this invention. For example, the small molecules and other compounds of this invention can interfere with the formation and/or function and/or other properties of splicing complexes, spliceosomes, and their components such as hnRNPs, snRNPs, SR-proteins and other splicing factors or elements, resulting in the prevention and/or induction of a splicing event in a pre-mRNA molecule. As another example, the small molecules and other compounds of this invention can prevent and/or modify transcription of gene products, which can include, for example, but are not limited to, hnRNPs, snRNPs, SR-proteins and other splicing factors, which are subsequently involved in the formation and/or function of a particular spliceosome. The small molecules and other compounds of this invention can also prevent and/or modify phosphorylation, glycosylation and/or other modifications of gene products, including but not limited to, hnRNPs, snRNPs, SR-proteins and other splicing factors, which are subsequently involved in the formation and/or function of a particular spliceosome. Additionally, the small molecules and other compounds of this invention can bind to and/or otherwise affect specific pre-mRNA so that a specific splicing event is prevented or induced via a mechanism that does not involve basepairing with RNA in a sequence-specific manner.

The present invention further provides a method of gene editing in a subject, comprising: a) introducing into the subject the gene editing system of this invention; and b) introducing into the subject an oligonucleotide that binds the regulatory sequence and/or small molecule and/or other compound of this invention that blocks a member of the second set of splice elements, thereby producing the protein and/or RNA that imparts a biological function in the subject.

The degree of gene editing that occurs in a subject can be monitored over time according to art-known methods and when the amount falls below a desired and/or therapeutic level, the oligonucleotide that binds the regulatory sequence, small molecule and/or other compound can be introduced into the subject to increase production of the protein and/or RNA, thus regulating the production.

In the methods described herein wherein the gene editing system of this invention is administered to a subject, the nucleic acid, vector and/or cell can initially be present in the subject in the absence of, or the absence of the expression of, an oligonucleotide that binds the regulatory sequence and/or small molecule and/or other compound, the presence of which would result in blocking of a member of the second set of splice elements. In this status, the second set of splice elements is active and there is no or very minimal (e.g., insignificant) production in the subject of the exogenous protein, peptide and/or RNA that imparts a biological function, as encoded by the nuclease sequence. When the oligonucleotide that binds the regulatory sequence, small molecule and/or other compound of this invention is present in the subject, a member of the second set of splice elements on the nucleic acid is blocked, resulting in removal of the first intron by splicing and subsequent production, in the subject, of the protein and/or RNA encoded by the nuclease sequence that imparts a biological function, e.g., gene editing.

The oligonucleotide that binds the regulatory sequence, small molecule and/or other compound can be introduced into the subject at any time relative to the introduction into the subject of the gene editing system of this invention. For example, the oligonucleotide that binds the regulatory sequence, small molecule and/or other compound can be introduced into the subject before, simultaneously with and/or after introduction of the nucleic acid, vector and/or cell into the subject. Furthermore, the oligonucleotide that binds the regulatory sequence, small molecule and/or other compound can be administered one time or at multiple times over any time interval and can extend to throughout the lifespan of the subject.

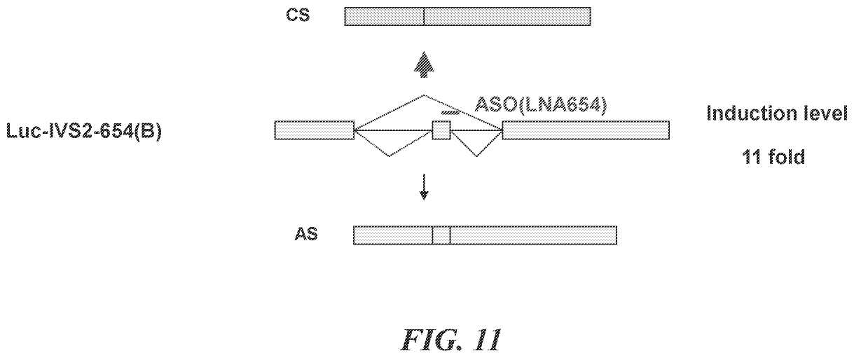

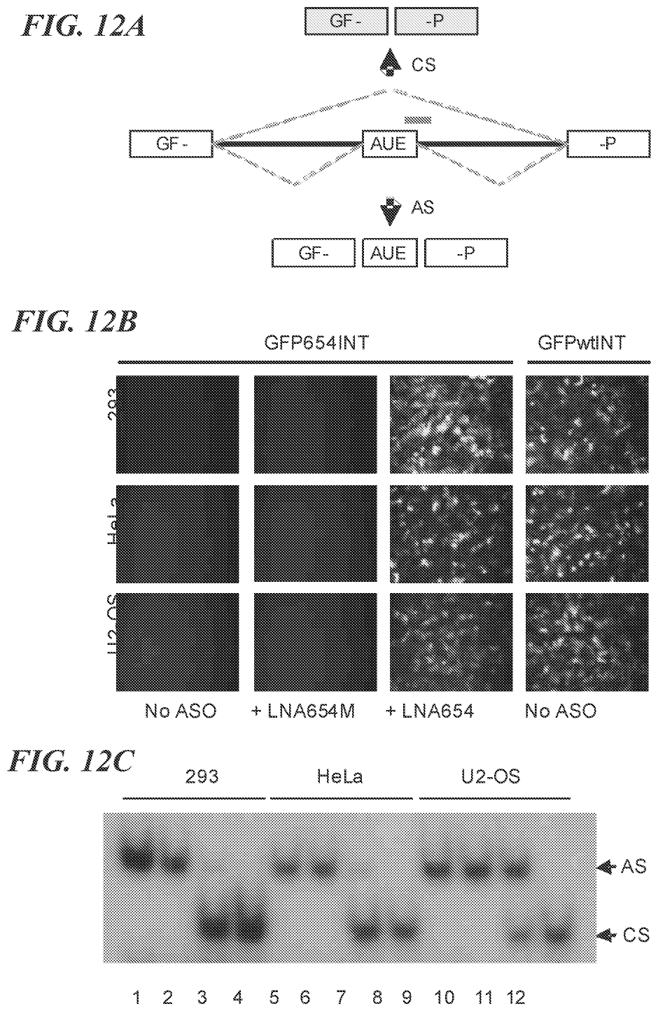

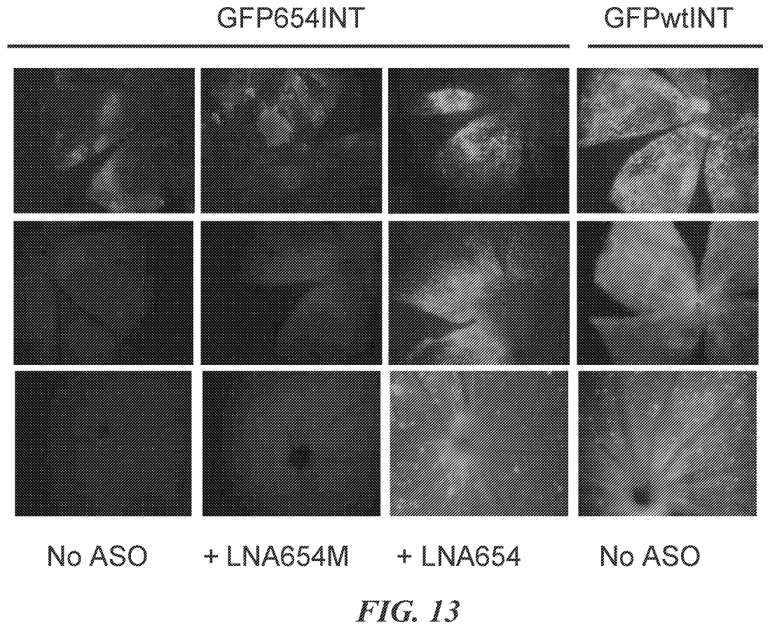

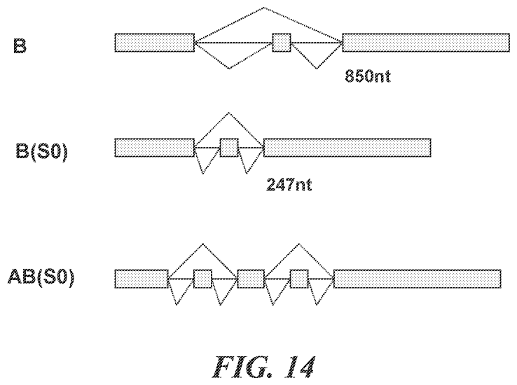

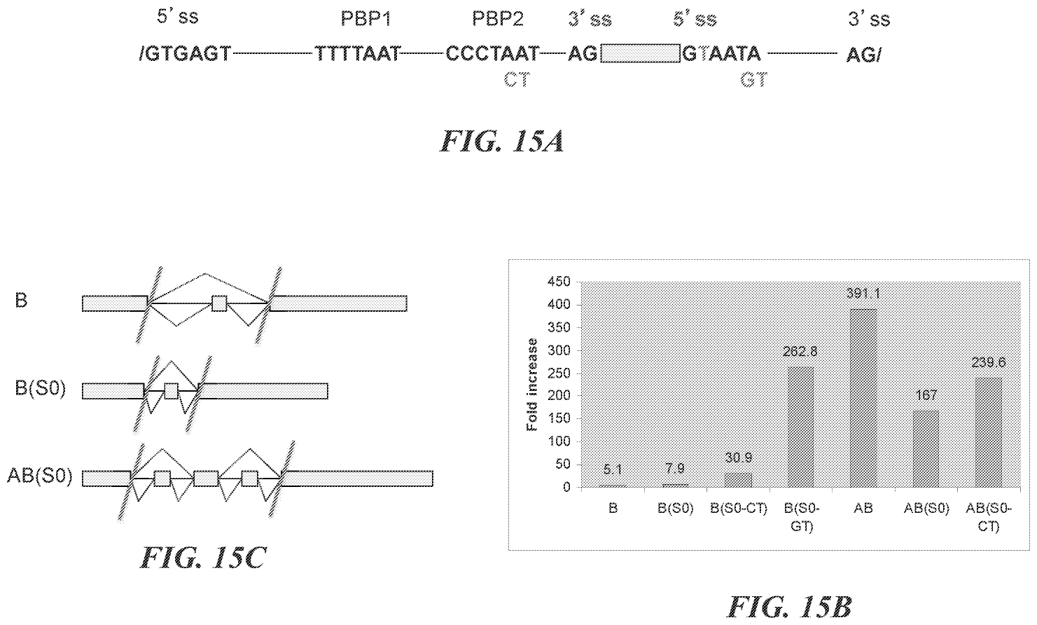

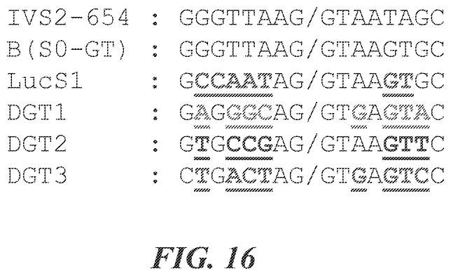

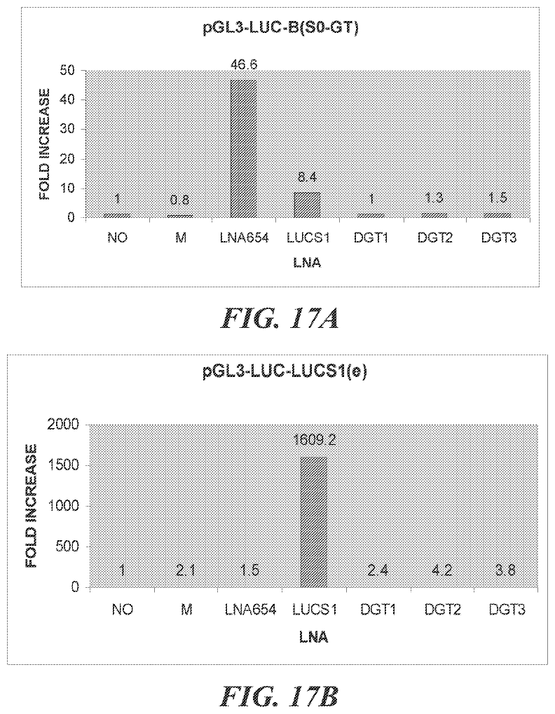

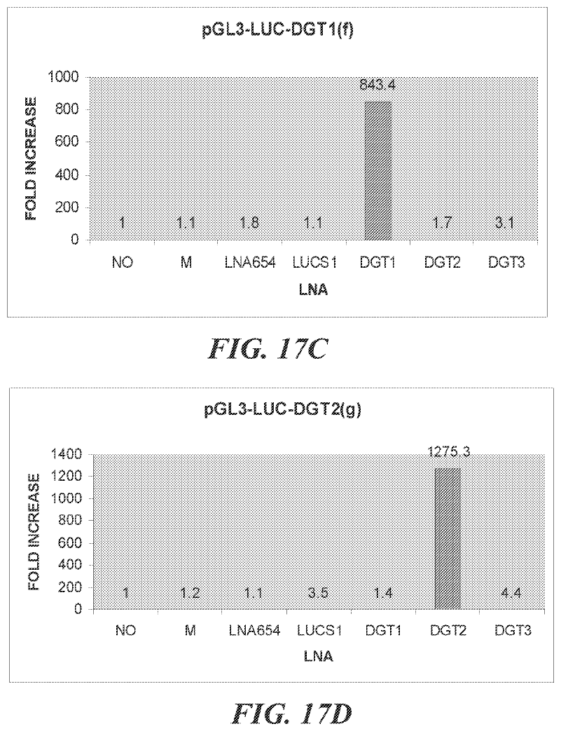

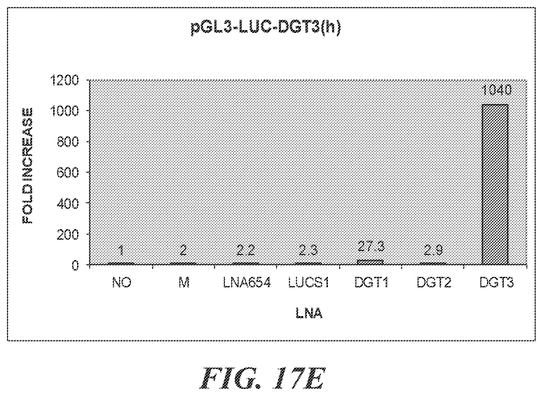

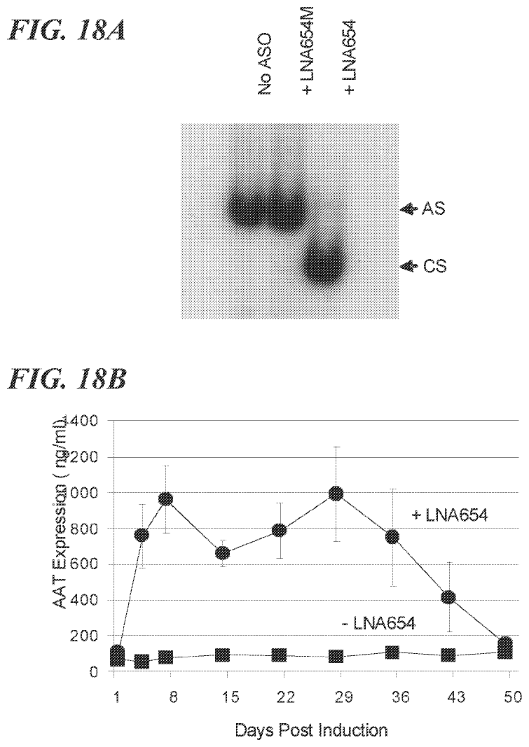

Thus, in some embodiments, the present invention provides a method of treating a disease or disorder in a subject, comprising: a) introducing into the subject an effective amount of the gene editing system of this invention; and b) introducing into the subject an effective amount of an oligonucleotide that binds the regulatory sequence, small molecule, and/or other compound of this invention, thereby treating the disorder in the subject. When the nucleic acid, vector and/or cell and the oligonucleotide that binds the regulatory sequence, small molecule and/or other compound are present in the subject, they are present under conditions whereby the oligonucleotide that binds the regulatory sequence, small molecule and/or other compound can contact the nucleic acid and block a member of the second set of splice elements, thereby resulting in the production of a protein, peptide and/or RNA that imparts a biological function in the subject. See for example FIG. 11; when the second set of splice elements is blocked by an oligo binding to the regulatory sequence (ASO(LNA544)), an mRNA that encodes the correct protein without a non-naturally occurring exon is produced (CS). However, when the oligonucleotide is absent, the first and second intron are individually spliced from the pre-mRNA resulting in a mRNA comprising the non-naturally occurring exon (e.g., that comprises an in-frame stop codon), and non-functional protein is produced (AS).

In additional embodiments, regulation of gene expression according to the methods of this invention can occur in the reverse of the system described herein. Specifically, in some embodiments, the system is in the “OFF” position as described herein in the presence of an oligonucleotide that binds the regulatory sequence, small molecule and/or other compound that regulates splice-mediated expression (e.g., no functional protein is produced).

In one embodiment, the “ON” and “OFF” control of the gene editing system described herein is selectively controlled, for example, under spatial control. For example, the components of the system can be delivered/administered locally to a desired site, location, organ, cell type, tissue type, etc., to induce the gene editing system to turn “ON” locally. It is not required that all components be delivered/administered locally. In one embodiment, components (a) and (b) can be administered systemically, and component (c) can be administered locally, resulting in local control (e.g., turning “ON”) of the gene editing system. In one embodiment, components (a) and (b) can be administered locally, and component (c) is administered systemically. Local delivery of a component of the gene editing system can be achieved by direct delivery of the component to a specific location. Alternatively, local delivery can be achieved using a localization sequence that drives the component to a specific location, or specific promoters that allow for expression of the component in a specific location. In one embodiment, local delivery is achieved by direct injection, e.g., to muscle, heart, or other organ.

In another embodiment, the “ON” and “OFF” control of the gene editing system described herein is selectively controlled, for example, under temporal control. For example, the components of the gene editing system can be administered for a given duration to control the timing in which the system is “ON” or “OFF”. For example, pulsed administration (e.g., discontinuous administration) of component (c) could result in the gene editing system repeatedly turning “ON” and “OFF”.

In one embodiment, the “ON” and “OFF” control of the gene editing system described herein is selectively controlled under both spatial and temporal control.

### Treatment

An “effective amount” of a gene editing system, an oligonucleotide that binds the regulatory sequence, small molecule and/or other compound of this invention refers to a nontoxic but sufficient amount to provide a desired effect, which can be a beneficial and/or therapeutic effect. As is well understood in the art, the exact amount required will vary from subject to subject, depending on age, gender, species, general condition of the subject, the severity of the condition being treated, the particular agent administered, and the like. An appropriate “effective” amount in any individual case may be determined by one of skill in the art by reference to the pertinent texts and literature (e.g., Remington's Pharmaceutical Sciences (latest edition) and/or by using routine pharmacological procedures.

“Treat” or “treating” as used herein refers to any type of treatment that imparts a benefit to a subject that is diagnosed with, at risk of having, suspected to have and/or likely to have a disease or disorder that can be responsive in a positive way to a protein and/or RNA of this invention. A benefit can include an improvement in the condition of the subject (e.g., in one or more symptoms), delay and/or reversal in the progression of the condition, prevention or delay of the onset of the disease or disorder, etc.

Nonlimiting examples of diseases and/or disorders that can be treated by methods of this invention and some examples of the gene product that can be encoded by the nuclease sequence of this invention and that can impart a therapeutic effect include metabolic diseases such as diabetes (insulin), growth/development disorders (growth hormone; zinc finger proteins that regulate growth factors), blood clotting disorders (e.g., hemophilia A (Factor VIII); hemophilia B (Factor IX)), central nervous system disorders (e.g., seizures, Parkinson's disease (glial derived neurotrophic factor (GDNF) and GDNF-like growth factors), Alzheimer's disease (nerve growth factor, GDNF and GDNF-like growth factors), amyotrophic lateral sclerosis, demyelination disease), bone allograft (bone morphogenic protein 2 (proteins 1-9, e.g., MBP2)), inflammatory disorders (e.g., arthritis, autoimmune disease), obesity, cancer, cardiovascular disease (e.g., congestive heart failure (phospholamban and genes related to Ca pump)), macular degeneration (pigment epithelium derived factor (PDEF), 13-thalassemia, a-thalassemia, Tay-Sachs syndrome, phenylketonuria, cystic fibrosis and/or viral infection.

Additional examples include nucleic acids encoding soluble CD4, used in the treatment of AIDS and α-antitrypsin, used in the treatment of emphysema caused by α-antitrypsin deficiency. Other diseases, syndromes and conditions that can be treated by the methods and compositions of this invention include, for example, adenosine deaminase deficiency, sickle cell deficiency, brain disorders such as Huntington's disease, lysosomal storage diseases, Gaucher's disease, Hurler's disease, Krabbe's disease, motor neuron diseases such as dominant spinal cerebellar ataxias (examples include SCA1, SCA2, and SCA3), thalassemia, hemophilia, phenylketonuria, and heart diseases, such as those caused by alterations in cholesterol metabolism, and defects of the immune system. Other diseases that can be treated by these methods include metabolic disorders such as musculoskeletal diseases, cardiovascular disease and cancer. The gene editing system of this invention can also be delivered to airway epithelia to treat genetic diseases such as cystic fibrosis, pseudohypoaldosteronism, and immotile cilia syndrome, as well as non-genetic disorders (e.g., bronchitis, asthma). The gene editing system of this invention can also be delivered to alveolar epithelia to treat genetic diseases like α-l-antitrypsin, as well as pulmonary disorders (e.g., treatment of pneumonia and emphysema pulmonary fibrosis, pulmonary edema; delivery of nucleic acid encoding surfactant protein to premature babies or patients with ARDS).

In general, the gene editing system of the present invention can be employed to deliver any nucleic acid with a biological function to treat or ameliorate the symptoms associated with any disorder related to gene expression. Illustrative disease states include, but are not limited to: cystic fibrosis (and other diseases of the lung), hemophilia A, hemophilia B, thalassemia, anemia and other blood disorders, AIDS, cancer (e.g., brain tumors), diabetes mellitus, muscular dystrophies (e.g., Duchenne, Becker), Gaucher's disease, Hurler's disease, adenosine deaminase deficiency, glycogen storage diseases and other metabolic defects, mucopolysaccharide disease, and diseases of solid organs (e.g., brain, liver, kidney, heart, lung, eye), and the like.

In certain embodiments, the delivery vectors of the invention may be administered to treat diseases of the CNS, including genetic disorders, neurodegenerative disorders, psychiatric disorders and/or tumors. Illustrative diseases of the CNS include, but are not limited to, Alzheimer's disease, Parkinson's disease, Huntington's disease, Rett Syndrome, Canavan disease, Leigh's disease, Refsum disease, Tourette syndrome, primary lateral sclerosis, amyotrophic lateral sclerosis, progressive muscular atrophy, Pick's disease, muscular dystrophy, multiple sclerosis, myasthenia gravis, Binswanger's disease, trauma due to spinal cord or head injury, Tay Sachs disease, Lesch-Nyan disease, epilepsy, cerebral infarcts, psychiatric disorders including mood disorders (e.g., depression, bipolar affective disorder, persistent affective disorder, secondary mood disorder), schizophrenia, drug dependency (e.g., alcoholism and other substance dependencies), neuroses (e.g., anxiety, obsessional disorder, somatoform disorder, dissociative disorder, grief, post-partum depression), psychosis (e.g., hallucinations and delusions), dementia, paranoia, attention deficit disorder, psychosexual disorders, sleeping disorders, pain disorders, eating or weight disorders (e.g., obesity, cachexia, anorexia nervosa, and bulimia) and cancers and tumors (e.g., pituitary tumors) of the CNS.

Disorders of the CNS that can be treated according to the methods of this invention include ophthalmic disorders involving the retina, posterior tract, and optic nerve (e.g., retinitis pigmentosa, diabetic retinopathy and other retinal degenerative diseases, uveitis, age-related macular degeneration, glaucoma).

Most, if not all, ophthalmic diseases and disorders are associated with one or more of three types of indications: (1) angiogenesis, (2) inflammation, and (3) degeneration. The delivery vectors of the present invention can be employed to deliver anti-angiogenic factors; anti-inflammatory factors; factors that retard cell degeneration, promote cell sparing, or promote cell growth and combinations of the foregoing.

Diabetic retinopathy, for example, is characterized by angiogenesis. Diabetic retinopathy can be treated by delivering one or more anti-angiogenic factors either intraocularly (e.g., in the vitreous) or periocularly (e.g., in the sub-Tenon's region). One or more neurotrophic factors can also be co-delivered, either intraocularly (e.g., intravitreally) or periocularly. Uveitis involves inflammation. One or more anti-inflammatory factors can be administered by intraocular (e.g., vitreous or anterior chamber) administration of a nucleic acid of the invention.

Retinitis pigmentosa, by comparison, is characterized by retinal degeneration. In representative embodiments, retinitis pigmentosa can be treated by intraocular (e.g., vitreal) administration of a delivery vector encoding one or more neurotrophic factors. Age-related macular degeneration involves both angiogenesis and retinal degeneration. This disorder can be treated by administering the gene editing system of this invention encoding one or more neurotrophic factors intraocularly (e.g., vitreous) and/or one or more anti-angiogenic factors intraocularly or periocularly (e.g., in the sub-Tenon's region).

Glaucoma is characterized by increased ocular pressure and loss of retinal ganglion cells. Treatments for glaucoma include administration of one or more neuroprotective agents that protect cells from excitotoxic damage using the inventive delivery vectors. Such agents include N-methyl-D-aspartate (NMDA) antagonists, cytokines, and neurotrophic factors, delivered intraocularly, preferably intravitreally.

In other embodiments, the present invention can be used to treat seizures, e.g., to reduce the onset, incidence and/or severity of seizures. The efficacy of a therapeutic treatment for seizures can be assessed by behavioral (e.g., shaking, ticks of the eye or mouth) and/or electrographic means (most seizures have signature electrographic abnormalities). Thus, the invention can also be used to treat epilepsy, which is marked by multiple seizures over time.

As a further example, somatostatin (or an active fragment thereof) can be administered to the brain using a delivery vector of the invention to treat a pituitary tumor. According to this embodiment, the delivery vector encoding somatostatin (or an active fragment thereof) can be administered by microinfusion into the pituitary. Likewise, such treatment can be used to treat acromegaly (abnormal growth hormone secretion from the pituitary). The nucleic acid (e.g., GenBank Accession No. J00306) and amino acid (e.g., GenBank Accession No. P01166; contains processed active peptides somatostatin-28 and somatostatin-14) sequences of somatostatins are known in the art.

In other embodiments, an alternate splicing event can be modulated by employing the gene editing system of this invention. For example, the gene editing system of this invention can be introduced into a subject along with an oligonucleotide that binds the regulatory sequence, small molecule and/or other compound of this invention to produce a first protein and/or RNA that imparts a biological function in the subject as a result of activation at a particular set of splice sets. The same nucleic acid can be engineered to encode a different protein, peptide and/or RNA that imparts a biological function in the subject by activating a different set of splice sets. The different protein and/or RNA is produced when a different oligonucleotide that binds the regulatory sequence, small molecule and/or compound of this invention is introduced into the subject. As an example, the first RNA could produce a first protein of interest when a first oligonucleotide that binds the regulatory sequence, small molecule and/or other compound is present and after addition of a different, second oligonucleotide that binds the regulatory sequence, small molecule and/or compound of this invention, a second RNA can result, that produces a second protein or functional RNA of interest (e.g., an isoform of the first protein could be produced (e.g., interleukin (IL)-4 and its splice variant, IL-4A2). (See, e.g., Fletcher et al. “Increased expression of mRNA encoding interleukin (IL)-4 and its splice variant IL-4A2 in cells from contacts of Mycobacterium tuberculosis, in the absence of in vitro stimulation” Immunology 2004 August; 112(4):669-73; Minn et al. “Insulinomas and expression of an insulin splice variant” Lancet 2004 Jan. 31; 363(9406):363-7; Schlueter et al. “Tissue-specific expression patterns of the RAGE receptor and its soluble forms—a result of regulated alternative splicing?” Biochim Biophys Acta 2003 Oct. 20; 1630(1):1-6; Vegran et al. “Implication of alternative splice transcripts of caspase-3 and survivin in chemoresistance” Bull Cancer 2005 March; 92(3):219-26; Ren et al. “Alternative splicing of vitamin D-24-hydroxylase: A novel mechanism for the regulation of extra-renal 1,25-dihydroxyvitamin D synthesis” J Biol Chem. 2005 Mar. 23; et al. “Mutant huntington protein: a substrate for transglutaminase 1, 2, and 3” J Neuropathol Exp Neurol 2005 January; 64(1):58-65; Ding and Keller. “Splice variants of the receptor for advanced glycosylation end products (RAGE) in human brain” Neurosci Lett. 2005 Jan. 3; 373(1):67-72; et al. “Transcript scanning reveals novel and extensive splice variations in human 1-type voltage-gated calcium channel, Cav1.2 al subunit” J Biol Chem 2004 Oct. 22; 279(43):44335-43, Epub 2004 Aug. 6. All of these references are incorporated by reference herein in their entireties.)

The present invention further provides the gene editing system of this invention in compositions. Thus, in additional embodiments, the present invention provides a composition comprising the gene editing system of this invention, the vector of this invention and/or the cell of this invention, in a pharmaceutically acceptable carrier. By “pharmaceutically acceptable carrier” is meant a carrier that is compatible with other ingredients in the pharmaceutical composition and that is not harmful or deleterious to the subject. In particular, it is intended that a pharmaceutically acceptable carrier be a sterile carrier that is formulated for administration to or delivery into a subject of this invention.

Pharmaceutical compositions comprising a composition of this invention and a pharmaceutically acceptable carrier are also provided. The compositions described herein can be formulated for administration in a pharmaceutical carrier in accordance with known techniques. See, e.g., Remington, The Science And Practice of Pharmacy (latest edition). The carrier may be a solid or a liquid, or both, and is preferably formulated with the composition of this invention as a unit-dose formulation, for example, a tablet, which may contain from about 0.01% or 0.5% to about 95% or 99% by weight of the composition. The pharmaceutical compositions are prepared by any of the well-known techniques of pharmacy including, but not limited to, admixing the components, optionally including one or more accessory ingredients.

The pharmaceutical compositions of this invention include those suitable for oral, rectal, topical, inhalation (e.g., via an aerosol) buccal (e.g., sub-lingual), vaginal, parenteral (e.g., subcutaneous, intramuscular, intradermal, intraarticular, intrapleural, intraperitoneal, intracerebral, intraarterial, or intravenous), topical (i.e., both skin and mucosal surfaces, including airway surfaces) and transdermal administration, although the most suitable route in any given case will depend, as is well known in the art, on such factors as the species, age, gender and overall condition of the subject, the nature and severity of the condition being treated and/or on the nature of the particular composition (i.e., dosage, formulation) that is being administered. Pharmaceutical compositions suitable for oral administration can be presented in discrete units, such as capsules, cachets, lozenges, or tablets, each containing a predetermined amount of the composition of this invention; as a powder or granules; as a solution or a suspension in an aqueous or non-aqueous liquid; or as an oil-in-water or water-in-oil emulsion. Oral delivery can be performed by complexing a composition of the present invention to a carrier capable of withstanding degradation by digestive enzymes in the gut of an animal. Examples of such carriers include plastic capsules or tablets, as known in the art. Such formulations are prepared by any suitable method of pharmacy, which includes the step of bringing into association the composition and a suitable carrier (which may contain one or more accessory ingredients as noted above). In general, the pharmaceutical composition according to embodiments of the present invention are prepared by uniformly and intimately admixing the composition with a liquid or finely divided solid carrier, or both, and then, if necessary, shaping the resulting mixture. For example, a tablet can be prepared by compressing or molding a powder or granules containing the composition, optionally with one or more accessory ingredients. Compressed tablets are prepared by compressing, in a suitable machine, the composition in a free-flowing form, such as a powder or granules optionally mixed with a binder, lubricant, inert diluent, and/or surface active/dispersing agent(s). Molded tablets are made by molding, in a suitable machine, the powdered compound moistened with an inert liquid binder.

Pharmaceutical compositions suitable for buccal (sub-lingual) administration include lozenges comprising the composition of this invention in a flavored base, usually sucrose and acacia or tragacanth; and pastilles comprising the composition in an inert base such as gelatin and glycerin or sucrose and acacia.

Pharmaceutical compositions of this invention suitable for parenteral administration can comprise sterile aqueous and non-aqueous injection solutions of the composition of this invention, which preparations are preferably isotonic with the blood of the intended recipient. These preparations can contain anti-oxidants, buffers, bacteriostats and solutes, which render the composition isotonic with the blood of the intended recipient. Aqueous and non-aqueous sterile suspensions, solutions and emulsions can include suspending agents and thickening agents. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

The compositions can be presented in unit dose or multi-dose containers, for example, in sealed ampoules and vials, and can be stored in a freeze-dried (lyophilized) condition requiring only the addition of the sterile liquid carrier, for example, saline or water-for-injection immediately prior to use. Extemporaneous injection solutions and suspensions can be prepared from sterile powders, granules and tablets of the kind previously described. For example, an injectable, stable, sterile composition of this invention in a unit dosage form in a sealed container can be provided. The composition can be provided in the form of a lyophilizate, which can be reconstituted with a suitable pharmaceutically acceptable carrier to form a liquid composition suitable for injection into a subject. The unit dosage form can be from about 1 μg to about 10 grams of the composition of this invention. When the composition is substantially water-insoluble, a sufficient amount of emulsifying agent, which is physiologically acceptable, can be included in sufficient quantity to emulsify the composition in an aqueous carrier. One such useful emulsifying agent is phosphatidyl choline.

Pharmaceutical compositions suitable for rectal administration are preferably presented as unit dose suppositories. These can be prepared by admixing the composition with one or more conventional solid carriers, such as for example, cocoa butter and then shaping the resulting mixture.

Pharmaceutical compositions of this invention suitable for topical application to the skin preferably take the form of an ointment, cream, lotion, paste, gel, spray, aerosol, or oil. Carriers that can be used include, but are not limited to, petroleum jelly, lanoline, polyethylene glycols, alcohols, transdermal enhancers, and combinations of two or more thereof. In some embodiments, for example, topical delivery can be performed by mixing a pharmaceutical composition of the present invention with a lipophilic reagent (e.g., DMSO) that is capable of passing into the skin.

Pharmaceutical compositions suitable for transdermal administration can be in the form of discrete patches adapted to remain in intimate contact with the epidermis of the subject for a prolonged period of time. Compositions suitable for transdermal administration can also be delivered by iontophoresis (see, for example, Pharmaceutical Research 3:318 (1986)) and typically take the form of an optionally buffered aqueous solution of the composition of this invention. Suitable formulations can comprise citrate or bistris buffer (pH 6) or ethanol/water and can contain from 0.1 to 0.2M active ingredient.

An effective amount of a composition of this invention will vary from composition to composition and subject to subject, and will depend upon a variety of factors such as age, species, gender, weight, overall condition of the subject and the particular disease or disorder to be treated. An effective amount can be determined in accordance with routine pharmacological procedures know to those of skill in the art. In some embodiments, a dosage ranging from about 0.1 μg/kg to about 1 gm/kg will have therapeutic efficacy. In embodiments employing viral vectors for delivery of the gene editing system of this invention, viral doses can be measured to include a particular number of virus particles or plaque forming units (pfu) or infectious particles, depending on the virus employed. For example, in some embodiments, particular unit doses can include about 10³, 10⁴, 10⁵, 10⁶, 10⁷, 10⁸, 10⁹, 10¹¹, 10¹¹, 10¹², 10¹³, 10¹⁴, 10¹⁵, 10¹⁶, 10¹⁷ or 10¹⁸ pfu or infectious particles.

The frequency of administration of a composition of this invention can be as frequent as necessary to impart the desired therapeutic effect. For example, the composition can be administered one, two, three, four or more times per day, one, two, three, four or more times a week, one, two, three, four or more times a month, one, two, three or four times a year and/or as necessary to control a particular condition and/or to achieve a particular effect and/or benefit. In some embodiments, one, two, three or four doses over the lifetime of a subject can be adequate to achieve the desired therapeutic effect. The amount and frequency of administration of the composition of this invention will vary depending on the particular condition being treated or to be prevented and the desired therapeutic effect.

In one embodiment, the oligonucleotide that binds the regulatory sequence is repeatedly administered to a subject over a given period of time (e.g., the lifetime of the subject, or the duration of the disease). For example, the oligonucleotide that binds the regulatory sequence can be administered one, two, three, four or more times per day, one, two, three, four or more times a week, one, two, three, four or more times a month, one, two, three or four times a year and/or as necessary to control a particular condition and/or to achieve a particular effect and/or benefit.

The components of the composition (e.g., (a) a vector comprising a nucleic acid sequence encoding a nuclease, (b) an oligonucleotide that binds to the regulatory sequence) can be administered to the subject at substantially the same time. Alternatively, the components can be administered at different time, for example, (a) can be administered at least an hour, at least a day, at least a week, at least a month, at least a year after, or prior to, the administration of (b).

The components of the composition (e.g., (a) a vector comprising a nucleic acid sequence encoding a CRISPR-associated nuclease, (b) a gRNA that binds to the target gene sequence, and (c) an oligonucleotide that binds to the regulatory sequence) can be administered to the subject at substantially the same time. Alternatively, the components can be administered at different time, for example, (a) and (b) can be administered at substantially the same time, and (c) can be administered at least an hour, at least a day, at least a week, at least a month, at least a year after the administration of (a) and (b).

The components of the gene editing system described herein need not be administered at the same frequency, intervals, and/or levels. It is specifically contemplated herein that each component be administered at the frequency, interval, and/or level that results in the desired therapeutic effect.

The compositions of this invention can be administered to a cell of a subject either in vivo or ex vivo. For administration to a cell of the subject in vivo, as well as for administration to the subject, the compositions of this invention can be administered, for example as noted above, orally, parenterally (e.g., intravenously), by intramuscular injection, intradermally (e.g., by gene gun), by intraperitoneal injection, subcutaneous injection, transdermally, extracorporeally, topically or the like. Also, the composition of this invention can be pulsed onto dendritic cells, which are isolated or grown from a subject's cells, according to methods well known in the art, or onto bulk PBMC or various cell subtractions thereof from a subject.

If ex vivo methods are employed, cells or tissues can be removed and maintained outside the body according to standard protocols well known in the art while the compositions of this invention are introduced into the cells or tissues. For example, the gene editing system of this invention can be introduced into cells via any gene transfer mechanism, such as, for example, virus-mediated gene delivery, calcium phosphate mediated gene delivery, electroporation, microinjection or proteoliposomes. The transduced and/or transfected cells can then be infused (e.g., in a pharmaceutically acceptable carrier) or transplanted back into the subject per standard methods for the cell or tissue type. Standard methods are known for transplantation or infusion of various cells into a subject.

Formulations of the present invention may comprise sterile aqueous and non-aqueous injection solutions of the active compound, which preparations are preferably isotonic with the blood of intended recipient and essentially pyrogen free. These preparations may contain anti-oxidants, buffers, bacteriostats and solutes, which render the formulation isotonic with the blood of the intended recipient. Aqueous and non-aqueous sterile suspensions may include suspending agents and thickening agents. The formulations may be presented in unit dose or multi-dose containers, for example, sealed ampoules and vials, and may be stored in a freeze-dried (lyophilized) condition requiring only the addition of the sterile liquid carrier, for example, saline or water-for-injection immediately prior to use.

The components described herein (e.g., (a) a vector comprising a nucleic acid sequence encoding a nuclease, (b) an oligonucleotide that binds to the regulatory sequence) can be formulated into the same composition (e.g., one composition having all components). Alternatively, the components can be formulated into two different compositions.

The components described herein (e.g., (a) a vector comprising a nucleic acid sequence encoding a CRISPR-associated nuclease, (b) a gRNA that binds to the target gene sequence, and (c) an oligonucleotide that binds to the regulatory sequence) can be formulated into the same composition (e.g., one composition having all components). Alternatively, the components can formulated into different compositions, for example, (a) and (b) are formulated into one composition, and (c) is formulated into a different composition; or (a), (b), and (c) are all formulated in different compositions.

In one formulation, the components of the gene editing system of this invention may be delivered or introduced to the subject as naked DNA.

In one formulation, the components of the gene editing system of this invention may be contained within a lipid particle or vesicle, such as a liposome or microcrystal, which may be suitable for parenteral administration. The particles may be of any suitable structure, such as unilamellar or plurilamellar, so long as the compound is contained therein. Positively charged lipids such as N-[1-(2,3-dioleoyloxi)propyll-N,N,N-trimethyl-ammoniummethylsulfate, or “DOTAP,” are particularly preferred for such particles and vesicles. The preparation of such lipid particles is well known. See, e.g., U.S. Pat. No. 4,880,635 to Janoff et al.; U.S. Pat. No. 4,906,477 to Kurono et al.; U.S. Pat. No. 4,911,928 to Wallach; U.S. Pat. No. 4,917,951 to Wallach; U.S. Pat. No. 4,920,016 to Allen et al.; U.S. Pat. No. 4,921,757 to Wheatley et al.; etc. In one formulation, the gene editing system of this invention may be contained within a nanoparticle. In another formulation, the gene editing system of this invention may be contained within a recombinant AAV capsid.

In one embodiment, component (c) is delivered or introduced to the subject via naked DNA, or within a lipid particle, a nanoparticle, or a recombinant AAV capsid.

The pharmaceutical compositions of this invention can be used, for example, in the production of a medicament for the treatment of a disease and/or disorder as described herein.

The Following Sequences are Included in the Present Invention:

SEQ ID NO:1. plasmid TRCBA-int-luc mut. Nts 163-2036: CBA promoter; nts. 2739-4573: mutant intron (654 C-T); nts 4592-4813: polyA signal.

SEQ ID NO:2. plasmid TRCBA-int-luc (wt). Nts 163-2036: CBA promoter; nts. 2739-3588: wt intron (654 C); nts 2071-4573: intron in luciferase; nts 4592-4813: polyA signal.

SEQ ID NO:3. plasmid TRCBA-int-luc (657GT). Nts 163-2036: CBA promoter; nts. 2739-3588: mutant intron (654 C-T; 657 TA-GT); nts 2071-4573: intron in luciferase; nts 4592-4813: polyA signal.

SEQ ID NO:4. plasmid GL3-int-Luc (mut). Nts 48-250: SV40 promoter; nts. 948-1797: mutant intron (654 C-T); nts 2814-3035: polyA signal; nts. 280-2782: luciferase with mutant intron. WO 2006/119137 PCT/US2006/016514

SEQ ID NO:5. plasmid GL3-int-Luc (wt). Nts 48-250: SV40 promoter; nts. 948-1797: wt intron (654 C); nts 280-2782: luciferase with intron; nts 2814-3035: polyA signal.

SEQ ID NO:6. plasmid GL3-int-Luc (657GT). Nts 48-250: SV40 promoter; nts. 948-1797: intron (654 C-T; 657TA-GT); nts 280-2782: luciferase with mutant intron; nts 2814-3035: polyA signal.

SEQ ID NO:7. plasmid GL3-2int-fron-sph (mut). Nts 48-250: SV40 promoter; nts. 251-1100; 1771-2620: mutant introns (654 C-T); nts 1103-3635: luciferase with mutant intron; nts 3637-3858: polyA signal.

SEQ ID NO:8. plasmid GL3-3int-2fron-sph (mut). Nts 48-250: SV40 promoter; nts. 251-1100; 1106-1965; 2635-3484: mutant introns (654 C-T); nts 1967-4469: luciferase with mutant intron; nts 4514-4735: polyA signal.

SEQ ID NO:9. plasmid GL3-int-luc A (mut). Nts 48-250: SV40 promoter; nts. 673-1522: intron (654 C-T); nts 280-2782: luciferase with intron; nts 2814-3035: polyA signal.

SEQ ID NO:10. plasmid GL3-int-Luc B (mut). Nts 48-250: SV40 promoter; nts. 1440-2289: intron (654 C-T); nts 280-2782: luciferase with intron; nts 2814-3035: polyA signal.

SEQ ID NO:11. plasmid GL3-int-Luc C (mut). Nts 48-250: SV40 promoter; nts. 1691-2540: intron (654 C-T); nts 280-2782: luciferase with intron; nts 2814-3035: polyA signal.

SEQ ID NO:12. plasmid GL3-int-fron (mut). Nts 48-250: SV40 promoter; nts. 251-1100: intron (654 C-T); nts 1103-2755: luciferase with intron; nts 2787-3008: polyA signal.

SEQ ID NO:13. plasmid GL3-2int-sph (mut). Nts 48-250: SV40 promoter; nts. 948-1797; 1798-2647: intron (654 C-T); nts 280-3632: luciferase with intron; nts 3664-3885: polyA signal.

SEQ ID NO:14. plasmid GL3-2int-sph C (mut). Nts 48-250: SV40 promoter; nts. 948-1797; 2541-3390: intron (654 C-T); nts 280-3632: luciferase with intron; nts 3664-3885: polyA signal.

SEQ ID NO:15. plasmid GL3-sint200-sph (mut). Nts 48-250: SV40 promoter; nts. 948-1597: intron (654 C-T); nts 280-2582: luciferase with intron; nts 2794-2835: polyA signal.

SEQ ID NO:16. plasmid GL3-sint200-sph (657 GT). Nts 48-250: SV40 promoter; nts. 948-1597: intron (654 C-T; 657 TA-GT); nts 280-2582: luciferase with intron; nts 2794-2835: polyA signal.

SEQ ID NO:17. plasmid GL3-sint425-sph. Nts 48-250: SV40 promoter; nts. 948-1373: intron (654 C-T); nts 280-2358: luciferase with intron; nts 2569-2615: polyA signal.

SEQ ID NO:18. mutant intron (654 C-T).

SEQ ID NO:19. wt intron (654 C).

SEQ ID NO:20. intron with two mutations (654 C-T; 657 TA-GT).

SEQ ID NO:21. luciferase cDNA with mutant intron (654 C-T) at nts. 669-1518.

SEQ ID NO:22. luciferase cDNA with wild type intron at nts. 669-1518.

SEQ ID NO:23. luciferase cDNA with double mutant intron (C654 C-T; 657 TA-GT) at nts. 669-1518.

SEQ ID NO:24. luciferase cDNA with mutant intron (654 C-T) at nts. 1-850 and mutant intron (654 C-T) at nts. 1521-2370.

SEQ ID NO:25. luciferase cDNA with mutant intron (654 C-T) at nts. 1-850 and two mutant introns (654 C-T) at nts. 861-1710 and nts. 2385-3234.

SEQ ID NO:26. luciferase cDNA with mutant intron (654 C-T) at alternative location A (nts. 394-1243).

SEQ ID NO:27. luciferase cDNA with mutant intron (654 C-T) at alternative location B (nts. 1161-2010).

SEQ ID NO:28. luciferase cDNA with mutant intron (654 C-T) at alternative location C (nts. 1412-2261).

SEQ ID NO:29. luciferase cDNA with mutant intron (654 C-T) upstream of translation site (nts. 1-850).

SEQ ID NO:30. luciferase cDNA with two mutant introns (654 C-T): at nts. 669-1518 and at nts. 1519-2368.

SEQ ID NO:31. luciferase cDNA with two mutant introns (654 C-T): at nts. 669-1518 and at nts. 2262-3111.

SEQ ID NO:32. luciferase cDNA with mutant intron (654 C-T) at nts. 669-1318 and 200 base pair deletion.

SEQ ID NO:33. luciferase cDNA with double mutant intron (654 C-T; 657 TA-GT) at nts. 669-1318 and 200 basepair deletion.

SEQ ID NO:34. luciferase cDNA with mutant intron (654 C-T) at nts. 669-1094 and 425 basepair deletion.

SEQ ID NO:35. plasmid TRCBA with alpha antitrypsin cDNA and mutant intron (654 C-T) at nts. 2866-3715.

SEQ ID NO:36. alpha antitrypsin cDNA with mutant intron (654 C-T) at nts. 772-1621.

SEQ ID NO:37. oligonucleotide that binds the regulatory sequence GCT ATT ACC TTA ACC CAG for IVS2-654.

SEQ ID NO: 38. oligonucleotide that binds the regulatory sequence GCA CTT ACC TTA ACC CAG for IVS2-654 with 657GT mutation).

SEQ ID NO:50 (IVS2-654 intron with 564CT mutation).

SEQ ID NO:51 (IVS2-654 intron with 657G mutation).

SEQ ID NO:52 (IVS2-654 intron with 658T mutation).

SEQ ID NO:20 (IV S2-654 intron with 657GT mutation).

SEQ ID NO:53 (IVS2-654 intron with 200 bp deletion).

SEQ ID NO:54 (IVS2-654 intron with 425 bp deletion).

SEQ ID NO:68 (IVS2-654 intron with only 197 bp).

SEQ ID NO:69 (IVS2-654 intron with only 247 bp).

SEQ ID NO:55 (IVS2-654 intron with 6A mutation).

SEQ ID NO:56 (IVS2-654 intron with 564C mutation).

SEQ ID NO:57 (IVS2-654 intron with 841A mutation).

SEQ ID NO:58 (IVS2-705 intron).

SEQ ID NO:59 (TVS2-705 intron with 564CT mutation).

SEQ ID NO:60 (IVS2-705 intron with 657G mutation).

SEQ ID NO:61 (IVS2-705 intron with 658T mutation).

SEQ ID NO:62 (IVS2-705 intron with 657GT mutation).

SEQ ID NO:63 (TVS2-705 intron with 200 bp deletion).

SEQ ID NO:64 (IVS2-705 intron with 425 bp deletion).

SEQ ID NO:65 (IVS2-705 intron with 6A mutation).

SEQ ID NO:66 (IVS2-705 intron with 564C mutation).

SEQ ID NO:67 (IVS2-705 intron with 841A mutation).

SEQ ID NO:70 (CFTR exon 19 wild-type sequence).

SEQ ID NO:71 (CFTR exon 19 3849+10 kb C-to-T mutation).

SEQ ID NO:72 (CFTR exon 19 wild-type oligo).

SEQ ID NO:73 (CFTR exon 19 3849+10 kb C-to-T mutation oligo).

SEQ ID NO:74 (Mouse dystrophin intron 22, exon 23 and intron 23 wild-type sequence).

SEQ ID NO:75 (mdx Mouse dystrophin intron 22, exon 23 and intron 23 nonsense mutation).

SEQ ID NO:76 (Antisense exon 23 skipping inducing oligo).

SEQ ID NO:39 (oligo for 6A mutation in IVS2-654).

SEQ ID NO:40 (oligo for 564C mutation in IVS2-654).

SEQ ID NO:41 (oligo for 564CT mutation in IVS2-654).

SEQ ID NO:43 (oligo for 841A mutation in IVS2-654).

SEQ ID NO:44 (oligo for 657G mutation in IVS2-654).

SEQ ID NO:45 (oligo for 658T mutation in IVS2-654).

SEQ ID NO:42 (oligo for 705G mutation in IVS2-705).

SEQ ID NO:49 (oligo for IVS2-705).

SEQ ID NO:46 (oligo for IVS2-654).

SEQ ID NO:47 (oligo for IVS2-654).

SEQ ID NO:48 (oligo for IVS2-654).

All publications, patent applications, patents, patent publications and other references cited herein are incorporated by reference in their entireties for the teachings relevant to the sentence and/or paragraph in which the reference is presented. The examples, which follow, are set forth to illustrate the present invention, and are not to be construed as limiting thereof.

The present invention can be further described in the following numbered paragraphs:
1. A system for editing a gene (e.g., altering expression of at least one gene product) having reduced off target effects comprising introducing into a cell having a target gene sequence
a) a vector comprising a nucleic acid sequence encoding a nuclease, wherein the nucleic acid encoding the nuclease contains within its sequence a regulatory nucleic acid sequence having a first and second set of splice elements defining a first and second intron, wherein the first and second intron flank a sequence encoding a non-naturally occurring exon sequence containing an in-frame stop codon sequence, and wherein the first and second intron are spliced from the pre-mRNA message to produce an mRNA encoding a non-functional nuclease that contains an amino acid sequence encoded by the non-naturally occurring exon; and
b) an oligonucleotide that binds to the regulatory nucleic acid sequence, wherein within the cell the oligonucleotide prevents splicing of the second set of splice elements from the mRNA, thereby producing an mRNA that lacks the exon and encodes a nuclease that is functional for gene editing of a target gene.
2. The system of paragraph 1, wherein the nuclease is selected from the group consisting of a CRISPR-associated nuclease, a meganuclease, a zinc finger nuclease, and a transcription activator-like effector nuclease.
3. The system of paragraph 1, wherein the nuclease is an endonuclease or an exonuclease.
4. The system of any preceding paragraph, wherein component (a) further comprises a gRNA that binds to the sequence of the target gene.
5. The system of any preceding paragraph, wherein the regulatory nucleic acid sequence is a beta-globin mutant intron.
6. The system of any preceding paragraph, comprising at least two regulatory nucleic acid sequences.
7. The system of any preceding paragraph, wherein the regulatory nucleic acid sequence comprises a sequence selected from the group consisting of: SEQ ID NO: 18 (IVS2-654 intron C-T), SEQ ID NO:50 (IVS2-654 intron with 564CT mutation), SEQ ID NO:51 (IVS2-654 intron with 657G mutation), SEQ ID NO:52 (IVS2-654 intron with 658T mutation), SEQ ID NO:20 (IVS2-654 intron with 657GT mutation), SEQ ID NO:53 (IVS2-654 intron with 200 by deletion), SEQ ID NO:68 (IVS2-654 intron with only 197 bp), SEQ ID NO:55 (IVS2-654 intron with 6A mutation), SEQ ID NO:56 (IVS2-654 intron with 564C mutation), SEQ ID NO:57 (IVS2-654 intron with 841A mutation), SEQ ID NO:59 (IVS2-705 intron with 564CT mutation), SEQ ID NO:60 (IVS2-705 intron with 657G mutation), SEQ ID NO:61 (IVS2-705 intron with 658T mutation), SEQ ID NO:62 (IVS2-705 intron with 657GT mutation), SEQ ID NO:63 (IVS2-705 intron with 200 by deletion), SEQ ID NO:64 (IVS2-705 intron with 425 by deletion), SEQ ID NO:65 (IVS2-705 intron with 6A mutation), SEQ ID NO:66 (IVS2-705 intron with 564C mutation), SEQ ID NO:67 (IVS2-705 intron with 841A mutation). SEQ ID NO: 74, SEQ ID NO:75, SEQ ID NO: 76, SEQ ID NO: 77, SEQ ID NO:78, SEQ ID NO: 143, SEQ ID NO: 144, SEQ ID NO: 145, SEQ ID NO: 146, SEQ ID NO: 147, SEQ ID NO: 148; and in any combination thereof, including singly.
8. The system of any preceding paragraph, wherein the oligonucleotide that binds to the regulatory sequence comprises a sequence selected from the group consisting of: SEQ ID NO:37 (oligo for IVS2-654 CT), SEQ ID NO:38 (oligo for IVS2-654 with 657GT mutation), SEQ ID NO:39 (oligo for 6A mutation in IVS2-654), SEQ ID NO:40 (oligo for 564C mutation in IVS2-654), SEQ ID NO:41 (oligo for 564CT mutation in IVS2-654), SEQ ID NO:43 (oligo for 841A mutation in IVS2-654), SEQ ID NO:44 (oligo for 657G mutation in IVS2-654), SEQ ID NO:45 (oligo for 658T mutation in IVS2-654), SEQ ID NO:42 (oligo for 705G mutation in IVS2-705). SEQ ID NO:49 (oligo for IVS2-705), SEQ ID NO:76 (Antisense exon 23 skipping inducing oligo) respectively, and SEQ ID NO 138 (Oligo for LUC-AON1), SEQ ID NO: 139 (oligo for LUC-AON2), SEQ ID NO: 140 (Oligo for LUC-AON3), SEQ ID NO: 141 (Oligo for LUC-AON4), SEQ ID NO: 142 (Oligo for IVS2(S0)-654, LUC-654) and SEQ ID NO: 149 (Oligo for WT regulatory).
9. The system of any preceding paragraph, wherein the off-target effects are reduced by at least 30%.
10. The system of any preceding paragraph, wherein the off-target effects are reduced by at least 40%, at least 50%, at least 60%, at least 70%, at least 80%, or at least 90% or more.
11. The system of any preceding paragraph, wherein components (a) and (b) are located on same or different vectors.
12. The system of any preceding paragraph, wherein component (b) is introduced to cell as naked DNA.
13. The system of any preceding paragraph, wherein component (b) is introduced to cell using a lipid formulation.
14. The system of any preceding paragraph, wherein component (b) is introduced to cell using a nanoparticle.
15. The system of any preceding paragraph, wherein component (b) is administered at a time point following the administration of (a).
16. The system of any preceding paragraph, wherein components (a) and (b) are administered at substantially the same time.
17. The system of any preceding paragraph, wherein the expression of (a) is not detected in the cell in the absence of (b), or absence of expression of (b).
18. The system of any preceding paragraph, wherein the expression of (a) is dependent on the expression of (b).
19. The system of any preceding paragraph, wherein component (b) controls an “ON” and/or “OFF” status of the system.
20. The system of paragraph 19, wherein the “ON” and/or “OFF” status is under selective control.
21. The system of paragraph 20, wherein the selective control is spatial and/or temporal control.
22. The system of any preceding paragraph, wherein the vector is a viral vector.
23. The system of paragraph 22, wherein the viral vector is selected form the group consisting of: from the group consisting of an AAV vector, an adenovirus vector, a lentivirus vector, a retrovirus vector, a herpesvirus vector, an alphavirus vector, a poxvirus vector, a baculovirus vector and a chimeric virus vector.
24. The system of any preceding paragraph, wherein the vector is a non-viral vector.
25. The system of any preceding paragraph, wherein the nuclease is a CRISPR-associated nuclease.
26. The system of any preceding paragraph, wherein the CRISPR-associated nuclease creates double stand breaks for gene editing and wherein the CRISPR-associated nuclease is selected from the group consisting of Cpf1, C2c1, C2c3, Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, C2c1, C2c3, Cas12a, Cas12b, Cas12c, Cas12d, Cas12e, Cas13a, Cas13b, and Cas13c.
27. The system of any preceding paragraph, wherein the CRISPR-associated nuclease is a Cas9 variant selected from Staphylococcus aureus (SaCas9), Streptococcus thermophilus (StCas9), Neisseria meningitidis (NmCas9), Francisella novicida (FnCas9), and Campylobacter jejuni (CjCas9).
28. The system of any preceding paragraph, wherein the CRISPR-associated nuclease has been modified for gene-editing without double strand DNA breaks (such as CRISPRi or CRISPRa) and is selected from the group consisting of dCas, nCas, and Cas 13.
29. The system of any preceding paragraph, wherein the CRISPR-associated nuclease is codon optimized for expression in the eukaryotic cell.
30. The system of any preceding paragraph, wherein the gene editing is decreasing the expression of one or more gene products.
31. The system of any preceding paragraph, wherein the gene editing is increasing expression of one or more gene products.
32. The system of any preceding paragraph, wherein the cell is a mammalian or human cell.
33. The system of any preceding paragraph, wherein the cell is in vivo.
34. The system of any preceding paragraph, wherein the cell is in vitro.
35 The system of any preceding paragraph, wherein the target gene is a disease gene.
36. A method for editing a gene in a subject, the method comprising administering the system of paragraphs 1-35 to a subject in need of gene editing.

### EXAMPLES

### Example 1. Differential Regulation of Multiple Transgenes in AAV Vectors by Alternative Splicing

### Introduction

Wild type AAV is a non-pathogenic, non-enveloped, small single-stranded DNA virus with a genome of 4.7 kilobases (kb). Recombinant AAV has been developed and applied as a gene therapy vector for decades. The ability to regulate the expression of transgene is essential to ensure the safety of many gene therapy strategies. Several strategies of controlling transgene expression like the tet-on, or the rapamycin-inducible system have been tested for gene transfer mediated by AAV vector. Each regulation system has advantages and disadvantages depend on the target to treatment. As a strategy to develop the transgene regulation system that simplifying the gene delivery system, eliminating the possibility of immune response against the transactivator protein, and inducing multiple transgene individually, and more importantly maximizing the packaging capacity of AAV vectors, splice switching mechanism of IVS2-654 intron was adapted into the AAV mediated gene delivery.

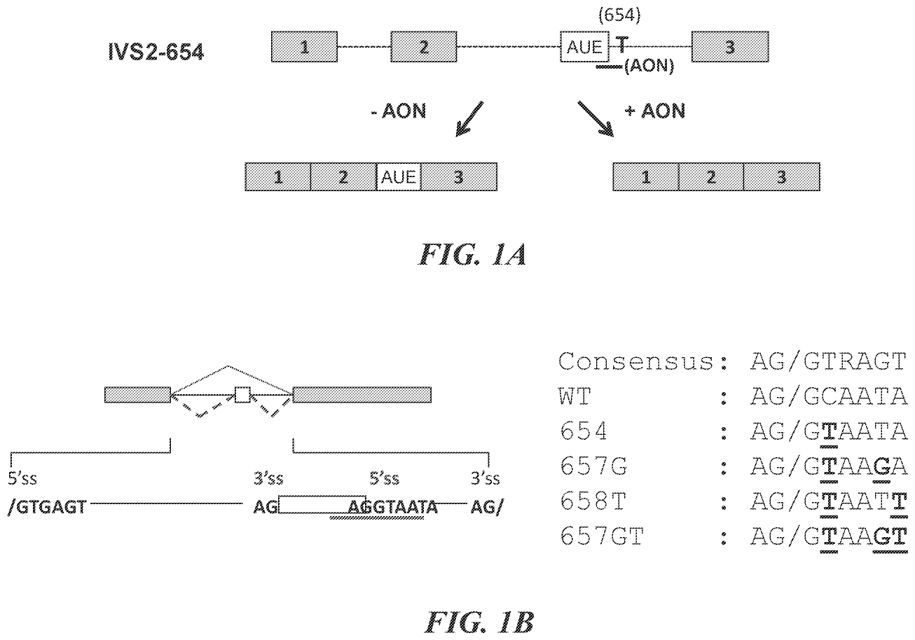

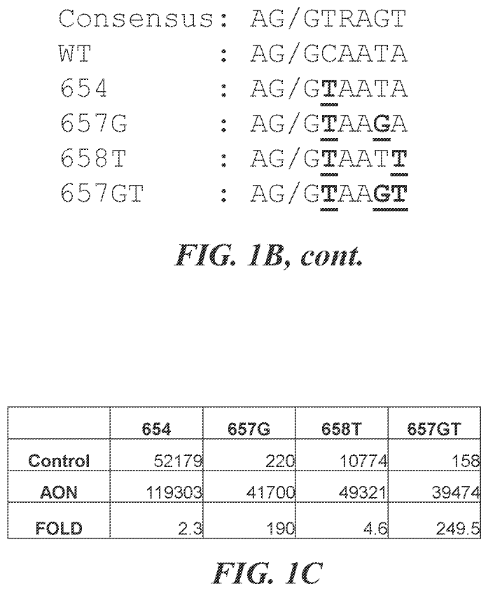

It has been known over 90% of transcripts which contain multiple exons undergo alternative splicing. In these conditions, splice site selection is a one of critical factors to determine gene expression. It has been reported that many cases of genetic disease are caused by mutations which alter the splicing pattern. In past decades, the usage of antisense oligonucleotide (AON) has been intensively studied and applied in vitro and in vivo as a therapeutic agent that can control the gene expression by restore or alter the splicing. One of the first targets to restore functional gene expression by splice switching using AON was thalassemic mutation of the β-globin gene. The second intron of the β-globin transcript, IVS2, contains consensus 5′ and 3′ splice sites and this intron is constitutively removed during the splicing process to produce functional protein in normal condition. A nucleotide change C to T at 654 of IVS2, which is one of the frequently found mutations among thalassemic patients, generate an aberrant 5′ splice site at 653 with a cryptic 3′ splice site and alternatively used exon (AUE) upstream (FIG. 1A). These cryptic splice sites are preferably used by splicing machinery followed by retention of AUE in β-globin mRNA which shifted the open reading frame downstream and generated truncated protein. This aberrant splicing could be restored by administration of AON which bind to and block the usage of the cryptic 5′ splice site (FIG. 1A). In a recent publication, the inventor showed this inducible system using IVS2-654 mutant intron and corresponding AON can be used to control the transgenes mediated by AAV in vitro and in vivo.

The ability to regulate the expression of transgene is essential to ensure the safety of many gene therapy strategies. This is particularly the case for gene therapy of eye diseases due to neovascular disorders, which may require long-term presence of multiple angiostatic proteins that could inhibit normal as well as abnormal blood vessels. In theory, current regulation systems could be combined to regulate multiple transgenes. However, due to the requirements of the systems, such an approach would be very cumbersome. Therefore, alternative splicing was developed as a strategy to independently control the expression of multiple transgenes in the same organism. In the regulation system described herein, which is based on alternative splicing, transgene expression is controlled by using AON targeting the 5′ alternative splice site to modulate the alternative splicing of transgene message. In a previous study, the inventor successfully used LNA654, a 16-mer oligonucleotide complementary to both the 5′ alternative splice site and its flanking sequences to induce transgene expression. In this system, splicing switch can be determined by the specificity of the AON. Modified AON, LNA has high specificity toward their targets. Their specificity can be distinguished by a few nucleotide differences. This ability is a great advantage for multiple gene regulation. Only a few altered nucleotides of flanking region of alternatively used 5′ donor site in the intron can be another distinguishable target. Therefore, their ability to control multiple genes individually by a few altered nucleotides of their target region can be applied without backbone change. It would be possible to use different targeting AONs to independently control the expression of multiple transgenes in the same living organism. This idea would allow a single patient to receive multiple gene therapy treatments requiring differential regulation of transgene expression.

Herein, it is reported that this inducible system is significantly improved for tight and efficient regulation by optimizing intron size and splice site. This optimized system demonstrated significantly improved induction of transgenes in vitro and in vivo. In addition, transgene expression can be re-induced by re-administration of AON in mouse eyes. It is also shown herein that this system could be used for differential regulation of multiple transgenes using a set of modified introns with their corresponding AONs.

### Results

Optimization of Alternatively Used 5′ Splice Site of IVS2-654 Intron for Efficient Regulation.

To facilitate the optimization of the alternative splicing for controlling transgene expression, the firefly luciferase marker gene was used for the insertion of the 850 bp alternatively spliced intron IVS2-654. Thus, control of transgene expression could be conveniently determined by assaying the levels of luciferase expression under the conditions for both AUE inclusion and AUE skipping, in the presence or absence of the AON. First, the alternative splicing for controlling transgene expression was optimized by modifying the alternative splice site of the IVS2-654 intron. Nucleotide sequences at 657 and 658 of IVS2-654 intron, which are the 5^{th} and 6^{th} downstream nucleotides of the alternative 5′ splice site, are T and A. These are less consensus compared to those of the consensus 5′ splice site G and T. The T at nucleotide 657 was converted to G, A at 658 to T, or both the TA to GT. The mutations were to increase the strength of the alternative 5′ splice site by making the splice site more similar or identical to the consensus sequences (FIG. 1B). The resulting plasmids and corresponding AONs were transfected into 293 cells using the PEI transfection method. Twenty-four hours after the transfection, the cells were harvested for quantification of luciferase expression. Construct 658T yielded an approximate two-fold improvement in the induction levels compared to construct IVS2-654. Consequently, constructs 657G and 657GT resulted in a 190- and 250-fold improvement in the level of induction (FIG. 1C). The increase in the level of induction was apparently due to more dramatic decrease in the background level of transgene expression than in the induced level of transgene expression. These results indicated that by modulating the strength of the splice site, alternative splicing could be optimized to control transgene expression.

Optimization of IVS2-654 Intron Size to Maximize Transgene Capacity of AAV.

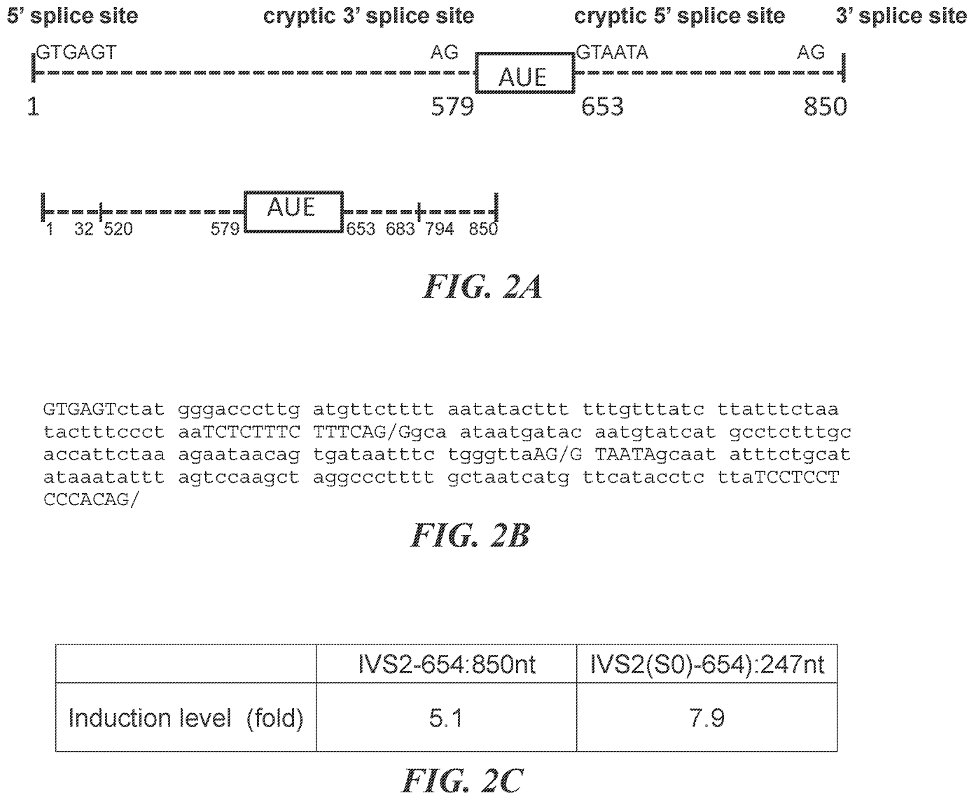

AAV has packaging limitations of 4.7 kb because it allows only around 3 kb in maximum size for the transgene coding region depending on the size of the promoter, poly A, and ITR. The original IVS2-654 intron is 850 nucleotides (nt) long (FIG. 2A), and insertion of this intron into the open reading frame (ORF) of the transgene for regulation further reduces cloning capacity for the transgene. Therefore, the 850 nt IVS2-654 was converted to a small intron of 247 nt, termed S0, which contained the essential splice sites and the AUE as well as the first 32 nt on the 5′ end and the last 57 nt on the 3′ end that are required for the efficient splicing of the β-globin mRNA (FIG. 2B). Insertion of the S0 intron into the luciferase gene, yielding construct IVS2 (S0)-654, resulted in alternative splicing of the message. Importantly, the induction level by AON for the small intron was similar to that of original IVS2-654 intron (FIG. 2C).

Individual Regulation of the Luciferase Expression of Modified Intron Containing Constructs by their Corresponding AONs.

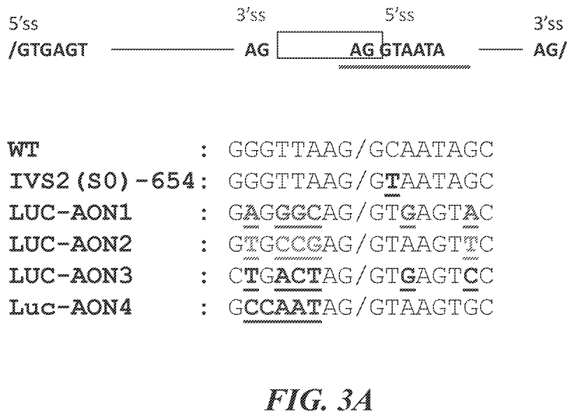

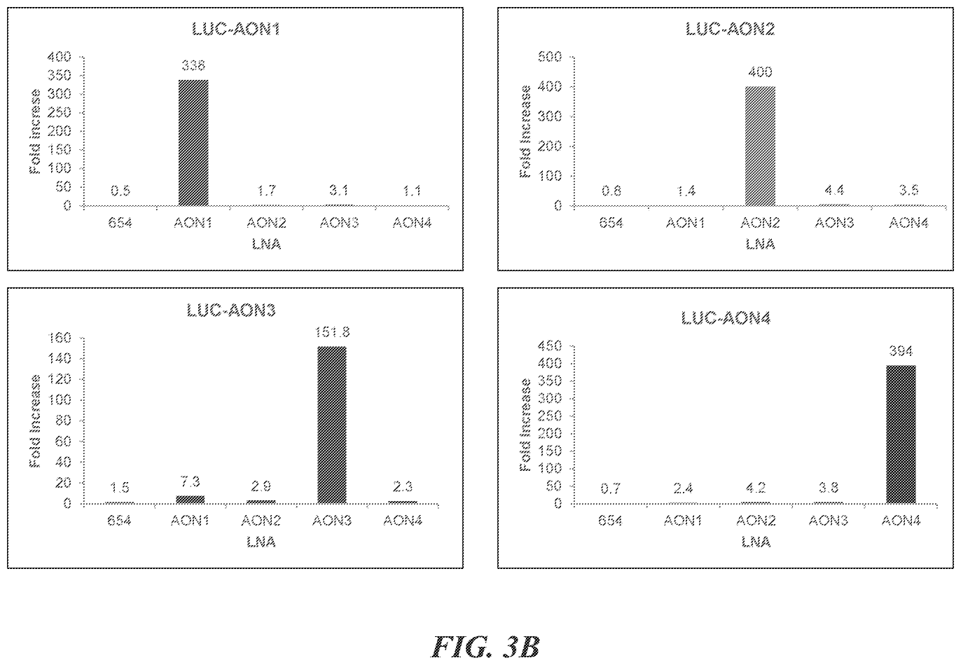

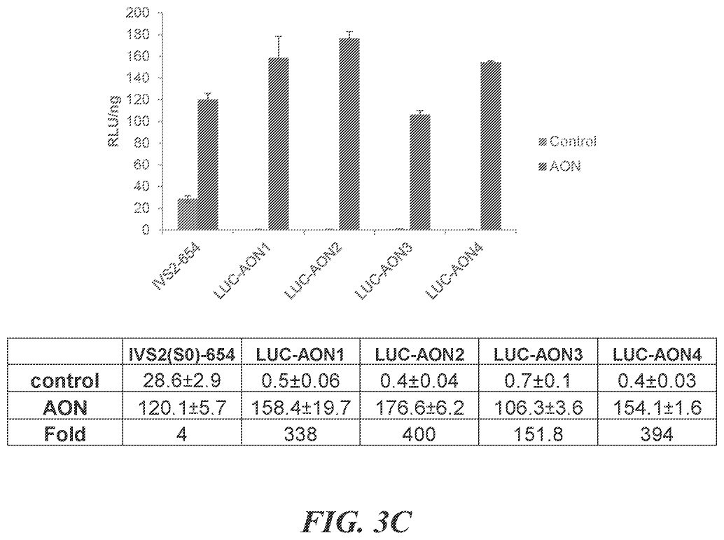

Four constructs that contain different sequences at the flanking region of the 5′ alternative splice site IVS (S0)-654 were generated (FIG. 3A). 8 nucleotides of 5′ the alternative splice site, 651-658, were maintained which are critical for splicing, and mutated nucleotides outside of the splice site to have at least 5 nt differences from each other. The expression of each construct was tested in HEK293 cells to determine whether its transgene is induced by its corresponding AON, and is affected by other non-corresponding AONs. The induction of expression of the reporter gene was observed by the corresponding AON but not cross-modulation by other AONs (FIG. 3B). Even though induction efficiency is variable among the constructs, all four constructs resulted in improved levels of transgene induction compared to IVS (S0)-654 (FIG. 3C). These data confirmed that the splicing of the transgene is controlled in a highly sequence-specific manner by the AON, allowing for the differential regulation of multiple transgenes.

Differential Regulation of Multiple Gene Expression by their Corresponding AON

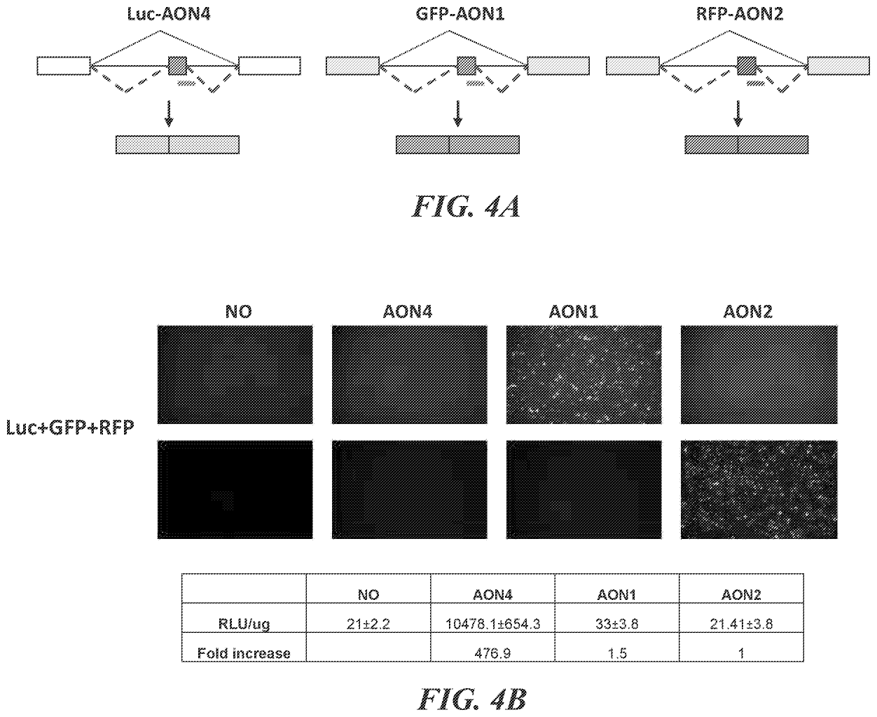

Differential expression of three different reporter genes with their corresponding AONs was tested. Modified intron AON4 was introduced into luciferase, AON1 into Green fluorescent protein (GFP), and AON2 into red fluorescent protein (RFP). Those reporter genes were subcloned into CBh backbone vector, individually (Luc-AON4, GFP-AON1, and RFP-AON2) (FIGS. 4A and 4B). The mixture of three plasmids was transfected into HEK293 cells, and the cells treated with individual AON, LNAAON4, LNAAON1, and LNAAON2, the day after transfection. It was observed that each AON induced its corresponding target gene specifically (FIG. 4B). These data indicated that the expression of multiple transgene can be regulated individually using the inducible vectors described herein and their corresponding AON.

Regulation of Luciferase Expression of AAV Vector that Carry Optimized IVS2 Mutant Intron by AON in Mouse Liver.

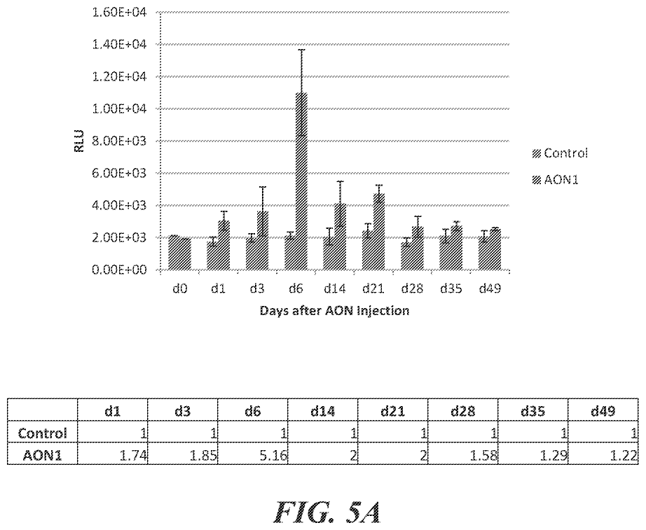

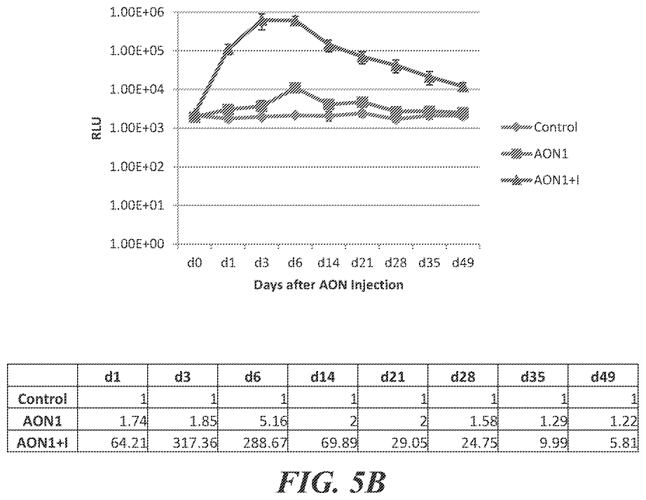

To demonstrate that the regulation system containing optimized small intron also can function to control transgene expression in animals, AAV2.5-CBh-Luc-AON1 vector was tested in 6-week-old female Balb/c mice. AAV vectors were injected into the mice retroorbitally at doses of 1×10¹¹ vg. At 6 weeks post-injection, mice were injected with LNAAON1 for two consecutive days and imaged for induction of luciferase expression. When the AAV was targeted to the liver, luciferase expression in the liver was induced by LNAAON1 administration for up to 5.2-fold increase (FIG. 5A). The luciferase expression peaked at day 6 and lasted 14 days. Results described herein showed that the optimized inducible system also could be used to control transgene expression in vivo. However, the induction level after AON administration was not great compared to in vitro data. One possible reason might be an inefficient delivery of AON to the target. To test this hypothesis, LNAAON1 was administered with cationic transfection reagent in vivo. With this reagent, luciferase expression in the liver was induced by LNAAON1 administration up to 317.4-fold and peaked at day 3 and gradually decreased, but lasted more than 45 days (FIG. 5B). These data indicated that delivery of AON to the target is one of the limiting factors in this system, and AON delivery to the target was improved dramatically.

Luciferase Expression of AAV2.5-CBh-Luc-DGT1 is Re-Inducible by Re-Administration of AON in Mouse Eyes.

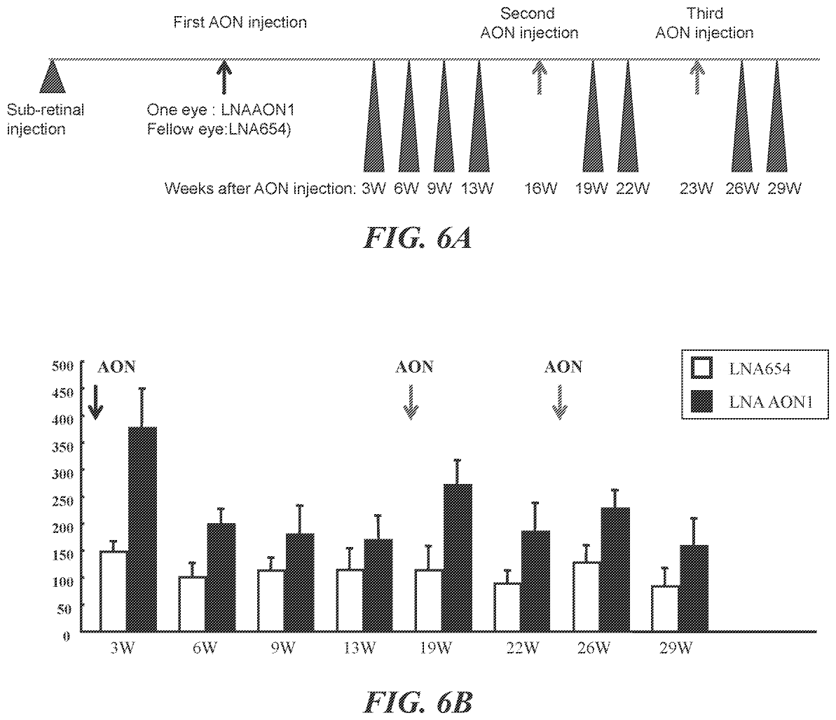

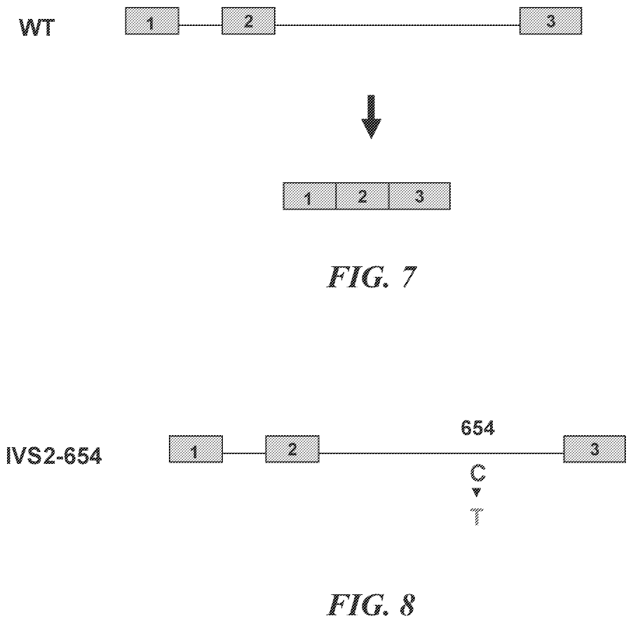

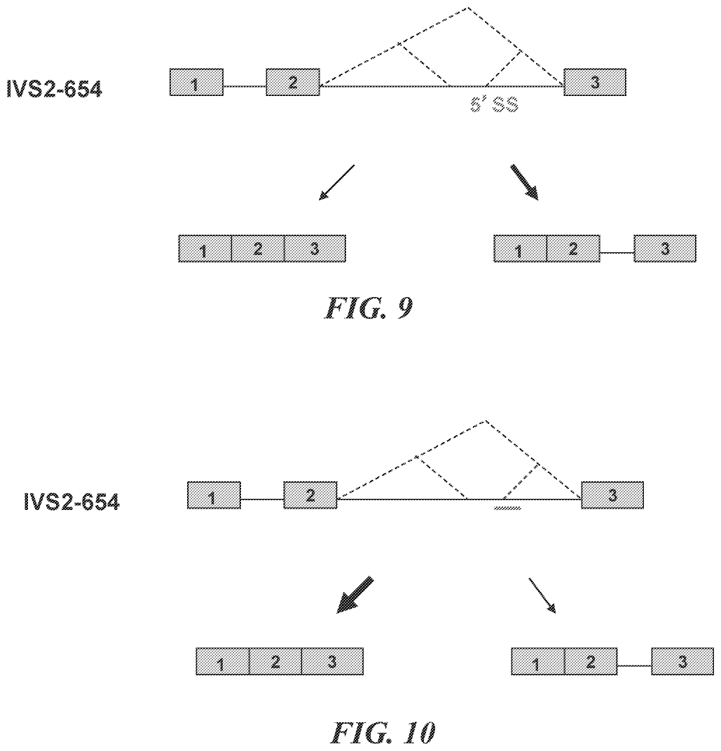

We tested inducible vector, Luc-AON1 under a promoter CBh using a modified AAV2 capsid, AAV2.5 in mouse eyes. Four weeks after subretinal injection of the viral vector, an intravitreal injection of the corresponding AON, LNAAON1, or mismatched AON, LNA654, was given. Three weeks after AON injection, mean luciferase activity was 2.5-fold higher in eyes injected with LNAAON1 than those injected with LNA654 (P=0.0038, FIG. 6). Mean luciferase activity was reduced at 6 and 9 weeks after injection of LNAAON1, but still significantly greater than that in eyes injected with LNA654. At 13 weeks after AON injection there was no longer a statistically significant difference, therefore at 16 weeks a second intravitreal injection of AON was given. Three weeks later, mean luciferase activity had increased in LNAAON1-injected eyes and was 2-fold higher than that in LNA654-injected eyes (P=0.017). Three weeks later the difference in luciferase activity was no longer significant (P=0.079). A third intravitreal injection of AON was done at week 23. Three weeks later there was no statistically significant difference in luciferase activity between LNAAON1-injected and LNA654-injected eyes. These data provide proof-of-concept for use of the inducible system in the eye and show that at least one re-induction is possible, but the magnitude of induction may degrade over time.

### Discussion

The study presented herein successfully demonstrated improvement of induction of luciferase expression in vitro mediated by an optimized inducible vector, AAV2.5-CBh-Luc-AON1. Induction of luciferase expression in mouse liver and eye with the same vector was also successfully demonstrated. Modification of nucleotide T and A to G and T at IVS2 intron 657 and 658 increased induction of luciferase more than 100-fold by AON, compared to without AON, by reducing background expression significantly. It is likely due to tight regulation of the splicing process by increasing the strength of the alternatively used 5′ splice site by making that splice site more close to consensus. Generation of small IVS2-654 intron, S0, 247 nt in length, without change in induction strength compared to original IVS2-654, 850 nt in length, allowed more cloning capacity for transgene in AAV system. Together, the optimized inducible system could be useful for controlling transgene expression mediated by AAV.

Angiogenesis is a complex multi-step process that involves the sprouting of vascular endothelial cells from existing vessels through endothelial cell proliferation, migration, tube formation and remodeling of extracellular matrix. This process is controlled by complex interactions between growth factors, extracellular matrix and cellular components, the net outcome being determined by the balance of angiogenic and angiostatic elements. A number of growth factor molecules are involved in the control of angiogenesis, and the therapeutic manipulation of one or a combination of these offers the potential means to control neovascularization in the eye. To date, cytokines that have been targeted and/or angiostatic proteins that have been bolstered using a gene therapy approach in experimental models include vascular endothelial growth factor (VEGF), insulin-like growth factor-1 (IGF-1), pigment epithelium-derived factor (PEDF), matrix metalloproteinases (MMPs), angiostatin, endostatin and integrins. However, none have achieved near complete regression of neovascularization. The effective control of angiogenesis in patients with retinal neovascular disorders is likely to require the long-term presence of angiostatic protein in the eye. Inappropriate inhibition of neovascularization could cause damage to normal ocular structures. Therefore, development of strategies to enable appropriate regulation of gene expression is desirable to minimize the potential for local toxicity. In the current study, it was successfully demonstrated that the expression of transgene using the optimized inducible system can be controlled in mouse eye. In mouse eyes, specific induction of luciferase activity was demonstrated by AON administration after transduction with AAV2.5 vectors that carry DGT1 intron containing the luciferase gene. It was also demonstrated that the system is re-inducible by re-administration of AON in mouse eyes. Moreover, individual expression of three different reporter genes with their corresponding AON was successfully demonstrated. AON4, AON1, and AON2 independently regulated, without any crossover, the expression of luciferase, GFP and RFP, respectively. 16-mer AON that is complementary to the alternatively used 5′ splice site and its flanking sequences to each target transgene was used to individually induce the expression. This 16-nucleotide region is composed of 8 nucleotides that are essential for splice site, and 8 nucleotides for flanking region. There are 8 bases in the flanking sequences that could be mutated without affecting the strength of the alternative splice site. It was shown that each AON has 6-7 mismatches with each other, and did not cross-modulate the alternative splicing of target genes. Therefore, within the target region of the 5′ splice site, there are more bases than needed (8>6) that could be mutated to create different target sequences that would not be cross modulated by other AON. Such a capacity of transgene regulation would be impossible for the commonly used regulation systems such as the tet-on and the rapamycin inducible systems. In fact, each of these systems can independently regulate only one transgene in theory. Altogether, these data indicated that the novel optimized regulation system could be a very useful strategy to apply clinically to differentially regulate the expression of multiple transgenes for gene therapy of clinically relevant diseases like ocular neovascularization.

### Materials and Methods

Maintenance of cells. Human embryonic kidney (HEK) 293 cells were maintained in Dulbecco's modified Eagle's medium with 10% heat-inactivated fetal bovine serum and 1× Penn/Strep (DMEM+, Sigma). Cells were grown at 37° C. in a 5% CO₂ humidified incubator.

AAV vector plasmids. All AAV vector plasmids carrying Luciferase were generated from pTR-CBh-LuciferaseGL3+NotI (Xiaohuai et al). The Intron region was subcloned into this plasmid using SphI and XcmI restriction enzyme digestion. Mutations at the alternatively used 5′ splice site of IVS2-654 were made using standard PCR techniques, and were sequenced to ensure that they were as expected.

pZsGreen 1-Dr (#632428) and pDsRed-Express-Dr (#632423) were purchased from Clontech. The luciferase coding region was removed using AgeI and NotI from pTR-CBh-Luciferase GL3+NotI plasmid and replaced with ZsGreen1-Dr or DsRed-Express-Dr coding region, and named pTR-CBh-ZsGreen1-Dr, and pTR-CBh-DsRed-Express-Dr, respectively. Then, mutated IVS (S0)-654 intron, AON1 was inserted into the ZsGreen1-Dr coding region of pTR-CBh-ZsGreen1-Dr, and named pTR-CBh-ZsGreen1-Dr-AON1. Modified IVS (S0)-654 intron, AON2 was also inserted into the DsRed-Express-Dr coding region of pTR-CBh-DsRed-Express-Dr, and named pTR-CBh-RedDr-AON2.

Antisense oligonucleotides. Modified antisense oligonucleotides, LNAs, were purchased from Exiqon. LNA-DGT1 was generously provided by Dr. Juliano at UNC. In Table 4, capital letters denote LNA base, and lower case letters denote nature DNA bases.

AAV vector production and characterization. Recombinant AAV vectors were generated using HEK293 cells grown in serum-free suspension conditions in shaker flasks as described in Grieger et al. (manuscript in preparation). In brief, the suspension HEK293 cells were transfected using polyethyleneimine (Polysciences) and the following plasmids: pXX680, pXR2.5, and pTR-CBh-Luc-AON1 to generate AAV carrying CBh-Luc-AON1. 48 hours post-transfection, cell cultures were centrifuged and supernatant was discarded. The cells were resuspended and lysed through sonication. 550 U of DNase was added to the lysate and incubated at 37° C. for 45 minutes, followed by centrifugation at 9400×g to pellet the cell debris and the clarified lysate was loaded onto a modified discontinuous Iodixanol gradient followed by column chromatography. The physical particle titer of each AAV vector preparation was then determined using a QPCR assay as described previously.

Characterization of transgene expression in vitro. Three marker genes, firefly luciferase, ZsGreen1-Dr, and DsRed-Express-Dr, were used for studying the regulation of transgene expression in vitro using cultured cell lines in 24-well plates. For measuring Luciferase activity, cells in each 24-well plate were transfected with 500 ng of the corresponding plasmid and 10 pmole of AON as indicated using the PEI transfection method. At 24 hours after transfection, the cells were lysed with 100 μl of 1× Reporter Lysis Buffer (Promega, cat #E4030). 20 ul of the lysate was then mixed with 100 μl of luciferase substrate (Promega, cat #E4030) to determine the luciferase activity.

For studies involving the ZsGreen1-Dr, and DsRed-Express-Dr marker gene, cells were transfected with 500 ng of plasmids with 10 pmole of AON using the PEI transfection method. After transfection, the cells were cultured for another 48 hours and imaged using fluorescent microscopy.

Characterization of transgene expression in vivo. Luciferase was used for studying the regulation of transgene expression in 6-week-old female Balb/c mice. AAV vectors, AAV2.5-CBh-Luc-WT and AAV2.5-CBh-Luc-AON1 were targeted to the liver via retro orbital injection at doses of 1×10¹¹ vg. At 6 weeks after virus injection, the animals were imaged for basal level of luciferase transgene expression using the following procedures: Mice were anesthetized by Isoflulane. Luciferin (125 μl at 25 mg/ml) was then injected i.p. into each mouse to allow the in vivo assay of luciferase activity. The mice were then imaged using the IVIS imaging system (Xenogen). To turn on the expression of the luciferase transgene, AON or AON with invivofectamine at 25 mg/kg were injected retro orbitally for two consecutive days. The mice were then imaged as describe above at days indicated starting from the last day of AON injection.

For testing inducible AAV vectors in eyes, mice were treated humanely in strict compliance with the Association for Research in Vision and Ophthalmology statement on the use of animals in research. Four-week-old Balb/c mice were given a subretinal injection of 1 μl containing 10⁹ genome particles of AAV2.5-CBh-Luc-AON1 or AAV2.5-CBh-Luc-WT with a Harvard pump apparatus and pulled glass micropipettes as previously described (Mori et al.). Four weeks after injection of vector, mice were given an intravitreal injection of 1 μl containing 0.556 μg of LNAAON1 or LNA654. The mice were then imaged as describe above at days indicated starting from the last day of AON injection.

### REFERENCE

1. Mori K, Duh E, Gehlbach P, Ando A, Takahashi K, Pearlman J, Mori K, Yang H S, Zack D J, Ettyreddy D, Brough D E, Wei L L, Campochiaro P A: Pigment epithelium-derived factor inhibits retinal and choroidal neovascularization. J Cell. Physiol. 188:253-263, 2001

### Example 2. Generation of saCa9 Comprising Regulatory Nucleic Acid Sequence

saCas9 comprising the regulatory sequence (beta-globin intron region) is generated as described in Example 1. The regulatory sequence intron region (e.g., SEQ ID NO:53 (IVS2-654 intron with 200 by deletion) is subcloned into an AAV vector plasmid carrying saCas9 using restriction digestion.

### Example 3. Measuring Off Target Effects of Gene Editing

Digested genome sequencing (Digenome-seq), is an in vitro Cas9-digested whole-genome sequencing, that is a robust, sensitive, unbiased, and cost-effective method for profiling genome-wide off-target effects of programmable nucleases, for example Cas9, in mammalian, e.g., human, cells.

HeLa, HEK, and CHO cells expressing a Nav 1.8-directed gRNA are transfected with (1) no nuclease (e.g., a untransfected population); (2) a constitutively active Casp9; (3) the gene editing system described herein without the oligonucleotide that binds the regulatory sequence, e.g., a nuclease in the “OFF” position; and (4) the gene editing system described herein and the oligonucleotide that binds the regulatory sequence, e.g., a nuclease in the “ON” position using lipofectamine 2000 (Life Technologies). HeLa cells are cultured in DMEM medium containing 10% FBS. Cells are incubated for 48 hours.

In Vitro Cleavage of Genomic DNA.

Then, using DNeasy Tissue kit (Qiagen), intact genomic DNA is isolated from each cell population. DNA isolated from the untransfected cell population is incubated with and without the constitutively active nuclease described herein, independently, to allow for digestion of the isolated DNA. DNA isolated from the nuclease-expressing populations are isolated with their indicated nuclease to allow for digestion of the isolated DNA. This reaction is carried out at 37° C. in a reaction buffer (100 mM NaCl, 50 mM Tris-HCl, 10 mM MgCl₂, and 100 μg/ml BSA) for 8 hours. At the end of the reaction, RNase A (50 μg/mL) is added to degrade the sgRNA. Digested DNA is purified by DNeasy Tissue kit (Qiagen).

Full Genome Sequencing and Digenome-Seq.

Purified digested DNA is analyzed via whole genome sequencing using standard methods. Digestion with the nuclease produces DNA fragments with identical 5′ ends, which give rise to sequence reads that are vertically aligned at cleavage sites. In contrast, all other sequence reads without identical 5′ ends would be aligned in a staggered manner. Sequence reads are mapped to the reference genome, and the Integrative Genomics Viewer (IGV) is used to observe patterns of sequence alignments at the on-target (e.g., Nav 1.8 sequence) and the off-target sites (e.g., non-Nav 1.8 sequence). IGV is available on the world wide web at, e.g., softward.broadinstitute.org/software/igv/. Digenome-Seq is further described in, for example, international Patent App. No. WO 2016/0766721; Kim, et al. Nat Methods, 2015, 12: 237-243; Mei et al. J Genet Genomics. 2016; 43:63-75; Hu, et al. Nat Protoc. 2016; 11: 853-871; each of which are incorporated herein by reference in their entireties. Additional programs to analyze Digenome-seq data are available on the world wide web, for example, at rgenome.net/digenome/portable.

Off-target effects for the constitutively active Cas9 are compared to any off targets effects observed in the untransfected cell population digested with constitutively active Cas9. Common off-target sites are identified and removed from consideration, as are any common off-target sites identified between the nuclease-digested and no nuclease-digested untransfected cell populations. Off-target sites identified in the “ON” nuclease population are compared to the “OFF” nuclease population and removed from consideration. These sites removed from consideration, e.g., to be identified as a true off-target effect, are done so as they are unlikely to be caused by off target editing by the nuclease.

Digenome-seq reveal that in HeLa cells constitutively active Cas9 results in an increased incidence of off-target effects, e.g., editing, as compared to the “ON” gene editing system described herein, indicating that the gene editing system described herein provides a markedly reduced rate of off target effects as compared to conventional CRISPR/Cas9 gene editing. Moreover, off-target editing and on-target editing, e.g., reveal editing at the Nav 1.8 sequence does not occur in cells expressing the “OFF” gene editing system indicating that the gene editing system described herein provides temporal and spatial control of gene editing. Further, these results were recapitulated in all cell types tested herein, indicating that reduced off-target effects is a feature to the gene editing system, and not cell-type specific.

1. A system for editing a gene (e.g., altering expression of at least one gene product) having reduced off target effects comprising introducing into a cell having a target gene sequence
a) a vector comprising a nucleic acid sequence encoding a nuclease, wherein the nucleic acid encoding the nuclease contains within its sequence a regulatory nucleic acid sequence having a first and second set of splice elements defining a first and second intron, wherein the first and second intron flank a sequence encoding a non-naturally occurring exon sequence containing an in-frame stop codon sequence, and wherein the first and second intron are spliced from the pre-mRNA message to produce an mRNA encoding a non-functional nuclease that contains an amino acid sequence encoded by the non-naturally occurring exon; and
b) an oligonucleotide that binds to the regulatory nucleic acid sequence, wherein within the cell the oligonucleotide prevents splicing of the second set of splice elements from the mRNA, thereby producing an mRNA that lacks the exon and encodes a nuclease that is functional for gene editing of a target gene.

2. The system of claim 1, wherein the nuclease is selected from the group consisting of a CRISPR-associated nuclease, a meganuclease, a zinc finger nuclease, and a transcription activator-like effector nuclease.

3. The system of claim 1, wherein the nuclease is an endonuclease or an exonuclease.

4. The system of claim 1, wherein component (a) further comprises a gRNA that binds to the sequence of the target gene.

5. The system of claim 1, wherein the regulatory nucleic acid sequence is a beta-globin mutant intron.

6. (canceled)

7. The system of claim 1, wherein the regulatory nucleic acid sequence comprises a sequence selected from the group consisting of: SEQ ID NO: 18 (IVS2-654 intron C-T), SEQ ID NO:50 (IVS2-654 intron with 564CT mutation), SEQ ID NO:51 (IVS2-654 intron with 657G mutation), SEQ ID NO:52 (IVS2-654 intron with 658T mutation), SEQ ID NO:20 (IVS2-654 intron with 657GT mutation), SEQ ID NO:53 (IVS2-654 intron with 200 by deletion), SEQ ID NO:68 (IVS2-654 intron with only 197 bp), SEQ ID NO:55 (IVS2-654 intron with 6A mutation), SEQ ID NO:56 (IVS2-654 intron with 564C mutation), SEQ ID NO:57 (IVS2-654 intron with 841A mutation), SEQ ID NO:59 (IVS2-705 intron with 564CT mutation), SEQ ID NO:60 (IVS2-705 intron with 657G mutation), SEQ ID NO:61 (IVS2-705 intron with 658T mutation), SEQ ID NO:62 (IVS2-705 intron with 657GT mutation), SEQ ID NO:63 (IVS2-705 intron with 200 by deletion), SEQ ID NO:64 (IVS2-705 intron with 425 by deletion), SEQ ID NO:65 (IVS2-705 intron with 6A mutation), SEQ ID NO:66 (IVS2-705 intron with 564C mutation), SEQ ID NO:67 (IVS2-705 intron with 841A mutation), SEQ ID NO: 74, SEQ ID NO:75, SEQ ID NO; 76, SEQ ID NO: 77, SEQ ID NO:78, SEQ ID NO: 143, SEQ ID NO: 144, SEQ ID NO: 145, SEQ ID NO: 146, SEQ ID NO: 147, SEQ ID NO: 148; and in any combination thereof, including singly.

8. The system of claim 1, wherein the oligonucleotide that binds to the regulatory sequence comprises a sequence selected from the group consisting of: SEQ ID NO:37 (oligo for IVS2-654 CT), SEQ ID NO:38 (oligo for IVS2-654 with 657GT mutation), SEQ ID NO:39 (oligo for 6A mutation in IVS2-654), SEQ ID NO:40 (oligo for 564C mutation in IVS2-654), SEQ ID NO:41 (oligo for 564CT mutation in IVS2-654), SEQ ID NO:43 (oligo for 841A mutation in IVS2-654), SEQ ID NO:44 (oligo for 657G mutation in IVS2-654), SEQ ID NO:45 (oligo for 658T mutation in IVS2-654), SEQ ID NO:42 (oligo for 705G mutation in IVS2-705), SEQ ID NO:49 (oligo for IVS2-705), SEQ ID NO:76 (Antisense exon 23 skipping inducing oligo) respectively, and SEQ ID NO 138 (Oligo for LUC-AON1), SEQ ID NO: 139 (oligo for LUC-AON2), SEQ ID NO: 140 (Oligo for LUC-AON3), SEQ ID NO: 141 (Oligo for LUC-AON4), SEQ ID NO: 142 (Oligo for IVS2(S0)-654, LUC-654) and SEQ ID NO: 149 (Oligo for WT regulatory).

9. (canceled)

10. The system of claim 1, wherein the off-target effects are reduced by at least 30%, at least 40%, at least 50%, at least 60%, at least 70%, at least 80%, or at least 90% or more.

11. The system of claim 1, wherein components (a) and (b) are located on same or different vectors.

12. The system of claim 1, wherein component (b) is introduced to cell as naked DNA, as a lipid formulation, or as a nanoparticle.

13. (canceled)

14. (canceled)

15. The system of claim 1, wherein component (b) is administered at a time point following the administration of (a), or components (a) and (b) are administered at substantially the same time.

16. (canceled)

17. The system of claim 1, wherein the expression of (a) is not detected in the cell in the absence of (b), or absence of expression of (b).

18. (canceled)

19. The system of claim 1, wherein component (b) controls an “ON” and/or “OFF” status of the system.

20. (canceled)

21. (canceled)

22. The system of claim 1, wherein the vector is a viral vector or a non-viral vector.

23. The system of claim 22, wherein the viral vector is selected form the group consisting of: from the group consisting of an AAV vector, an adenovirus vector, a lentivirus vector, a retrovirus vector, a herpesvirus vector, an alphavirus vector, a poxvirus vector, a baculovirus vector and a chimeric virus vector.

24. (canceled)

25. (canceled)

26. The system of claim 2, wherein the CRISPR-associated nuclease
a) creates double stand breaks for gene editing and wherein the CRISPR-associated nuclease is selected from the group consisting of Cpf1, C2c1, C2c3, Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, C2c1, C2c3, Cas12a, Cas12b, Cas12c, Cas12d, Cas12e, Cas13a, Cas13b, and Cas13c;
b) is a Cas9 variant selected from Staphylococcus aureus (SaCas9), Streptococcus thermophilus (StCas9), Neisseria meningitidis (NmCas9), Francisella novicida (FnCas9), and Campylobacter jejuni (Cj Cas9); or
c) has been modified for gene-editing without double strand DNA breaks (such as CRISPRi or CRISPRa) and is selected from the group consisting of dCas, nCas, and Cas 13.

27. (canceled)

28. (canceled)

29. The system of claim 2, wherein the CRISPR-associated nuclease is codon optimized for expression in the eukaryotic cell.

30. The system of claim 1, wherein the gene editing is decreasing or increasing the expression of one or more gene products.

31. (canceled)

32. (canceled)

33. The system of claim 1, wherein the cell is in vivo or in vitro.

34. (canceled)

35. (canceled)

36. A method for editing a gene in a subject, the method comprising administering the system of claim 1 to a subject in need of gene editing.


SEQUENCE LISTING (154 sequences)

SEQ ID NO: 1 - DNA, 7713 bp, Artificial
  Plasmid TRCBA-int-luc mut (654 C-T)
  Intron (2739)..(3588)
gggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttcaatattggccattagccatattattcattggttatatagcataaatcaatattggatattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtatgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttactttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcgctattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatactttcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgcggataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgct

SEQ ID NO: 2 - DNA, 7713 bp, Artificial
  Plasmid TRCBA-int-luc (wt)
  Intron (2739)..(3588)
gggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttcaatattggccattagccatattattcattggttatatagcataaatcaatattggatattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtatgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttactttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcgctattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatactttcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgcggataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgct

SEQ ID NO: 3 - DNA, 7713 bp, Artificial
  Plasmid TRCBA-int-luc (654 C-T, 657 TA-GT)
  Intron (2739)..(3588)
gggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttcaatattggccattagccatattattcattggttatatagcataaatcaatattggatattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtatgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttactttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaagtgcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcgctattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatactttcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgcggataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgct

SEQ ID NO: 4 - DNA, 5860 bp, Artificial
  Plasmid GL3-int-Luc mut (654 C-T)
  Intron (948)..(1797)
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 5 - DNA, 5860 bp, Artificial
  Plasmid GL3-int-Luc (wt)
  Intron (948)..(1797)
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 6 - DNA, 5860 bp, Artificial
  Plasmid GL3-int-Luc (654 C-T, 657 TA-GT)
  Intron (48)..(1797)
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaagtgcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 7 - DNA, 6683 bp, Artificial
  Plasmid GL3-2int-fron-sph (mut)
  Intron (251)..(1100)
  Intron (1771)..(2620)
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttgtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 8 - DNA, 7547 bp, Artificial
  Plasmid GL3-3int-2fron-sph (mut)
  Intron (251)..(1100)
  Intron (1111)..(1960)
  Intron (2635)..(3484)
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttgtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagccatgagcttgtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagccatgcatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 9 - DNA, 5860 bp, Artificial
  Plasmid GL3-int-luc A (mut)
  Intron (673)..(1522)
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 10 - DNA, 5860 bp, Artificial
  Plasmid GL3-int-Luc B
  Intron (1440)..(2289)
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 11 - DNA, 5860 bp, Artificial
  Plasmid GL3-int-Luc C
  Intron (1691)..(2540)
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 12 - DNA, 5833 bp, Artificial
  Plasmid GL3-int-fron (mut)
  Intron (251)..(1100)
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttgtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 13 - DNA, 6710 bp, Artificial
  Plasmid GL3-2int-sph (mut)
  Intron (948)..(1797)
  Intron (1798)..(2647)
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 14 - DNA, 6710 bp, Artificial
  Plasmid GL3-2int-Sph-C
  Intron (948)..(1797)
  Intron (2541)..(3390)
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 15 - DNA, 5660 bp, Artificial
  Plasmid GL3-sint200-sph (mut)
  Intron (948)..(1597)
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagttgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 16 - DNA, 5660 bp, Artificial
  Plasmid GL3-sint200-sph (657 GT)
  Intron (948)..(1597)
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagttgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaagtgcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 17 - DNA, 5436 bp, Artificial
  Plasmid GL3-sint425-sph
  Intron (948)..(1373)
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttcctgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 18 - DNA, 850 bp, Artificial
  mutant intron (654 C-T)
  misc_feature (654)..(654): beta-globin intron 654 C-T mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 19 - DNA, 850 bp, Homo sapiens
  misc_feature (1)..(850): Wild-type beta-globin intron
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 20 - DNA, 850 bp, Artificial
  intron with two mutations (654 C-T; 657 TA-GT)
  misc_feature (654)..(654): beta-globin intron 654 C-T mutation
  misc_feature (657)..(658): beta-globin intron 657 TA-GT mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaagtgcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 21 - DNA, 2503 bp, Artificial
  luciferase cDNA with mutant intron (654 C-T)
  Intron (669)..(1518)
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 22 - DNA, 2503 bp, Artificial
  luciferase cDNA with wild type intron
  Intron (669)..(1518)
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 23 - DNA, 2503 bp, Artificial
  luciferase cDNA with double mutant intron (C654 C-T; 657 TA-GT)
  Intron (669)..(1518)
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaagtgcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 24 - DNA, 3355 bp, Artificial
  luciferase cDNA with mutant intron (654 C-T)
  Intron (1)..(850)
  Intron (1521)..(2370)
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 25 - DNA, 4219 bp, Artificial
  luciferase cDNA with mutant intron (654 C-T)
  Intron (1)..(850)
  Intron (861)..(1710)
  Intron (2385)..(3234)
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagccatgagcttgtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagccatgcatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 26 - DNA, 2503 bp, Artificial
  luciferase cDNA with mutant intron (654 C-T) at alternative location A
  Intron (394)..(1243)
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 27 - DNA, 2503 bp, Artificial
  luciferase cDNA with mutant intron (654 C-T) at alternative location B
  Intron (1161)..(2010)
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 28 - DNA, 2503 bp, Artificial
  luciferase cDNA with mutant intron (654 C-T) at alternative location C
  Intron (1412)..(2261)
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 29 - DNA, 2505 bp, Artificial
  luciferase cDNA with mutant intron (654 C-T) upstream of translation site
  Intron (1)..(850)
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 30 - DNA, 3353 bp, Artificial
  luciferase cDNA with two mutant introns (654 C-T)
  Intron (669)..(1518)
  Intron (1519)..(2368)
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 31 - DNA, 3353 bp, Artificial
  luciferase cDNA with two mutant introns (654 C-T)
  Intron (669)..(1518)
  Intron (2262)..(3111)
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 32 - DNA, 2303 bp, Artificial
  luciferase cDNA with mutant intron (654 C-T)
  Intron (669)..(1318)
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagttgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 33 - DNA, 2303 bp, Artificial
  luciferase cDNA with double mutant intron (654 C-T; 657 TA-GT)
  Intron (669)..(1318)
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagttgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaagtgcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 34 - DNA, 2079 bp, Artificial
  luciferase cDNA with mutant intron (654 C-T)
  Intron (669)..(1094)
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttcctgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 35 - DNA, 7449 bp, Artificial
  plasmid TRCBA with alpha antitrypsin cDNA and mutant intron (654 C-T)
  Intron (2866)..(3715): Mutant beta-globin intron (654C-T)
gggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttcaatattggccattagccatattattcattggttatatagcataaatcaatattggatattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtatgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttactttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgatatcaagcttggggattttcaggcaccaccactgacctgggacagtgaatcgacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagagccatgaaggctgccaggaactcctccgtaccctcaaccagccagacagccagctccagctgaccaccggcaatggcctgtgcctcagcgagggcctgaagcaagtggataagtttttggaggatgttaaaaagttgtaccactcataagccttcactgtcaacttcggggacaccgaagaggccaagaaacagatcaacgattacgttgagaagggtactcaagggaaaatggtggatgtggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttagtcatgtttaacatccagcactgtaaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagaagctttccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccgtggtcacagaggaggcacccctgaagctctccaatgccgtgcataaggctgtgctgaccatcgacgagaaagggactgaagctgctggggccatgtttttagaggccatacccatgtctatcccccccgaggtcaaggtcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctggtaaccccccccccccctgcaggggccctcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcgctattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatactttcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgcggataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgct

SEQ ID NO: 36 - DNA, 2107 bp, Artificial
  alpha antitrypsin cDNA with mutant intron (654 C-T)
  Intron (772)..(1621): Mutant beta globin intron (654C-T)
atgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagagccatgaaggctgccaggaactcctccgtaccctcaaccagccagacagccagctccagctgaccaccggcaatggcctgtgcctcagcgagggcctgaagcaagtggataagtttttggaggatgttaaaaagttgtaccactcataagccttcactgtcaacttcggggacaccgaagaggccaagaaacagatcaacgattacgttgagaagggtactcaagggaaaatggtggatgtggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttagtcatgtttaacatccagcactgtaaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagaagctttccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccgtggtcacagaggaggcacccctgaagctctccaatgccgtgcataaggctgtgctgaccatcgacgagaaagggactgaagctgctggggccatgtttttagaggccatacccatgtctatcccccccgaggtcaaggtcaacaaaccctttgtcttcttaatgattgaacaaaataccaagtctcccctcttcatgggaaaagtggtgaatcccacccaaaaataa

SEQ ID NO: 37 - DNA, 18 bp, Artificial
  regulatory sequence-binding oligonucleotide
gctattaccttaacccag

SEQ ID NO: 38 - DNA, 18 bp, Artificial
  regulatory sequence-binding oligonucleotide
gcacttaccttaacccag

SEQ ID NO: 39 - DNA, 18 bp, Artificial
  oligo for 6A mutation in IVS2-654
caagggtcccatagtctc

SEQ ID NO: 40 - DNA, 18 bp, Artificial
  oligo for 564C mutation in IVS2-654
gaaagagatgagggaaag

SEQ ID NO: 41 - DNA, 18 bp, Artificial
  oligo for 564CT mutation in IVS2-654
gaaagagaagagggaaag

SEQ ID NO: 42 - DNA, 18 bp, Artificial
  oligo for 705G mutation in IVS2-705
cctcttacctcagttaca

SEQ ID NO: 43 - DNA, 18 bp, Artificial
  oligo for 841A mutation in IVS2-654
ctgtgggagtaagataag

SEQ ID NO: 44 - DNA, 18 bp, Artificial
  oligo for 657G mutation in IVS2-654
gctcttaccttaacccag

SEQ ID NO: 45 - DNA, 18 bp, Artificial
  oligo for 658T mutation in IVS2-654
gcaattaccttaacccag

SEQ ID NO: 46 - DNA, 18 bp, Artificial
  oligo for IVS2-654
caagggtcccatagactc

SEQ ID NO: 47 - DNA, 18 bp, Artificial
  oligo for IVS2-654
gaaagagattagggaaag

SEQ ID NO: 48 - DNA, 18 bp, Artificial
  oligo for IVS2-654
ctgtgggaggaagataag

SEQ ID NO: 49 - DNA, 18 bp, Artificial
  oligo for IVS2-705
cctcttacatcagttaca

SEQ ID NO: 50 - DNA, 850 bp, Artificial
  IVS2-654 intron with 564CT mutation
  misc_feature (564)..(565): 564CT mutation
  misc_feature (654)..(654): 654T mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 51 - DNA, 850 bp, Artificial
  IVS2-654 intron with 657G mutation
  misc_feature (654)..(654): 654T mutation
  misc_feature (657)..(657): 657G mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaagagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 52 - DNA, 850 bp, Artificial
  IVS2-654 intron with 658T mutation
  misc_feature (654)..(654): 654T mutation
  misc_feature (658)..(658): 658T mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaattgcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 53 - DNA, 650 bp, Artificial
  IVS2-654 intron with 200 bp deletion
  misc_feature (454)..(454): C to T mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagttgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 54 - DNA, 426 bp, Artificial
  IVS2-654 intron with 425 bp deletion
  misc_feature (230)..(230): C to T mutation
gtgagtctatgggacccttgatgttttctttcctgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 55 - DNA, 850 bp, Artificial
  IVS2-654 intron with 6A mutation
  misc_feature (6)..(6): 6A mutation
  misc_feature (654)..(654): 654T mutation
gtgagactatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 56 - DNA, 850 bp, Artificial
  IVS2-654 intron with 564C mutation
  misc_feature (564)..(564): 564C mutation
  misc_feature (654)..(654): 654T mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 57 - DNA, 850 bp, Artificial
  IVS2-654 intron with 841A mutation
  misc_feature (654)..(654): 654T mutation
  misc_feature (841)..(841): 841A mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttactcccacag

SEQ ID NO: 58 - DNA, 850 bp, Artificial
  IVS2-705 intron
  misc_feature (705)..(705): 705G mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgaggtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 59 - DNA, 850 bp, Artificial
  IVS2-705 intron with 564 CT mutation
  misc_feature (564)..(565): 564CT mutation
  misc_feature (705)..(705): 705G mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttctgggttaaggcaatagcaatatttctgcatataaatatttctgcatataaattgtaactgaggtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 60 - DNA, 850 bp, Artificial
  IVS2-705 intron with 657G mutation
  misc_feature (657)..(657): 657G mutation
  misc_feature (705)..(705): 705G mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaagagcaatatttctgcatataaatatttctgcatataaattgtaactgaggtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 61 - DNA, 850 bp, Artificial
  IVS2-705 intron with 658T mutation
  misc_feature (658)..(658): 658T mutation
  misc_feature (705)..(705): 705G mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaattgcaatatttctgcatataaatatttctgcatataaattgtaactgaggtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 62 - DNA, 850 bp, Artificial
  IVS2-705 intron with 657GT mutation
  misc_feature (657)..(658): 657GT mutation
  misc_feature (705)..(705): 705G mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaagtgcaatatttctgcatataaatatttctgcatataaattgtaactgaggtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 63 - DNA, 650 bp, Artificial
  IVS2-705 intron with 200 bp deletion
  misc_feature (505)..(505): T to G mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagttgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgaggtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 64 - DNA, 426 bp, Artificial
  IVS2-705 intron with 425 bp deletion
  misc_feature (281)..(281): T to G mutation
gtgagtctatgggacccttgatgttttctttcctgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgaggtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 65 - DNA, 850 bp, Artificial
  IVS2-705 intron with 6A mutation
  misc_feature (6)..(6): 6A mutation
  misc_feature (705)..(705): 705G mutation
gtgagactatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgaggtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 66 - DNA, 850 bp, Artificial
  IVS2-705 intron with 564C mutation
  misc_feature (564)..(564): 564C mutation
  misc_feature (705)..(705): 705G mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcatctctttctttcagggcaataatgatacaatgtatcatgcctctttgcaccattctaaagaataacagtgataatttctgggttaaggcaatagcaatatttctgcatataaatatttctgcatataaattgtaactgaggtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 67 - DNA, 850 bp, Artificial
  IVS2-705 intron with 841A mutation
  misc_feature (705)..(705): 705G mutation
  misc_feature (841)..(841): 841A mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgaggtaagaggtttcatattgctaatagcagctacaatccagctaccattctgcttttattttatggttgggataaggctggattattctgagtccaagctaggcccttttgctaatcatgttcatacctcttatcttactcccacag

SEQ ID NO: 68 - DNA, 196 bp, Artificial
  IVS2-654 intron 197 bp
gtgagtctatgggacccttgatgttcttttaatatacttttttgtttatcttatttctaatactttccctcttctctttctttcaggtgattgactgactgggttaaggtaatagcgccgttgaaaacctcagccgtatagtccaagctaggcccttttgctaatcatgttcatacctcttatcttcctcccacag

SEQ ID NO: 69 - DNA, 247 bp, Artificial
  IVS-654 intron 247 bp
gtgagtctatgggacccttgatgttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttagtccaagctaggcccttttgctaatcatgttcatacctcttatcttcctcccacag

SEQ ID NO: 70 - DNA, 14667 bp, Homo sapiens
  misc_feature (1)..(14667): CFTR gene exon 19
  misc_feature (12191)..(12191): 3849 + 10 kb C-to-T mutation site
gtgagatttgaacactgcttgctttgttagactgtgttcagtaagtgaatcccagtagcctgaagcaatgtgttagcagaatctatttgtaacattattattgtacagtagaatcaatattaaacacacatgttttattatatggagtcattatttttaatatgaaatttaatttgcagagtcctgaacctatataatgggtttattttaaatgtgattgtacttgcagaatatctaattaattgctaggttaataactaaagaagccattaaataaatcaaaattgtaacatgttttagatttcccatcttgaaaatgtcttccaaaaatatcttattgctgactccatctattgtcttaaattttatctaagttccattctgccaaacaagtgatactttttttctagcttttttcagtttgtttgttttgtttttctttgaagttttaattcagacatagattattttttcccagttatttactatatttattaagcatgagtaattgacattattttgaaatccttcttatggatcccagcactgggctgaacacatagaaggaacttaatatatactgatttctggaattgattcttggagacagggatggtcattatccatatacttcaggctccataaacatatttcttaattgccttcaaatccctattctggactgctctataaatctagacaagagtattatatattttgattgatattttttagataaaataaaagggagctgaaaactgaattgcaaactgaattttaaaactttatctctctgtggttaattgcaaacacagatacaaaaatatagagagagatacagttagtaaagatgttaggtcaccgttactaacactgacatagaaacagttttgctcatgagtttcagaatatatgagtttgattttgcccatggattttagaatatttgataaacatttaatgcattgtacaaattctgtgaaaacatatatataggatgtgcgaaaagtccctgtgtatcatgtgaaatggcttaaaacagaacaccataggtattcatatcagtgaataccataggtagctgaaagtgttttttcctggggtcgccaagatgaatgccaaaagtgatatcattattataaacaatagccagaataggttggtataaacctggtagaaagccttgataaattgactttctctcctcctgacatcctgccacccctttgctttgctgatgctcatttgtccactaaattaaactcaagcaagccctagtaaagtaatagaatttgtggagtcctcattagtataggaagtttccctgatgtgagattagtaattagagatgtagcaaaatgagaaagaagtaatatgcttagatatttcattttctctgaacctgtatatacaaaataggccatgcgtgttcagtaactattcactgcaaggcactctctaggtactttgggggaattggaaattactcacataaggctatggattgtgccatttgtcaaaagacaaaatgacaacaaatttagtttaaagacctcagtcagctttattttctattctagatttggacagtccttcatttcacaaattggagtaagtgttccaataagttgagcaaaggagcttggctttatagacccaaaaaaagggccaaaggaagcagaaacaaagaacaataagagaattggtcatttcaaagttacttttcttgaaaggtggggacaaggagacagaataatagaaaagtcactgattggttaacattggattaagaattaaaacagaggaaactttaagattgaagtttgaaactgacttgtttgggaaatcaggctgtcttctttcttgatttcttagaaggccggataacaactgagttttgctttggtgaacatgggtgactccatttttacttttagtctggtctgttgaggcctcgtgagagagcttaatctaaaacaatgacttcctataatttttgtttgacacatccaaagagggactctaatatttattgagagcttatcatatcttaagtactgtttaaacacttttatttgctattacatttgatcttattataactctaaaggcagaaatgattgcttttattttccacaatggaggaaactgaggttcaattaagtgagtaaggaagcagggatcttaaacccagataccattgctcctctttaaaggtggaagaacagaaaacatggggcaggggaagagagaaagtttctgtcccaggacatgataatctaaaagggaaaacgtaagatccactgaaacctgaggcagatttattgtggcaataacaaagcttaagtttcacagaccttcatttgcctgagccaactttgaaggccatgtatctaattttgtttttataattctataatctttattcttgaaaagagccctccctccaaatttacaagctttgggcccccaaaatccttgaaatgcccttgaataagagatatccaggtaaatgctatgggaattcagaggaggaagcagttagtatcagttggcggagagttaggctattaagagaaggttttatataggaagtggcatttagaatgaagctttgagaactgagctgtgtatttgaacaagtaaaggtggtgttgcagaattttgctccttagttctattaaaaacccgggttcttgtcacatgatccggaaaatttaggcacacagatacattgaagcatgagtagagcaggattttattgggcaaaaaggaaaaaaagaaaactcagcaaatcgagatggagtcttgctcacagattgaatcccaggccaccacaaaggaactgaagagatcgggcttctcccctgcataaggtgcaaattccccatggctccacccacttccccttagtgtgcatgtggggctccagtccacggtgggcatgcccagacaagccttgggcaggttccctcatctgtgcaaaagcatctgatgtaaacacttgaggggtggttcggagattctctgggacccttttattttcttatctgcctaggcatttggctgtctcagtgggtgggaaagggtgctccaggcaaagggcataacatgaggcaaagggcatgcacagaaaacagtgactggttcagtcaggttgggggatgccaaaggaagtaatgggagacaagattggagcaagatagataagagattgtggattttttttcttttttatctatataaatacagagacagggtctcactatgttgcccaggctggtctcaaactcctggcctcaagtgatcctcccacctcatcctcccaaagtgctaggattacaggcatgaggcactgtgcccaacctccaattttggattttgagagctaaagcaatatagtcgaaaactcagataatccaggtagattttgctattaggtgctatttggttcctggtacagagctaaaacccttggaatttcctaagtgataagagctacaggagcatcttttgttatatgtttccccccctagttcctgaaatagctctagagaaatacaggtgaataacatcctttgttattcatatcaagcccctatcaaccataccccagtttctatttatgaagtggcttttgggaagtccctaaagacaggagtggggaaaggctggttgtcagggggatgggttgaaactttcatcttccccccttgacctccagggagggatgagtggctgaaaattgtgtaaaatcaacaatggccagtgatttaatcaaccatgcctatgtaatgaagccacccgataagccttaactggaactttttggagagcctccaggctggtgaagacattgaggtgctcagaaggtggtattccagagagagcacagaatctctgttccccttcccacattcattttgctatgcatctctcccatctggctgttcttgagaggtatccgtttataataaactggtaacctagtaagtaaactgttaccctgagttctgtgagccattctagcaaattatcaaacctaaagagttcatggatacgtgcaatttacagatgcacagtcagaagcacagatgacaatctgggcttgccattggcatttgaagtgtgttgggaggcagtcttacaggaatgagcccttatcctgtggggtctatgctaataacagacagttgtcagcattgcttggtgtcgaaaacccacattgttggtgtcagaagtattgtcagtaggatagggaaaacagtttgttttctttttttagtggtctttggtcatctttaagagcagggcttctcaaagtgtggtccttgaaccagcatcacctgtaccacgtaagaacttatgagaaatgttcattcttgggccccaacaaagaattaaaaattctgagggtgtgaacggggtctgagtttcagcacaacttcccgaccatgctgatgcattcttgcccaagcatgaaagccctcccttgtttaagaaggccattagggccgggtgtggtggctcatgcttgtaatcgagcactttgagaggacatagtgggaggatcacttgagccctggagttctagacaagcctgggcaacatggcaaaatgctgtctccacaaaaatcacaaaaattaggtgggcgtgtgttgtgtgcctataggcccagctacttaggagactgaggcaggaggatcgcttgagcccaggagattaaggctgcagcgagctgtgatggcaccactacagcctggatgacagagtgagacactgtctcaaaaaaaaaaaagaaaaagaaaaagaaaaaagaaaggaaaatgaaaaagaacgccattaggtataaaggagcaatggtaaaagaccagttgcaaaaggttagggaatgggtggttactgaaataagaagctatgtagaacactagtgttggtggcaggaagtagaaagcaagagcactgctctgtgggggatggtcatagcaaatgcaatatggaggcatttgcctctgcactgaggagaaaactatcttttccaagataggaggaaaggagataagtggaattaaagagaacctttgagcacagagttgggaaactgaaggtatttgtgttgtgctccctcaatcttttaattcaactataagctaaacccatgaaacttgagtagtttcagttatctgacttttttcttctcttttgatacagtgttggctattctgggtcttttgcctctctttatgtacttaagaatcagtttgccaatgtatgcaaaataactggctgggattttgattgtgattggcttgaatctatagatggagttgggaaggactgacatcttgacaatgttgaagcttcctattcatcattatgaaatatttctccatttgtttgattctttgatttcttttatcagaatttagttttcctcatatagtcttttaaaatattttgttatattttgttcaagtattttgtttttgaggaatgccaatgtaaatggtattgtgattttaatttcaaattccaatttttcattgctgttatataggaaaatgattttttttgcatgttagccttatatctttcaactttgctataatcaattattgatagtttcaaggattttttggtcaattattttgaatcttctacatagattatcatcatctgaacttagttttatttcttccttcccaatctgtatacctttatctccttttcttatttcattagctaggacttccagtatgatgttgaaagtagtggtgagaggggatatcttggtcttgttcttgatcttagtgggaaaacttcaagtttcttatcattaagtatgattttagctggagggtttttgtagaagttttttttttttaagttgaagaagtctccttctatttttagtttgctgatttttaaaaagaatcaggaatgggtgttaaattttgtgaaatgcttttctgcaactattgatttgagcactttatttttcttctttggcttgttgatgtgaagtacattaattgatttttgaatgctgaatcaaccttttgtacctgagattaatcccgtttggttgtggtatataattatttgtatacatgttgagttcgatttgctaatactttttgagaatttttgcattggtgttcatgaaaaaatattggtgtgtagttttttgtgacatctttatctgcttatggttttaaggtaatgctggcctcatagcatgagttagggagtatttcctctacttttacatttgagaagagattgcagagaattagtaaaattcctactttaaatattttgtggaattcaccagtgaacccatctggacctggtgctttctgttttggaaggtcattaattattttaaaatagatataggcctattcagattacctattttttctcatgcgagttttagcagattgtctttcaaggaattggtctatttcatttaggttatcaaatatgtcaacgtagagttattcatagtattcttttattatccttttaatgtgcaagggatctgtagtgatgtccccttttttgttttattgatattagcaatttgtgtcacatcttttattttgctttgttagccaggctagagatatctctatttttgatgtttttgatgaaccaactttttgttttattgattttctctgttgatttcgtgatttcaatttcatgatttttaaattatgcttacatttgatttaatttgatcttcttttgctagttatccaaggtggaagcttatattgttaagatccttttgcattcttatgcattcaatgatgtaaatttccctctaagcactgctttttctgcatctcacaaatattcatgagttgtattttcatgttcatttagtttgaaatatttttaaatttctcttgatatttctcttttgacccatgtgttacttagaagtgtgttgtttaatcaccatttttaaaaattttctagctatctttctgttattgatttctagtttaattccattgtggtctgagagcatatattgtataattttaatttttataaaatttgttaaggtgtgatttatggcccagaatgtggtctatcttggtgaatgttccatgtaagctttggaagactgtgtattctgctatatttgaatgaggtagtctatagacatcaattatgtccagttgattgatggtgctgttgaattcaactatgtccttactgattttccacctgctagatctgtccattctttgcagagggacactgaagtctccaactctagtagtgaatattctatttcttgttacagttttatcaacttctgcttcatgtcttttgatgctttgttgctagaaacatacacatgaagaattggtatgtcttttggagcatgacccatttatcctcatataatgcccctcattatttcctcgccctgatgtctgttctctctgaaagaaatatagcctctccaggtctcttttggttggtgttaaaatgacttaactttctttatcccccttacttttagtttatatgtggttttaaatttaaagtgggtttcttgtagacagcaaatagttcagagttgtttttcgatccactttgacaatctttgtcttttaattggtatatttggactattgatattttaagtgattattgatatagttagataaacatctactatatttattactgttttctgtctgttacactacttgttctttgtttatatttttattgtctactctttttctttccattgtggttttaatcgagcattttatatgtttccattttcttttcttagcatagtaattcttctttaaaaaaacattttttagtggttgcccctagagtttgcaatatacatttacaactaatctaagtccattttcaaataatactaaataatttcatgtgtagtgcaagtaccttttaataataaaacactcccagttccaccttccagtctcttgtattatagctataatttagttcacttacatatatgggtatacctaagtatatacattatcatatttatgattgaatatattgatgaaattattttgaaaaaactgttatcgttaaatcaattaagagtaagaaaaatagttctaattttattataaaatgaaataccttcatttattcattctctaatacactttctttctttatgtagatccaagtttctgacctgtataattttccttttctctcttcagcttctttgaacatttcttaccagccagacctactgacaacaattttccccaatttttgtttgtctgatagagactttatttcttcttgacttttgaagaataattccacagggcacagaactctagattggtgatttcttcccctcaaacccttaaatatttcattccactgccttcttgcttgcattgtttctgagaagttagatataattcttatctttgcctttctataggtaagatgttttttcctctggcttctatcaagattttttctttatgaacatgatatgcctttctttttgaacatgatatgcctttctttttgaacatgatatgcctttgtgtcggattttttttggcattattctgcttggttttctctgagtttcttggatatgtggtatggtatctgacactaatttggaaaaattctcagtcattattgcttcaaatatttcttctgttcttttttttcctttattctccttctggtattcccattacatgtatgttacagtttttgtagtcatcccgctgttttggatattctgtttttttcagtttttttttccttcgcatttcagtgttggaagtttctattgacatattctcaacctcagagattctttcttcagctgtgttcagtctaccaatgagtccatcaaaggcattttacatttttattacagaatttttgacctatagaatttcttttgattccatctttgaatctccatttctcttctgcttttcatctgttcttgcatgttgcctactttttccatgaaaacctttagctttttttttttttctttttgaggtggagtctcactgttgcccaggctggagtgcagtggtgtgatcttggctcactgcaacctctgcctcctgggttcaagtgattctcctcctcagcctcccaagtagctgggattacaggtgcctgccaccatgcctgagtaatttttgtatttttagtagagatggggttttatcatgttggccaggcgggtcttgaactcctaacctcaagtgatctgcccaccttagcctcccaaattgctgggattataggtgtgagccaccatgccctgcctttagcatgttaatcatagttgttttaaattcctgatctgttaattccaacatccctgtcatatctgactgtggttctgatgcttgctctgtgttttcaaatggtgtttttttttttttgccttttagtaagccttgtaattttttattgaaaggtggacatgatgtgctgggtaaaaggaactgtagtaaataggcctttagtaatgtactggtaggtgtagcagagggtgagggaagtattctgtagtcctatgattaggttttagtcttttagtgagcctgtgcgcctgcagcttggaagcacttgtgaagtgttttttcaccccttttggtgggacatagtgactagtgtgagcgggagttgagtatttcccttcccctaggtcagttaggctctgaaaaaaccctgataggttaggcatggtaaaatagtctcttttgagggcaggcattgttataagaatagaatgctctggggccaggtgcggtggctcacgcctgtaatccccgcactttgggaggctaaggcaggtggatcacctgaggtcaggagttcgagaccagcctggccaacatggtgaaaccccgtctctactaaaaatacaaaaatcagccaggtgtggtggcacacacctataatcccagctactcaggaggctgaggcaggagaactgcttgaacccagtaagtggaggttacagtgacccaagattgtgccactgcagtctagtctgggtgacagagcaagactccgtctcaaaaaaaaaagaatgctctggcatatttgaaaatggttacttttccctttttttctctgatcttcactgtgagaacctggtaagcatcctataggcaaaattcataaaagtatagaagtcggccagtgacttggacccacttggaattttcttgctctcacatcatgcacactgaatctccagcaatttttcacttacagtttaggttttcctaccctactactggttctctcagaggtttctgcttattggtttctgttttgtaagttgtgattctctgtacctaactgcctgtctcccattttggggggcagtggtttgccctgtgacctcacttctctgacagatctaagaaaagttgtttatttttcagtgtgctctgctttttacttgttacgatgaagccaaccactttcagaatttctacaaaccagatcagaatctggaagtcctgtttttttattttttttatccctttgtttagcatgttacctatcttaacacattttaaataagtgaatgcatagcttatatctacttctaggttatatgcttccttagaataggaattgattcttaaaatgtcgttctgctcacgcctgtaattccagcactttgggaggccaaggcaggcggatcacttggggtcaggagttcaagaccagcctggtcaacatggtaaaaccctgtgcctgcaaaaaatacaaaaattagctgggcatggtggtggccatctgtaatcccagctactagggaagctaaggcatgagaatcacttgaacctgggaggtggaggttgcagtgagctgagatcgcgccactgcactccagcctgggtgacaagagcaaaactccatctcataaataaataaataaataaataaataaataataaaaataaaaaaataaaataaaacaaaaattttattctgagcagtctctgaagaatataaattctactgccttgcctttagaacttataacagcatctcgcaaactatcacaagatgctccaaacatacttcttatgtgctgaattaagaagtcaactcaaatttagtatactagtaatatttttggatatcccaaaacactgccagctcagctttaggctgcccttcttgggggggaaaaaagcagttgaaatttaggacttaagtgggcatctcgtttaatttttaatggatttctatgttgttggttatggtgaagaggtgaaaagaataaatattctgtgcagaaaaattattcagtcttcatgtgaaaacactttgtccatagcaattactttatgaaaaagatgtggtattactttctttgctcttaactgagacctttaatttaaagaacctatactttacaagtttttattttcaatgcatgaaaaatgtagcagctatttcacaacctttacttttaaaatccatttttctttttaatctcaaatagttttttcttaaaaccttttgactttttatctaaattgtaatagccagagcaccttcccacaactagaatatctcatcctttttgtcttttctttttcctctcaaaatgcctactgggaacttaatttggagtcagattcttcatgataaatctggacttaatcaaaattcctcatatggtatattgtatatatcacagtactggatagtcctctgattaaatagatatttgatagtactttaaggtctatacttttggatgaacttaactgctttctccatttgtagtctcttgaaaatacagaaatttcagaaataatttataagaatatcaaggattcaaatcatatcagcacaaacacctaaatacttgtttgctttgttaaacacatatcccattttctatcttgataaacattggtgtaaagtagttgaatcattcagtgggtataagcagcatattctcaatactatgtttcattaataattaatagagatatatgaacacataaaagattcaattataatcaccttgtggatctaaatttcagttgacttgtcatcttgatttctggagaccacaaggtaatgaaaaataattacaagagtcttccatctgttgcagtattaaaatggcgagtaagacaccctgaaaggaaatgttctattcatggtacaatgcaattacagctagcaccaaattcaacactgtttaactttcaacatattattttgatttatcttgatccaacattctcagggaggaggtgcattgaagttattagaaaacactgacttagatttagggtatgtcttaaaagcttatttgcgggaagtactctagccttattcaacagatcactgagaagcctggaaaaacaaatcccggaaactaattattatgtgccagttatataaacaagaagactttgttgggtacaaaccagtgattccttgcctttgaaaaatgtgtcagatatcatgcattaccagcagttcaatgatataaggaaaccagagtaatagctaaaacctttaaagctaaaccaaagatttacaaattgcctcttcatccagtctttcccaacctaaaaactgagttctctaaaaattttagtatttttttctgaagaaaagggaacatggacatttatctaatcctcattagaaatctgactaatgataacaaggatttagacctcaagcacttcttaccaaaattcttgatatgaccttatagcaaattactttcacctgttgaactttcctttcttttattcccctgtacctcacctgcactgggcatattcaagttgcttatacaacactttactattgtgttagaaaaatcatgacacatgatgaatgtgtttgtgcaacatgagctgattcataaatgaaaatgtgcattgaaattccacaatattttaaaattaggagtttatctagcaattgaacaaaattgattaaatccattatttgttagatcagctaaattacataagttcattcatctgctcataaatccatccattcttccatctggctatcccttagtcaattcaaataaatatttatggggcactttgggtaagccaggtgctaagaattcaatgcaaaacaagatagactcccctgtccttgttgaacttatatttttggtacaaacaaaagcaataatcaagaaaaaataaaaaaagtactgattgtgattaataatatgaagaaattcaacagagtattgtacttaacatttgattgatctgattttctcagttgtctgagaacaaacatttgtgaaaatctcattgtagagttcttacgatggatagggggtcaactgtgtcattattgcttatcagcttatcccaaagacctagtttattaccagattgcaaatagtgttcaataaattattcttattaagggttgttatgtactctaaaacatttattgtggtcccttcactggttctggtttacaaacttacttttctatgatgacatagtatagaaattgagagtgaatatttagaagttcatttttattatatatttttgaagtattgatatgtagtgaattagaaatttaaaaagaaaacaaaactgtccttcactacagattgaaaagcattatactaaaagaccatttgctcagttatagtatataaaggccaaatgacttaaaaacaaattatgtaaggagaaggaaacaaccatttattcagtgccactaactgtcagccagttttttcagtggtcagttaatgactgcagtagtgttctaccttgctcaaagcaccctcctcaagttctggcatctaagctgacatcagaacacagagttggggctctctgtgggtcacctctagcacttgatctcctcatgcagtgcatggtgctctcacgtctatgctatgttcttatggtctttaggtaacaagaataattttctttcttttccttactatacattttgctttctgaaattcccttctcgccaatccaggtgaatgtcagaatgtgatttgacaactgtccaaagtactcattcactgaggagtggtaaggccttcgcccaacctgccttctctgggaatatactgctgcctgaacatatcattgtttattgccaggcttgaacttcaccaaattaatttattagggtcaacatctaaatattagaactatttcagattaatttttaagtcgtatccactttgggtactagatcaaattgcaggtctctgcttctggcttgagcctatgtttagagatgatgtgcatgaagacactctttgcttttcctttatgcaaaatgggcattttcaatctttttgtcattagtaaaggtcagtgataaaggaagtctgcatcaggggtccaattccttatggccagtttctctattctgttccaaggttgtttgtctccatatatcaacattggtcaggattgaaagtgtgcaacaaggtttgaatgaataagtgaaaatcttccactggtgacaggataaaatattccaatggtttttattgaagtacaatactgaattatgtttatggcatggtacctatatgtcacagaagtgatcccatcacttttaccttatag

SEQ ID NO: 71 - DNA, 14667 bp, Homo sapiens
  misc_feature (1)..(14667): CFTR exon 19 containing 3849 + 10 kb C-to-T mutation
  misc_feature (12191)..(12191): 3849 + 10 kb C-to-T mutation
gtgagatttgaacactgcttgctttgttagactgtgttcagtaagtgaatcccagtagcctgaagcaatgtgttagcagaatctatttgtaacattattattgtacagtagaatcaatattaaacacacatgttttattatatggagtcattatttttaatatgaaatttaatttgcagagtcctgaacctatataatgggtttattttaaatgtgattgtacttgcagaatatctaattaattgctaggttaataactaaagaagccattaaataaatcaaaattgtaacatgttttagatttcccatcttgaaaatgtcttccaaaaatatcttattgctgactccatctattgtcttaaattttatctaagttccattctgccaaacaagtgatactttttttctagcttttttcagtttgtttgttttgtttttctttgaagttttaattcagacatagattattttttcccagttatttactatatttattaagcatgagtaattgacattattttgaaatccttcttatggatcccagcactgggctgaacacatagaaggaacttaatatatactgatttctggaattgattcttggagacagggatggtcattatccatatacttcaggctccataaacatatttcttaattgccttcaaatccctattctggactgctctataaatctagacaagagtattatatattttgattgatattttttagataaaataaaagggagctgaaaactgaattgcaaactgaattttaaaactttatctctctgtggttaattgcaaacacagatacaaaaatatagagagagatacagttagtaaagatgttaggtcaccgttactaacactgacatagaaacagttttgctcatgagtttcagaatatatgagtttgattttgcccatggattttagaatatttgataaacatttaatgcattgtacaaattctgtgaaaacatatatataggatgtgcgaaaagtccctgtgtatcatgtgaaatggcttaaaacagaacaccataggtattcatatcagtgaataccataggtagctgaaagtgttttttcctggggtcgccaagatgaatgccaaaagtgatatcattattataaacaatagccagaataggttggtataaacctggtagaaagccttgataaattgactttctctcctcctgacatcctgccacccctttgctttgctgatgctcatttgtccactaaattaaactcaagcaagccctagtaaagtaatagaatttgtggagtcctcattagtataggaagtttccctgatgtgagattagtaattagagatgtagcaaaatgagaaagaagtaatatgcttagatatttcattttctctgaacctgtatatacaaaataggccatgcgtgttcagtaactattcactgcaaggcactctctaggtactttgggggaattggaaattactcacataaggctatggattgtgccatttgtcaaaagacaaaatgacaacaaatttagtttaaagacctcagtcagctttattttctattctagatttggacagtccttcatttcacaaattggagtaagtgttccaataagttgagcaaaggagcttggctttatagacccaaaaaaagggccaaaggaagcagaaacaaagaacaataagagaattggtcatttcaaagttacttttcttgaaaggtggggacaaggagacagaataatagaaaagtcactgattggttaacattggattaagaattaaaacagaggaaactttaagattgaagtttgaaactgacttgtttgggaaatcaggctgtcttctttcttgatttcttagaaggccggataacaactgagttttgctttggtgaacatgggtgactccatttttacttttagtctggtctgttgaggcctcgtgagagagcttaatctaaaacaatgacttcctataatttttgtttgacacatccaaagagggactctaatatttattgagagcttatcatatcttaagtactgtttaaacacttttatttgctattacatttgatcttattataactctaaaggcagaaatgattgcttttattttccacaatggaggaaactgaggttcaattaagtgagtaaggaagcagggatcttaaacccagataccattgctcctctttaaaggtggaagaacagaaaacatggggcaggggaagagagaaagtttctgtcccaggacatgataatctaaaagggaaaacgtaagatccactgaaacctgaggcagatttattgtggcaataacaaagcttaagtttcacagaccttcatttgcctgagccaactttgaaggccatgtatctaattttgtttttataattctataatctttattcttgaaaagagccctccctccaaatttacaagctttgggcccccaaaatccttgaaatgcccttgaataagagatatccaggtaaatgctatgggaattcagaggaggaagcagttagtatcagttggcggagagttaggctattaagagaaggttttatataggaagtggcatttagaatgaagctttgagaactgagctgtgtatttgaacaagtaaaggtggtgttgcagaattttgctccttagttctattaaaaacccgggttcttgtcacatgatccggaaaatttaggcacacagatacattgaagcatgagtagagcaggattttattgggcaaaaaggaaaaaaagaaaactcagcaaatcgagatggagtcttgctcacagattgaatcccaggccaccacaaaggaactgaagagatcgggcttctcccctgcataaggtgcaaattccccatggctccacccacttccccttagtgtgcatgtggggctccagtccacggtgggcatgcccagacaagccttgggcaggttccctcatctgtgcaaaagcatctgatgtaaacacttgaggggtggttcggagattctctgggacccttttattttcttatctgcctaggcatttggctgtctcagtgggtgggaaagggtgctccaggcaaagggcataacatgaggcaaagggcatgcacagaaaacagtgactggttcagtcaggttgggggatgccaaaggaagtaatgggagacaagattggagcaagatagataagagattgtggattttttttcttttttatctatataaatacagagacagggtctcactatgttgcccaggctggtctcaaactcctggcctcaagtgatcctcccacctcatcctcccaaagtgctaggattacaggcatgaggcactgtgcccaacctccaattttggattttgagagctaaagcaatatagtcgaaaactcagataatccaggtagattttgctattaggtgctatttggttcctggtacagagctaaaacccttggaatttcctaagtgataagagctacaggagcatcttttgttatatgtttccccccctagttcctgaaatagctctagagaaatacaggtgaataacatcctttgttattcatatcaagcccctatcaaccataccccagtttctatttatgaagtggcttttgggaagtccctaaagacaggagtggggaaaggctggttgtcagggggatgggttgaaactttcatcttccccccttgacctccagggagggatgagtggctgaaaattgtgtaaaatcaacaatggccagtgatttaatcaaccatgcctatgtaatgaagccacccgataagccttaactggaactttttggagagcctccaggctggtgaagacattgaggtgctcagaaggtggtattccagagagagcacagaatctctgttccccttcccacattcattttgctatgcatctctcccatctggctgttcttgagaggtatccgtttataataaactggtaacctagtaagtaaactgttaccctgagttctgtgagccattctagcaaattatcaaacctaaagagttcatggatacgtgcaatttacagatgcacagtcagaagcacagatgacaatctgggcttgccattggcatttgaagtgtgttgggaggcagtcttacaggaatgagcccttatcctgtggggtctatgctaataacagacagttgtcagcattgcttggtgtcgaaaacccacattgttggtgtcagaagtattgtcagtaggatagggaaaacagtttgttttctttttttagtggtctttggtcatctttaagagcagggcttctcaaagtgtggtccttgaaccagcatcacctgtaccacgtaagaacttatgagaaatgttcattcttgggccccaacaaagaattaaaaattctgagggtgtgaacggggtctgagtttcagcacaacttcccgaccatgctgatgcattcttgcccaagcatgaaagccctcccttgtttaagaaggccattagggccgggtgtggtggctcatgcttgtaatcgagcactttgagaggacatagtgggaggatcacttgagccctggagttctagacaagcctgggcaacatggcaaaatgctgtctccacaaaaatcacaaaaattaggtgggcgtgtgttgtgtgcctataggcccagctacttaggagactgaggcaggaggatcgcttgagcccaggagattaaggctgcagcgagctgtgatggcaccactacagcctggatgacagagtgagacactgtctcaaaaaaaaaaaagaaaaagaaaaagaaaaaagaaaggaaaatgaaaaagaacgccattaggtataaaggagcaatggtaaaagaccagttgcaaaaggttagggaatgggtggttactgaaataagaagctatgtagaacactagtgttggtggcaggaagtagaaagcaagagcactgctctgtgggggatggtcatagcaaatgcaatatggaggcatttgcctctgcactgaggagaaaactatcttttccaagataggaggaaaggagataagtggaattaaagagaacctttgagcacagagttgggaaactgaaggtatttgtgttgtgctccctcaatcttttaattcaactataagctaaacccatgaaacttgagtagtttcagttatctgacttttttcttctcttttgatacagtgttggctattctgggtcttttgcctctctttatgtacttaagaatcagtttgccaatgtatgcaaaataactggctgggattttgattgtgattggcttgaatctatagatggagttgggaaggactgacatcttgacaatgttgaagcttcctattcatcattatgaaatatttctccatttgtttgattctttgatttcttttatcagaatttagttttcctcatatagtcttttaaaatattttgttatattttgttcaagtattttgtttttgaggaatgccaatgtaaatggtattgtgattttaatttcaaattccaatttttcattgctgttatataggaaaatgattttttttgcatgttagccttatatctttcaactttgctataatcaattattgatagtttcaaggattttttggtcaattattttgaatcttctacatagattatcatcatctgaacttagttttatttcttccttcccaatctgtatacctttatctccttttcttatttcattagctaggacttccagtatgatgttgaaagtagtggtgagaggggatatcttggtcttgttcttgatcttagtgggaaaacttcaagtttcttatcattaagtatgattttagctggagggtttttgtagaagttttttttttttaagttgaagaagtctccttctatttttagtttgctgatttttaaaaagaatcaggaatgggtgttaaattttgtgaaatgcttttctgcaactattgatttgagcactttatttttcttctttggcttgttgatgtgaagtacattaattgatttttgaatgctgaatcaaccttttgtacctgagattaatcccgtttggttgtggtatataattatttgtatacatgttgagttcgatttgctaatactttttgagaatttttgcattggtgttcatgaaaaaatattggtgtgtagttttttgtgacatctttatctgcttatggttttaaggtaatgctggcctcatagcatgagttagggagtatttcctctacttttacatttgagaagagattgcagagaattagtaaaattcctactttaaatattttgtggaattcaccagtgaacccatctggacctggtgctttctgttttggaaggtcattaattattttaaaatagatataggcctattcagattacctattttttctcatgcgagttttagcagattgtctttcaaggaattggtctatttcatttaggttatcaaatatgtcaacgtagagttattcatagtattcttttattatccttttaatgtgcaagggatctgtagtgatgtccccttttttgttttattgatattagcaatttgtgtcacatcttttattttgctttgttagccaggctagagatatctctatttttgatgtttttgatgaaccaactttttgttttattgattttctctgttgatttcgtgatttcaatttcatgatttttaaattatgcttacatttgatttaatttgatcttcttttgctagttatccaaggtggaagcttatattgttaagatccttttgcattcttatgcattcaatgatgtaaatttccctctaagcactgctttttctgcatctcacaaatattcatgagttgtattttcatgttcatttagtttgaaatatttttaaatttctcttgatatttctcttttgacccatgtgttacttagaagtgtgttgtttaatcaccatttttaaaaattttctagctatctttctgttattgatttctagtttaattccattgtggtctgagagcatatattgtataattttaatttttataaaatttgttaaggtgtgatttatggcccagaatgtggtctatcttggtgaatgttccatgtaagctttggaagactgtgtattctgctatatttgaatgaggtagtctatagacatcaattatgtccagttgattgatggtgctgttgaattcaactatgtccttactgattttccacctgctagatctgtccattctttgcagagggacactgaagtctccaactctagtagtgaatattctatttcttgttacagttttatcaacttctgcttcatgtcttttgatgctttgttgctagaaacatacacatgaagaattggtatgtcttttggagcatgacccatttatcctcatataatgcccctcattatttcctcgccctgatgtctgttctctctgaaagaaatatagcctctccaggtctcttttggttggtgttaaaatgacttaactttctttatcccccttacttttagtttatatgtggttttaaatttaaagtgggtttcttgtagacagcaaatagttcagagttgtttttcgatccactttgacaatctttgtcttttaattggtatatttggactattgatattttaagtgattattgatatagttagataaacatctactatatttattactgttttctgtctgttacactacttgttctttgtttatatttttattgtctactctttttctttccattgtggttttaatcgagcattttatatgtttccattttcttttcttagcatagtaattcttctttaaaaaaacattttttagtggttgcccctagagtttgcaatatacatttacaactaatctaagtccattttcaaataatactaaataatttcatgtgtagtgcaagtaccttttaataataaaacactcccagttccaccttccagtctcttgtattatagctataatttagttcacttacatatatgggtatacctaagtatatacattatcatatttatgattgaatatattgatgaaattattttgaaaaaactgttatcgttaaatcaattaagagtaagaaaaatagttctaattttattataaaatgaaataccttcatttattcattctctaatacactttctttctttatgtagatccaagtttctgacctgtataattttccttttctctcttcagcttctttgaacatttcttaccagccagacctactgacaacaattttccccaatttttgtttgtctgatagagactttatttcttcttgacttttgaagaataattccacagggcacagaactctagattggtgatttcttcccctcaaacccttaaatatttcattccactgccttcttgcttgcattgtttctgagaagttagatataattcttatctttgcctttctataggtaagatgttttttcctctggcttctatcaagattttttctttatgaacatgatatgcctttctttttgaacatgatatgcctttctttttgaacatgatatgcctttgtgtcggattttttttggcattattctgcttggttttctctgagtttcttggatatgtggtatggtatctgacactaatttggaaaaattctcagtcattattgcttcaaatatttcttctgttcttttttttcctttattctccttctggtattcccattacatgtatgttacagtttttgtagtcatcccgctgttttggatattctgtttttttcagtttttttttccttcgcatttcagtgttggaagtttctattgacatattctcaacctcagagattctttcttcagctgtgttcagtctaccaatgagtccatcaaaggcattttacatttttattacagaatttttgacctatagaatttcttttgattccatctttgaatctccatttctcttctgcttttcatctgttcttgcatgttgcctactttttccatgaaaacctttagctttttttttttttctttttgaggtggagtctcactgttgcccaggctggagtgcagtggtgtgatcttggctcactgcaacctctgcctcctgggttcaagtgattctcctcctcagcctcccaagtagctgggattacaggtgcctgccaccatgcctgagtaatttttgtatttttagtagagatggggttttatcatgttggccaggcgggtcttgaactcctaacctcaagtgatctgcccaccttagcctcccaaattgctgggattataggtgtgagccaccatgccctgcctttagcatgttaatcatagttgttttaaattcctgatctgttaattccaacatccctgtcatatctgactgtggttctgatgcttgctctgtgttttcaaatggtgtttttttttttttgccttttagtaagccttgtaattttttattgaaaggtggacatgatgtgctgggtaaaaggaactgtagtaaataggcctttagtaatgtactggtaggtgtagcagagggtgagggaagtattctgtagtcctatgattaggttttagtcttttagtgagcctgtgcgcctgcagcttggaagcacttgtgaagtgttttttcaccccttttggtgggacatagtgactagtgtgagcgggagttgagtatttcccttcccctaggtcagttaggctctgaaaaaaccctgataggttaggcatggtaaaatagtctcttttgagggcaggcattgttataagaatagaatgctctggggccaggtgcggtggctcacgcctgtaatccccgcactttgggaggctaaggcaggtggatcacctgaggtcaggagttcgagaccagcctggccaacatggtgaaaccccgtctctactaaaaatacaaaaatcagccaggtgtggtggcacacacctataatcccagctactcaggaggctgaggcaggagaactgcttgaacccagtaagtggaggttacagtgacccaagattgtgccactgcagtctagtctgggtgacagagcaagactccgtctcaaaaaaaaaagaatgctctggcatatttgaaaatggttacttttccctttttttctctgatcttcactgtgagaacctggtaagcatcctataggcaaaattcataaaagtatagaagtcggccagtgacttggacccacttggaattttcttgctctcacatcatgcacactgaatctccagcaatttttcacttacagtttaggttttcctaccctactactggttctctcagaggtttctgcttattggtttctgttttgtaagttgtgattctctgtacctaactgcctgtctcccattttggggggcagtggtttgccctgtgacctcacttctctgacagatctaagaaaagttgtttatttttcagtgtgctctgctttttacttgttacgatgaagccaaccactttcagaatttctacaaaccagatcagaatctggaagtcctgtttttttattttttttatccctttgtttagcatgttacctatcttaacacattttaaataagtgaatgcatagcttatatctacttctaggttatatgcttccttagaataggaattgattcttaaaatgtcgttctgctcacgcctgtaattccagcactttgggaggccaaggcaggcggatcacttggggtcaggagttcaagaccagcctggtcaacatggtaaaaccctgtgcctgcaaaaaatacaaaaattagctgggcatggtggtggccatctgtaatcccagctactagggaagctaaggcatgagaatcacttgaacctgggaggtggaggttgcagtgagctgagatcgcgccactgcactccagcctgggtgacaagagcaaaactccatctcataaataaataaataaataaataaataaataataaaaataaaaaaataaaataaaacaaaaattttattctgagcagtctctgaagaatataaattctactgccttgcctttagaacttataacagcatctcgcaaactatcacaagatgctccaaacatacttcttatgtgctgaattaagaagtcaactcaaatttagtatactagtaatatttttggatatcccaaaacactgccagctcagctttaggctgcccttcttgggggggaaaaaagcagttgaaatttaggacttaagtgggcatctcgtttaatttttaatggatttctatgttgttggttatggtgaagaggtgaaaagaataaatattctgtgcagaaaaattattcagtcttcatgtgaaaacactttgtccatagcaattactttatgaaaaagatgtggtattactttctttgctcttaactgagacctttaatttaaagaacctatactttacaagtttttattttcaatgcatgaaaaatgtagcagctatttcacaacctttacttttaaaatccatttttctttttaatctcaaatagttttttcttaaaaccttttgactttttatctaaattgtaatagccagagcaccttcccacaactagaatatctcatcctttttgtcttttctttttcctctcaaaatgcctactgggaacttaatttggagtcagattcttcatgataaatctggacttaatcaaaattcctcatatggtatattgtatatatcacagtactggatagtcctctgattaaatagatatttgatagtactttaaggtctatacttttggatgaacttaactgctttctccatttgtagtctcttgaaaatacagaaatttcagaaataatttataagaatatcaaggattcaaatcatatcagcacaaacacctaaatacttgtttgctttgttaaacacatatcccattttctatcttgataaacattggtgtaaagtagttgaatcattcagtgggtataagcagcatattctcaatactatgtttcattaataattaatagagatatatgaacacataaaagattcaattataatcaccttgtggatctaaatttcagttgacttgtcatcttgatttctggagaccacaaggtaatgaaaaataattacaagagtcttccatctgttgcagtattaaaatggtgagtaagacaccctgaaaggaaatgttctattcatggtacaatgcaattacagctagcaccaaattcaacactgtttaactttcaacatattattttgatttatcttgatccaacattctcagggaggaggtgcattgaagttattagaaaacactgacttagatttagggtatgtcttaaaagcttatttgcgggaagtactctagccttattcaacagatcactgagaagcctggaaaaacaaatcccggaaactaattattatgtgccagttatataaacaagaagactttgttgggtacaaaccagtgattccttgcctttgaaaaatgtgtcagatatcatgcattaccagcagttcaatgatataaggaaaccagagtaatagctaaaacctttaaagctaaaccaaagatttacaaattgcctcttcatccagtctttcccaacctaaaaactgagttctctaaaaattttagtatttttttctgaagaaaagggaacatggacatttatctaatcctcattagaaatctgactaatgataacaaggatttagacctcaagcacttcttaccaaaattcttgatatgaccttatagcaaattactttcacctgttgaactttcctttcttttattcccctgtacctcacctgcactgggcatattcaagttgcttatacaacactttactattgtgttagaaaaatcatgacacatgatgaatgtgtttgtgcaacatgagctgattcataaatgaaaatgtgcattgaaattccacaatattttaaaattaggagtttatctagcaattgaacaaaattgattaaatccattatttgttagatcagctaaattacataagttcattcatctgctcataaatccatccattcttccatctggctatcccttagtcaattcaaataaatatttatggggcactttgggtaagccaggtgctaagaattcaatgcaaaacaagatagactcccctgtccttgttgaacttatatttttggtacaaacaaaagcaataatcaagaaaaaataaaaaaagtactgattgtgattaataatatgaagaaattcaacagagtattgtacttaacatttgattgatctgattttctcagttgtctgagaacaaacatttgtgaaaatctcattgtagagttcttacgatggatagggggtcaactgtgtcattattgcttatcagcttatcccaaagacctagtttattaccagattgcaaatagtgttcaataaattattcttattaagggttgttatgtactctaaaacatttattgtggtcccttcactggttctggtttacaaacttacttttctatgatgacatagtatagaaattgagagtgaatatttagaagttcatttttattatatatttttgaagtattgatatgtagtgaattagaaatttaaaaagaaaacaaaactgtccttcactacagattgaaaagcattatactaaaagaccatttgctcagttatagtatataaaggccaaatgacttaaaaacaaattatgtaaggagaaggaaacaaccatttattcagtgccactaactgtcagccagttttttcagtggtcagttaatgactgcagtagtgttctaccttgctcaaagcaccctcctcaagttctggcatctaagctgacatcagaacacagagttggggctctctgtgggtcacctctagcacttgatctcctcatgcagtgcatggtgctctcacgtctatgctatgttcttatggtctttaggtaacaagaataattttctttcttttccttactatacattttgctttctgaaattcccttctcgccaatccaggtgaatgtcagaatgtgatttgacaactgtccaaagtactcattcactgaggagtggtaaggccttcgcccaacctgccttctctgggaatatactgctgcctgaacatatcattgtttattgccaggcttgaacttcaccaaattaatttattagggtcaacatctaaatattagaactatttcagattaatttttaagtcgtatccactttgggtactagatcaaattgcaggtctctgcttctggcttgagcctatgtttagagatgatgtgcatgaagacactctttgcttttcctttatgcaaaatgggcattttcaatctttttgtcattagtaaaggtcagtgataaaggaagtctgcatcaggggtccaattccttatggccagtttctctattctgttccaaggttgtttgtctccatatatcaacattggtcaggattgaaagtgtgcaacaaggtttgaatgaataagtgaaaatcttccactggtgacaggataaaatattccaatggtttttattgaagtacaatactgaattatgtttatggcatggtacctatatgtcacagaagtgatcccatcacttttaccttatag

SEQ ID NO: 72 - DNA, 18 bp, Artificial
  CFTR exon 19 wild-type oligo
gtcttactcgccatttta

SEQ ID NO: 73 - DNA, 18 bp, Artificial
  CFTR exon 19 3849 + 10 kb C-to-T mutation oligo
  misc_feature (10)..(10): 3849 + 10 kb C-to-T mutation
gtcttactcaccatttta

SEQ ID NO: 74 - DNA, 3733 bp, Mus musculus
  misc_feature (1)..(3733): wild-type Mus musculus dystrophin intron 22, exon 23 and intron 23 sequences
  Intron (1)..(913): intron 22
  exon (914)..(1126): exon 23
  Intron (1127)..(3733): intron 23
gtctgtggacatttgaatatcataaataacaaagaacatgtcttatcagtcaagagatcatattgatatattaaacttaaggtaataatgaaaaagtaaagataataatgaaaaatcatagattatgagttggaaaaataaacagaacaatttgaccaaaaacatgactttttcttatttttttctatatattattttataaatatacagacataaatagatatatatttttaaattaaaagtactgtattaaaggaaaggtataatttcatttcatatttagtgacataagatatgaagtatgattattaaaattaaatcacattattttattataattactttatttttaattcctaatttctttaagcttaggtaaaatcaatggatttatataattagttagaatttaaatattaacaaactataacactatgattaaatgcttgatattgagtagttattttaatagcctaagtctggaaattaaatactagtaagagaaacttctgtgatgtgaggacatataaagactaatttttttgttgattctaaaaatcccatgttgtatacttattctttttaaatctgaaaatatattaatcatatattgcctaaatgtcttaataatgtttcactgtaggtaagttaaaatgtatcacatatataataaacatagttattaatgcatagatattcagtaaaattatgacttctaaatttctgtctaaatataatatgccctgtaatataatagaaattattcataagaatacatatatattgctttatcagatattctactttgtttagatctctaaattacataaacttttatttaccttcttcttgatatgaatgaaactcatcaaatatgcgtgttagtgtaaatgaacttctatttaattttgaggctctgcaaagttctttgaaagagcaacaaaatggcttcaactatctgagtgacactgtgaaggagatggccaagaaagcaccttcagaaatatgccagaaatatctgtcagaatttgaagagattgaggggcactggaagaaactttcctcccagttggtggaaagctgccaaaagctagaagaacatatgaataaacttcgaaaatttcaggtaagccgaggtttggcctttaaactatattttttcacatagcaattaattggaaaatgtgatgggaaacagatattttacccagagtccttcaaagatattgatgatatcaaaagccaaatctatttcaaaggattgcaacttgcctatttttcctatgaaaacagtaatgtgtcataccttcttggattgtctgtataaatgaattgattttttttcaccaactccaagtatacttaacattttaacataataatttaaaatatccttattccattatgttcattttttaagttgtagatatgatttagctcacagcatacatatatacacatgtattacatatgcatatattatatatatggcagacatatgttttcactaccatatttcacttttgaattatgaatatatgtttaatttctgccatatttccttccctacattgacttctattaatttagtatttcagtagttctaacacattaataataacctagactcaatacagtaatctaacaattatatttgtgcctgtaattctaagttagttaaattcataggttgtgtttctcatagttggccatttgtgaaatataataatatccgaaaagaaagttcaaaaatgtcatgacttcatatagagttattgaaacagtgcccttactttcattctggccatgctagtgacttgatcattcttgtattttacagctaaaacactaccaaaagtgtcaaatccatgatctacatgtttgactgaggctagcagcacttattccacccttatatgaagcctttaagagaaagtatatttgtttgctatttttaacttcttgaaggaacatacaatctttgtttcaagagctcatcctctttcatgctagtaaattttggtggcattgcatccatgtctgactctgaatctgtttctgtctatcctgctccctaacactgtaccatcttcctttttgaaaaaaaaatattgaattattttatttatttactttccaaagttgctcctgcctgttcctccttctccaagttcttcagtcccccctgctccccaccgatgagagggaaaggtcctgaattcactgggctccatgggggtccttttgcattttcttaaccttcttaataaaataggccttctagaattatatcatatacattgtgatatgacaaatgataaagtatattgttcagagttttaccttgttcatatttgcaatgtccccctgtcatgctggatattctttgattgggtatatttgctaacagattaagtatatttatcttcgttaagcagtataacttattaagaaagaactctattaatatgagaaataactaatgaaacaccactccacaggtgatttcagccactttatgaactgctggaagcaaaaatgagatctttgcaacatgaagcagttgctcagttcattaaactgtgttcaatatttcagccataacatacattagagaatgatttatattgttcaaacatttggtgctctatttttgcatgacgtgggattaaacacagcaccaacaatcaaacaattgcaaagatgtattacaagtattttttctttttaaaacaggaaagtatacttatatttccattgtccaaaccatcatgaaagggatagagattactgacacaaatttagagaaaggatttgagtggagtaagaattaaatgaaccaaagaagaattaatgtattcatcaagaagtcatggaggtgaaattggccttgaatgataccactaaggagagaatgttgagatccttatatttagtcaattgtttttaaatctgtagttattaaccacattttaatcatattgaaagggaaattttctgtgatgcatgtattttcaatataaattttagaaaagaagacaattataacttgattttgtgaattacatggaactaaagaaatgacagatttacatttgaaaattgactgaactaaagtacataaataaaagtcatacagaaaaatgtgggaggtgcttgtccatttataaaggacaaaaatgccatttgttgcctaatcattatttcttattggtcagaccaataagaaatcaagagctttgactttaaaggtaagaaaatcttaccttaaaatccccaactgaagggactgtttaaactgtcaactgcagaaaacaagttatggaagttcaggtttagggaaactataaacacaccataacattgagtttatgtgcatagtttgttttatgtacagtgagagtaaattgttagtattatcatgagttgttttgaaacttcaaatttctctagaggggtatgatttaatgttctcaagaggaacataataaaaccatatctggtattagtttttatttttaacaatagcagacttcatacaccaatgttcacagtgtagaccataaaatgcagtcttagtaaaaatattattctctataaagctacaatgagacctccctcaaacatacattgtttttttttttctaacttatgtttggatatatcatcatgatgaactatgttaaaaacaatcagagcttagtaatactttcatattgcttttttattccag

SEQ ID NO: 75 - DNA, 3733 bp, Mus musculus
  misc_feature (1)..(3733): mdx Mus musculus dystrophin intron 22, exon 23 and intron 23 sequences
  Intron (1)..(913): intron 22
  exon (914)..(1126): exon 23
  misc_feature (941)..(941): mdx C to T nonsense mutation
  Intron (1127)..(3733): intron 23
gtctgtggacatttgaatatcataaataacaaagaacatgtcttatcagtcaagagatcatattgatatattaaacttaaggtaataatgaaaaagtaaagataataatgaaaaatcatagattatgagttggaaaaataaacagaacaatttgaccaaaaacatgactttttcttatttttttctatatattattttataaatatacagacataaatagatatatatttttaaattaaaagtactgtattaaaggaaaggtataatttcatttcatatttagtgacataagatatgaagtatgattattaaaattaaatcacattattttattataattactttatttttaattcctaatttctttaagcttaggtaaaatcaatggatttatataattagttagaatttaaatattaacaaactataacactatgattaaatgcttgatattgagtagttattttaatagcctaagtctggaaattaaatactagtaagagaaacttctgtgatgtgaggacatataaagactaatttttttgttgattctaaaaatcccatgttgtatacttattctttttaaatctgaaaatatattaatcatatattgcctaaatgtcttaataatgtttcactgtaggtaagttaaaatgtatcacatatataataaacatagttattaatgcatagatattcagtaaaattatgacttctaaatttctgtctaaatataatatgccctgtaatataatagaaattattcataagaatacatatatattgctttatcagatattctactttgtttagatctctaaattacataaacttttatttaccttcttcttgatatgaatgaaactcatcaaatatgcgtgttagtgtaaatgaacttctatttaattttgaggctctgcaaagttctttgaaagagcaataaaatggcttcaactatctgagtgacactgtgaaggagatggccaagaaagcaccttcagaaatatgccagaaatatctgtcagaatttgaagagattgaggggcactggaagaaactttcctcccagttggtggaaagctgccaaaagctagaagaacatatgaataaacttcgaaaatttcaggtaagccgaggtttggcctttaaactatattttttcacatagcaattaattggaaaatgtgatgggaaacagatattttacccagagtccttcaaagatattgatgatatcaaaagccaaatctatttcaaaggattgcaacttgcctatttttcctatgaaaacagtaatgtgtcataccttcttggattgtctgtataaatgaattgattttttttcaccaactccaagtatacttaacattttaacataataatttaaaatatccttattccattatgttcattttttaagttgtagatatgatttagctcacagcatacatatatacacatgtattacatatgcatatattatatatatggcagacatatgttttcactaccatatttcacttttgaattatgaatatatgtttaatttctgccatatttccttccctacattgacttctattaatttagtatttcagtagttctaacacattaataataacctagactcaatacagtaatctaacaattatatttgtgcctgtaattctaagttagttaaattcataggttgtgtttctcatagttggccatttgtgaaatataataatatccgaaaagaaagttcaaaaatgtcatgacttcatatagagttattgaaacagtgcccttactttcattctggccatgctagtgacttgatcattcttgtattttacagctaaaacactaccaaaagtgtcaaatccatgatctacatgtttgactgaggctagcagcacttattccacccttatatgaagcctttaagagaaagtatatttgtttgctatttttaacttcttgaaggaacatacaatctttgtttcaagagctcatcctctttcatgctagtaaattttggtggcattgcatccatgtctgactctgaatctgtttctgtctatcctgctccctaacactgtaccatcttcctttttgaaaaaaaaatattgaattattttatttatttactttccaaagttgctcctgcctgttcctccttctccaagttcttcagtcccccctgctccccaccgatgagagggaaaggtcctgaattcactgggctccatgggggtccttttgcattttcttaaccttcttaataaaataggccttctagaattatatcatatacattgtgatatgacaaatgataaagtatattgttcagagttttaccttgttcatatttgcaatgtccccctgtcatgctggatattctttgattgggtatatttgctaacagattaagtatatttatcttcgttaagcagtataacttattaagaaagaactctattaatatgagaaataactaatgaaacaccactccacaggtgatttcagccactttatgaactgctggaagcaaaaatgagatctttgcaacatgaagcagttgctcagttcattaaactgtgttcaatatttcagccataacatacattagagaatgatttatattgttcaaacatttggtgctctatttttgcatgacgtgggattaaacacagcaccaacaatcaaacaattgcaaagatgtattacaagtattttttctttttaaaacaggaaagtatacttatatttccattgtccaaaccatcatgaaagggatagagattactgacacaaatttagagaaaggatttgagtggagtaagaattaaatgaaccaaagaagaattaatgtattcatcaagaagtcatggaggtgaaattggccttgaatgataccactaaggagagaatgttgagatccttatatttagtcaattgtttttaaatctgtagttattaaccacattttaatcatattgaaagggaaattttctgtgatgcatgtattttcaatataaattttagaaaagaagacaattataacttgattttgtgaattacatggaactaaagaaatgacagatttacatttgaaaattgactgaactaaagtacataaataaaagtcatacagaaaaatgtgggaggtgcttgtccatttataaaggacaaaaatgccatttgttgcctaatcattatttcttattggtcagaccaataagaaatcaagagctttgactttaaaggtaagaaaatcttaccttaaaatccccaactgaagggactgtttaaactgtcaactgcagaaaacaagttatggaagttcaggtttagggaaactataaacacaccataacattgagtttatgtgcatagtttgttttatgtacagtgagagtaaattgttagtattatcatgagttgttttgaaacttcaaatttctctagaggggtatgatttaatgttctcaagaggaacataataaaaccatatctggtattagtttttatttttaacaatagcagacttcatacaccaatgttcacagtgtagaccataaaatgcagtcttagtaaaaatattattctctataaagctacaatgagacctccctcaaacatacattgtttttttttttctaacttatgtttggatatatcatcatgatgaactatgttaaaaacaatcagagcttagtaatactttcatattgcttttttattccag

SEQ ID NO: 76 - DNA, 25 bp, Artificial
  Antisense exon 23 skipping inducing oligo
  misc_feature (1)..(25): exon 23 skipping inducing oligonucleotide
aacctcggcttacctgaaattttcg

SEQ ID NO: 77 - DNA, 1653 bp, Hotaria parvula
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 78 - DNA, 17578 bp, Homo sapiens
  Intron (1)..(13645): intron 9
  exon (13646)..(13738): intron 9
  Intron (13739)..(17578): intron 10
gtgagagtggctggctgcgcgtggaggtgtggggggctgcgcctggaggggtagggctgtgcctggaagggtagggctgcgcctggaggtgcgcggttgagcgtggagtcgtgggactgtgcatggaggtgtggggctccccgcacctgagcacccccgcataacaccccagtcccctctggaccctcttcaaggaagttcagttctttattgggctctccactacactgtgagtgccctcctcaggcgagagaacgttctggctcttctcttgccccttcagcccctgttaatcggacagagatggcagggctgtgtctccacggccggaggctctcatagtcagggcacccacagcggttccccacctgccttctgggcagaatacactgccacccataggtcagcatctccactcgtgggccatctgcttaggttgggttcctctggattctggggagattgggggttctgttttgatcagctgattcttctgggagcaagtgggtgctcgcgagctctccagcttcctaaaggtggagaagcacagacttcgggggcctggcctggatccctttccccattcctgtccctgtgcccctcgtctgggtgcgttagggctgacatacaaagcaccacagtgaaagaacagcagtatgcctcctcactagccaggtgtgggcgggtgggtttcttccaaggcctctctgtggccgtgggtagccacctctgtcctgcaccgctgcagtcttccctctgtgtgtgctcctggtagctctgcgcatgctcatcttcttataagaacaccatggcagctgggcgtagtggctcacgcctataatcccagcactttgggaggctgaggcaggcagatcacgaggtcaggagttcgagaccaacctgaccaacagggtgaaacctcgtctctactaaaaatacaaaaatacctgggcgtggtggtggtgcgcgcctataatcccagctactcaggaggctgaggcaggagaatcgcttgaacccaggaggcagaggttgcagtgagccgagatagtgccactgcactccagtttgagcaacagagcgagactctgtctcaaaacaaaataaaacaaaccaaaaaaacccaccatggcttagggcccagcctgatgacctcatttttcacttagtcacctctctaaaggccctgtctccaaatagagtcacattctaaggtacgggggtgttggggaggggggttagggcttcaacatgtgaatttgcggggaccacaattcagcccaggaccccgctcccgccacccagcactggggagctggggaagggtgaagaggaggctgggggtgagaaggaccacagctcactctgaggctgcagatgtgctgggccttctgggcactgggcctcggggagctagggggctttctggaaccctgggcctgcgtgtcagcttgcctcccccacgcaggcgctctccacaccattgaagttcttatcacttgggtctgagcctggggcatttggacggagggtggccaccagtgcacatgggcaccttgcctcaaaccctgccacctccccccacccaggatcccccctgcccccgaacaagcttgtgagtgcagtgtcacatcccatcgggatggaaatggacggtcgggttaaaagggacgcatgtgtagaccctgcctctgtgcatcaggcctcttttgagagtccctgcgtgccaggcggtgcacagaggtggagaagactcggctgtgccccagagcacctcctctcatcgaggaaaggacagacagtggctcccctgtggctgtggggacaagggcagagctccctggaacacaggagggagggaaggaagagaacatctcagaatctccctcctgatggcaaacgatccgggttaaattaaggtccggccttttcctgctcaggcatgtggagcttgtagtggaagaggctctctggaccctcatccaccacagtggcctggttagagaccttggggaaataactcacaggtgacccagggcctctgtcctgtaccgcagctgagggaaactgtcctgcgcttccactggggacaatgcgctccctcgtctccagactttccagtcctcattcggttctcgaaagtcgcctccagaagccccatcttgggaccaccgtgactttcattctccagggtgcctggccttggtgctgcccaagaccccagaggggccctcactggcctttcctgccttttctcccattgcccacccatgcacccccatcctgctccagcacccagactgccatccaggatctcctcaagtcacataacaagcagcacccacaaggtgctcccttccccctagcctgaatctgctgctccccgtctggggttccccgcccatgcacctctgggggcccctgggttctgccataccctgccctgtgtcccatggtggggaatgtccttctctccttatctcttcccttcccttaaatccaagttcagttgccatctcctccaggaagtcttcctggattcccctctctcttcttaaagcccctgtaaactctgaccacactgagcatgtgtctgctgctccctagtctgggccatgagtgagggtggaggccaagtctcatgcatttttgcagcccccacaagactgtgcaggtggccggccctcattgaatgcggggttaatttaactcagcctctgtgtgagtggatgattcaggttgccagagacagaaccctcagcttagcatgggaagtagcttccctgttgaccctgagttcatctgaggttggcttggaaggtgtgggcaccatttggcccagttcttacagctctgaagagagcagcaggaatggggctgagcagggaagacaactttccattgaaggcccctttcagggccagaactgtccctcccaccctgcagctgccctgcctctgcccatgaggggtgagagtcaggcgacctcatgccaagtgtagaaaggggcagacgggagccccaggttatgacgtcaccatgctgggtggaggcagcacgtccaaatctactaaagggttaaaggagaaagggtgacttgacttttcttgagatattttgggggacgaagtgtggaaaagtggcagaggacacagtcacagcctcccttaaatgccaggaaagcctagaaaaattgtctgaaactaaacctcagccataacaaagaccaacacatgaatctccaggaaaaaagaaaaagaaaaatgtcatacagggtccatgcacaagagcctttaaaatgacccgctgaagggtgtcaggcctcctcctcctggactggcctgaaggctccacgagcttttgctgagacctttgggtccctgtggcctcatgtagtacccagtatgcagtaagtgctcaataaatgtttggctacaaaagaggcaaagctggcggagtctgaagaatccctcaaccgtgccggaacagatgctaacaccaaagggaaaagagcaggagccaagtcacgtttgggaacctgcagaggctgaaaactgccgcagattgctgcaaatcattgggggaaaaacggaaaacgtctgttttcccctttgtgcttttctctgttttcttctttgtgcttttctctgttttcaggatttgctacagtgaacatagattgctttggggccccaaatggaattattttgaaaggaaaatgcagataatcaggtggccgcactggagcaccagctgggtaggggtagagattgcaggcaaggaggaggagctgggtggggtgccaggcaggaagagcccgtaggccccgccgatcttgtgggagtcgtgggtggcagtgttccctccagactgtaaaagggagcacctggcgggaagagggaattcttttaaacatcattccagtgcccgagcctcctggacctgttgtcatcttgaggtgggcctcccctgggtgactctagtgtgcagcctggctgagactcagtggccctgggttcttactgctgacacctaccctcaacctcaaccactgcggcctcctgtgcaccctgatccagtggctcattttccactttcagtcccagctctatccctatttgcagtttccaagtgcctggtcctcagtcagctcagacccagccaggccagcccctggttcccacatcccctttgccaagctcatccccgccctgtttggcctgcgggagtgggagtgtgtccagacacagagacaaaggaccagcttttaaaacattttgttggggccaggtgtggtggctcacacctaatcccaacacctggggaggccaaggcagaaggatcacttgagtccaggagttcaagaccagcctgggcaacatagggagaccctgtctctacaattttttttttaattagctgggcctgttggcactctcctgtagttccagctactctagaggctgaggtgggaggactgcttgagcctgggaggtcagggctgcaatgagccatgttcacaccactgaacgccagcctgggcgagaccctgtatcaaaaaagtaaagtaaaatgaatcctgtacgttatattaaggtgccccaaattgtacttagaaggatttcatagttttaaatacttttgttatttaaaaaattaaatgactgcagcatataaattaggttcttaatggaggggaaaaagagtacaagaaaagaaataagaatctagaaacaaagataagagcagaaataaaccagaaaacacaaccttgcactcctaacttaaaaaaaaaaatgaagaaaacacaaccagtaaaacaacatataacagcattaagagctggctcctggctgggcgcggtggcgcatgcctgtaatcccaacactttgggaggccgatgctggaggatcacttgagaccaggagttcaaggttgcagtgagctatgatcataccactacaccctagcctgggcaacacagtgagactgagactctattaaaaaaaaaatgctggttccttccttatttcattcctttattcattcattcagacaacatttatggggcacttctgagcaccaggctctgtgctaagagcttttgcccccagggtccaggccaggggacaggggcaggtgagcagagaaacagggccagtcacagcagcaggaggaatgtaggatggagagcttggccaggcaaggacatgcagggggagcagcctgcacaagtcagcaagccagagaagacaggcagacccttgtttgggacctgttcagtggcctttgaaaggacagcccccacccggagtgctgggtgcaggagctgaaggaggatagtggaacactgcaacgtggagctcttcagagcaaaagcaaaataaacaactggaggcagctggggcagcagagggtgtgtgttcagcactaaggggtgtgaagcttgagcgctaggagagttcacactggcagaagagaggttggggcagctgcaagcctctggacatcgcccgacaggacagagggtggtggacggtggccctgaagagaggctcagttcagctggcagtggccgtgggagtgctgaagcaggcaggctgtcggcatctgctggggacggttaagcaggggtgagggcccagcctcagcagcccttcttggggggtcgctgggaaacatagaggagaactgaagaagcagggagtcccagggtccatgcagggcgagagagaagttgctcatgtggggcccaggctgcaggatcaggagaactggggaccctgtgactgccagcggggagaagggggtgtgcaggatcatgcccagggaagggcccaggggcccaagcatgggggggcctggttggctctgagaagatggagctaaagtcactttctcggaggatgtccaggccaatagttgggatgtgaagacgtgaagcagcacagagcctggaagcccaggatggacagaaacctacctgagcagtggggctttgaaagccttggggcggggggtgcaatattcaagatggccacaagatggcaatagaatgctgtaactttcttggttctgggccgcagcctgggtggctgcttccttccctgtgtgtattgatttgtttctcttttttgagacagagtcttgctgggttgcccaggctggagtgcagtggtgcgatcatagctcactgcagccttgaagtcctgagctcaagagatccttccacctcagcctcctgagtagttgggaccacaggcttgcaccacagtgcccaactaatttcttatattttttgtagagatggggtttcactgtgtcgcccaggatggtcttgaactcctgggctcaagtgatcctcctgcctcagcctcgcaaattgctgggattacaggtgtgagccaccatgcccgaccttctctttttaagggcgtgtgtgtgtgtgtgtgtgtgtgggcgcactctcgtcttcaccttcccccagccttgctctgtctctacccagtcacctctgcccatctctccgatctgtttctctctccttttacccctctttcctccctcctcatacaccactgaccattatagagaactgagtattctaaaaatacattttatttatttattttgagacagagtctcactctgtcacccaggctggagtgcagtggtgcaatctcggctcactgcaacctccgcctcccaggttgaagcaactctcctgcctcagcctccctagtagctgggattacaagcacacaccaccatgcctagcaaatttttatatttttagtagaggaggagtgtcaccatgtttgccaagctggtctcaaactcctggcctcaggtgatctgcctaccttggtctcccaaagtgctgggattacaggtgtgagccaccacgcctgcccttaaaaatacattatatttaatagcaaagccccagttgtcactttaaaaagcatctatgtagaacatttatgtggaataaatacagtgaatttgtacgtggaatcgtttgcctctcctcaatcagggccagggatgcaggtgagcttgggctgagatgtcagaccccacagtaagtggggggcagagccaggctgggaccctcctctaggacagctctgtaactctgagaccctccaggcatcttttcctgtacctcagtgcttctgaaaaatctgtgtgaatcaaatcattttaaaggagcttgggttcatcactgtttaaaggacagtgtaaataattctgaaggtgactctaccctgttatttgatctcttctttggccagctgacttaacaggacatagacaggttttcctgtgtcagttcctaagctgatcaccttggacttgaagaggaggcttgtgtgggcatccagtgcccaccccgggttaaactcccagcagagtattgcactgggcttgctgagcctggtgaggcaaagcacagcacagcgagcaccaggcagtgctggagacaggccaagtctgggccagcctgggagccaactgtgaggcacggacggggctgtggggctgtggggctgcaggcttggggccagggagggagggctgggctctttggaacagccttgagagaactgaacccaaacaaaaccagatcaaggtctagtgagagcttagggctgctttgggtgctccaggaaattgattaaaccaagtggacacacacccccagccccacctcaccacagcctctccttcagggtcaaactctgaccacagacatttctcccctgactaggagttccctggatcaaaattgggagcttgcaacacatcgttctctcccttgatggtttttgtcagtgtctatccagagctgaagtgtaatatatatgttactgtagctgagaaattaaatttcaggattctgatttcataatgacaaccattcctcttttctctcccttctgtaaatctaagattctataaacggtgttgacttaatgtgacaattggcagtagttcaggtctgctttgtaaatacccttgtgtctattgtaaaatctcacaaaggcttgttgccttttttgtggggttagaacaagaaaaagccacatggaaaaaaaatttcttttttgtttttttgtttgcttgtttttttgagacagagtttcactctgtcgcccaggctggagtgcagtggtgcgatctccgcccactgcaagctccacctcccgggttcatgctattctcctgtctcagcctcccaagtagctgggactgcaggtgcccgccaccacacctggctaatttttttgtatttttagtagagacggggtttcaccgtgttagccaggatggtctcaatctcctgacctcgtcatctgcctgcctcggcctcccaaagtgctgagattacaggcgtgagccaccgtgcccggccagaaaaaaacatttctaagtatgtggcagatactgaattattgcttaatgtcctttgattcatttgtttaatttctttaatggattagtacagaaaacaaagttctcttccttgaaaaactggtaagttttctttgtcagataaggagagttaaataacccatgacatttccctttttgcctcggcttccaggaagctcaaagttaaatgtaatgatcactcttgtaattatcagtgttgatgcccttcccttcttctaatgttactctttacattttcctgctttattattgtgtgtgttttctaattctaagctgttcccactcctttctgaaagcaggcaaatcttctaagccttatccactgaaaagttatgaataaaaaatgatcgtcaagcctacaggtgctgaggctactccagaggctgaggccagaggaccacttgagcccaggaatttgagacctgggctgggcagcatagcaagactctatctccattaaaactatttttttttatttaaaaaataatccgcaaagaaggagtttatgtgggattccttaaaatcggagggtggcatgaattgattcaaagacttgtgcagagggcgacagtgactccttgagaagcagtgtgagaaagcctgtcccacctccttccgcagctccagcctgggctgaggcactgtcacagtgtctccttgctggcaggagagaatttcaacattcaccaaaaagtagtattgtttttattaggtttatgaggctgtagccttgaggacagcccaggacaactttgttgtcacatagatagcctgtggctacaaactctgagatctagattcttctgcggctgcttctgacctgagaaagttgcggaacctcagcgagcctcacatggcctccttgtccttaacgtggggacggtgggcaagaaaggtgatgtggcactagagatttatccatctctaaaggaggagtggattgtacattgaaacaccagagaaggaattacaaaggaagaatttgagtatctaaaaatgtaggtcaggcgctcctgtgttgattgcagggctattcacaatagccaagatttggaagcaacccaagtgtccatcaacagacaaatggataaagaaaatgtggtgcatatacacaatggaatactattcagccatgaaaaagaatgagaatctgtcatttgaaacaacatggatggaactggaggacattatgttaagtgaaataagccagacagaaggacagacttcacatgttctcacacatttgtgggagctaaaaattaaactcatggagatagagagtagaaggatggttaccagaggctgaggagggtggaggggagcagggagaaagtagggatggttaatgggtacaaaaacgtagttagcatgcatagatctagtattggatagcacagcagggtgacgacagccaacagtaatttatagtacatttaaaaacaactaaaagagtgtaactggactggctaacatggtgaaaccccgtctctactaaaaatacaaaaattagctgggcacggtggctcacgcctgtaatcccagcactttgggaggccgaggcgggccgatcacgaggtcaggagatcgagaccatcctagctaacatggtgaaaccccgtctctactacaaatacaaaaaaaagaaaaaattagccgggcatggtggtgggcgcctgtagtcccagctactcgggaggctgaggcaggagaatggcgtgaacccgggaggcggagcttgcagtgagccgagatcgcgccactgcactccagcctgggcgacaaggcaagattctatctcaaaaaaataaaaataaaataaaataaaataataaaataaaataaaataaaataaaataaaataaataaaataaaatgtataattggaatgtttataacacaagaaatgataaatgcttgaggtgatagataccccattcaccgtgatgtgattattgcacaatgtatgtctgtatctaaatatctcatgtaccccacaagtatatacacctactatgtacccatataaatttaaaattaaaaaattataaaacaaaaataaataagtaaattaaaatgtaggctggacaccgtggttcacgcctgtaatcccagtgctttgtgaggctgaggtgagagaatcacttgagcccaggagtttgagaccggcctgggtgacatagcgagaccccatcatcacaaagaatttttaaaaattagctgggcgtggtagcacataccggtagttccagctacttgggagaccgaggcaggaggattgcttgagcccaggagtttaaggctgcagtgagctacgatggcgccactgcattccagcctgggtgacagagtgagagcttgtctctattttaaaaataataaaaagaataaataaaaataaattaaaatgtaaatatgtgcatgttagaaaaaatacacccatcagcaaaaagggggtaaaggagcgatttcagtcataattggagagatgcagaataagccagcaatgcagtttcttttattttggtcaaaaaaaataagcaaaacaatgttgtaaacacccagtgctggcagcaatgtggtgaggctggctctctcaccagggctcacagggaaaactcatgcaacccttttagaaagccatgtggagagttgtaccgagaggttttagaatatttataactttgacccagaaattctattctaggactctgtgttatgaaaataacccatcatatggaaaaagctcctttcagaaagaggttcatgggaggctgtttgtattttttttttctttgcatcaaatccagctcctgcaggactgtttgtattattgaagtacaaagtggaatcaatacaaatgttggatagcaggggaacaatattcacaaaatggaatgggacatagtattaaacatagtgcttctgatgaccgtagaccatagacaatgcttaggatatgatatcacttcttttgttgttttttgtattttgagacgaagtctcattctgtcacccaggctggagttcagtggcgccatctcagctcactgcaacctccatctcccgggttcaagctattctccttcctcaacctcccgagtagctgggttgcgcaccaccatgcctggctaacttttgtatttttagtacagacggggtttcaccacgttggccaggctgctcttgaactcctgacgtcaggtgatccaccagccttgacctcccaaagtgctaggattacaggagccactgtacccagcctaggatatgatatcacttcttagagcaagatacaaaattgcatgtgcacaataattctaccaagtataggtatacaggggtagttatatataaatgagacttcaaggaaatacaacaaaatgcaatcgtgattgtgttagggtggtaagaaaacggtttttgctttgatgagctctgttttttaaaatcgttatattttctaataaaaatacatagtcttttgaaggaacataaaagattatgaagaaatgagttagatattgattcctattgaagattcagacaagtaaaattaaggggaaaaaaaacgggatgaaccagaagtcaggctggagttccaaccccagatccgacagcccaggctgatggggcctccagggcagtggtttccacccagcattctcaaaagagccactgaggtctcagtgccattttcaagatttcggaagcggcctgggcacggctggtccttcactgggatcaccacttggcaattatttacacctgagacgaatgaaaaccagagtgctgagattacaggcatggtggcttacgcttgtaatcggctttgggaagccgaggtgggctgattgcttgagcccaggagtttcaaactatcctggacaacatagcatgacctcgtctctacaaaaaatacaaaaaatttgccaggtgtggtggcatgtgcctgtggtcccagctacttgggaggctgaagtaggagaatcccctgagccctgggaagtcgaggctgcactgagccgtgatggtgtcactgcactccagcctgggtgacaaagtgagaccctatctcacaaagaaaaaaaacaaaacaaaaaacccaaagcacactgtttccactgtttccagagttcctgagaggaaaggtcaccgggtgaggaagacgttctcactgatctggcagagaaaatgtccagtttttccaactccctaaaccatggttttctatttcatagttcttaggcaaattggtaaaaatcatttctcatcaaaacgctgatattttcacacctccctggtgtctgcagaaagaaccttccagaaatgcagtcgtgggagacccatccaggccacccctgcttatggaagagctgagaaaaagccccacgggagcatttgctcagcttccgttacgcacctagtggcattgtgggtgggagagggctggtgggtggatggaaggagaaggcacagcccccccttgcagggacagagccctcgtacagaagggacaccccacatttgtcttccccacaaagcggcctgtgtcctgcctacggggtcagggcttctcaaacctggctgtgtgtcagaatcaccaggggaacttttcaaaactagagagactgaagccagactcctagattctaattctaggtcagggctaggggctgagattgtaaaaatccacaggtgattctgatgcccggcaggcttgagaacagccgcagggagttctctgggaatgtgccggtgggtctagccaggtgtgagtggagatgccggggaacttcctattactcactcgtcagtgtggccgaacacatttttcacttgacctcaggctggtgaacgctcccctctggggttcaggcctcacgatgccatccttttgtgaagtgaggacctgcaatcccagcttcgtaaagcccgctggaaatcactcacacttctgggatgccttcagagcagccctctatcccttcagctcccctgggatgtgactcgacctcccgtcactccccagactgcctctgccaagtccgaaagtggaggcatccttgcgagcaagtaggcgggtccagggtggcgcatgtcactcatcgaaagtggaggcgtccttgcgagcaagcaggcgggtccagggtggcgtgtcactcatccttttttctggctaccaaaggtgcagataattaataagaagctggatcttagcaacgtccagtccaagtgtggctcaaaggataatatcaaacacgtcccgggaggcggcagtgtgagtaccttcacacgtcccatgcgccgtgctgtggcttgaattattaggaagtggtgtgagtgcgtacacttgcgagacactgcatagaataaatccttcttgggctctcaggatctggctgcgacctctgggtgaatgtagcccggctccccacattcccccacacggtccactgttcccagaagccccttcctcatattctaggagggggtgtcccagcatttctgggtcccccagcctgcgcaggctgtgtggacagaatagggcagatgacggaccctctctccggaccctgcctgggaagctgagaatacccatcaaagtctccttccactcatgcccagccctgtccccaggagccccatagcccattggaagttgggctgaaggtggtggcacctgagactgggctgccgcctcctcccccgacacctgggcaggttgacgttgagtggctccactgtggacaggtgacccgtttgttctgatgagcggacaccaaggtcttactgtcctgctcagctgctgctcctacacgttcaaggcaggagccgattcctaagcctccagcttatgcttagcctgcgccaccctctggcagagactccagatgcaaagagccaaaccaaagtgcgacaggtccctctgcccagcgttgaggtgtggcagagaaatgctgcttttggcccttttagatttggctgcctcttgccaggagtggtggctcgtgcctgtaattccagcactttgggagactaaggcgggaggttcgcttgagcccaggagttcaagaccagcctgggcaacaatgagacccctgtgtctacaaaaagaattaaaattagccaggtgtggtggcacgcacctgtagtcccagctacttgggaggctgaggtgggaggattgcctgagtccgggaggcggaagttgcaaggagccatgatcgcgccactgcacttcaacctaggcaacagagtgagactttgtctcaaaaaacaatcatataataattttaaaataaatagatttggcttcctctaaatgtccccggggactccgtgcatcttctgtggagtgtctccgtgagattcgggactcagatcctcaagtgcaactgacccacccgataagctgaggcttcatcatcccctggccggtctatgtcgactgggcacccgaggctcctctcccaccagctctcttggtcagctgaaagcaaactgttaacaccctggggagctggacgtatgagacccttggggtgggaggcgttgatttttgagagcaatcacctggccctggctggcagtaccgggacactgctgtggctccggggtgggctgtctccagaaaatgcctggcctgaggcagccacccgcatccagcccagagggtttattcttgcaatgtgctgctgcttcctgccctgagcacctggatcccggcttctgccctgaggccccttgagtcccacaggtagcaagcgcttgccctgcggctgctgcatggggctaactaacgcttcctcaccagtgtctgctaagtgtctcctctgtctcccacgccctgctctcctgtccccccagtttgtctgctgtgaggggacagaagaggtgtgtgccgcccccacccctgcccgggcccttgttcctgggattgctgttttcagctgtttgagctttgatcctggttctctggcttcctcaaagtgagctcggccagaggaggaaggccatgtgctttctggttgaagtcaagtctggtgccctggtggaggctgtgctgctgaggcggagctggggagagagtgcacacgggctgcgtggccaacccctctgggtagctgatgcccaaagacgctgcagtgcccaggacatctgggacctccctggggcccgcccgtgtgtcccgcgctgtgttcatctgcgggctagcctgtgacccgcgctgtgctcgtctgcgggctagcctgtgtcccgcgctctgcttgtctgcggtctagcctgtgacctggcagagagccaccagatgtcccgggctgagcactgccctctgagcaccttcacaggaagcccttctcctggtgagaagagatgccagcccctggcatctgggggcactggatccctggcctgagccctagcctctccccagcctgggggccccttcccagcaggctggccctgctccttctctacctgggacccttctgcctcctggctggaccctggaagctctgcagggcctgctgtccccctccctgccctccaggtatcctgaccaccggccctggctcccactgccatccactcctctcctttctggccgttccctggtccctgtcccagcccccctccccctctcacgagttacctcacccaggccagagggaagagggaaggaggccctggtcataccagcacgtcctcccacctccctcggccctggtccaccccctcagtgctggcctcagagcacagctctctccaagccaggccgcgcgccatccatcctccctgtcccccaacgtccttgccacagatcatgtccgccctgacacacatgggtctcagccatctctgccccagttaactccccatccataaagagcacatgccagccgacaccaaaataattcgggatggttccagtttagacctaagtggaaggagaaaccaccacctgccctgcaccttgttttttggtgaccttgataaaccatcttcagccatgaagccagctgtctcccaggaagctccagggcggtgcttcctcgggagctgactgataggtgggaggtggctgcccccttgcaccctcaggtgaccccacacaaggccactgctggaggccctggggactccaggaatgtcaatcagtgacctgccccccaggccccacacagccatggctgcatagaggcctgcctccaagggacctgtctgtctgccactgtggagtccctacagcgtgccccccacaggggagctggttctttgactgagatcagctggcagctcagggtcatcattcccagagggagcggtgccctggaggccacaggcctcctcatgtgtgtctgcgtccgctcgagcttactgagacactaaatctgttggtttctgctgtgccacctacccaccctgttggtgttgctttgttcctattgctaaagacaggaatgtccaggacactgagtgtgcaggtgcctgctggttctcacgtccgagctgctgaactccgctgggtcctgcttactgatggtctttgctctagtgctttccagggtccgtggaagcttttcctggaataaagcccacgcatcgaccctcacagcgcctcccctctttgaggcccagcagataccccactcctgcctttccagcaagatttttcagatgctgtgcatactcatcatattgatcacttttttcttcatgcctgattgtgatctgtcaatttcatgtcaggaaagggagtgacatttttacacttaagcgtttgctgagcaaatgtctgggtcttgcacaatgacaatgggtccctgtttttcccagaggctcttttgttctgcagggattgaagacactccagtcccacagtccccagctcccctggggcagggttggcagaatttcgacaacacatttttccaccctgactaggatgtgctcctcatggcagctgggaaccactgtccaataagggcctgggcttacacagctgcttctcattgagttacacccttaataaaataatcccattttatcctttttgtctctctgtcttcctctctctctgcctttcctcttctctctcctcctctctcatctccag

SEQ ID NO: 79 - DNA, 18 bp, Artificial
  Synthetic oligonucleotide
tatctgcacctttggtag

SEQ ID NO: 80 - DNA, 21 bp, Artificial
  Synthetic oligonucleotide
tgaaggtactcacactgccgc

SEQ ID NO: 81 - DNA, 20 bp, Artificial
  guide RNA
tgcaaaaacccaaaatattt

SEQ ID NO: 82 - DNA, 20 bp, Artificial
  guide RNA
aaaatattttagctcctact

SEQ ID NO: 83 - DNA, 20 bp, Artificial
  guide RNA
cagagtaacagtctgagtag

SEQ ID NO: 84 - DNA, 20 bp, Artificial
  guide RNA
taagggatatttgttcttac

SEQ ID NO: 85 - DNA, 20 bp, Artificial
  guide RNA
ctaagggatatttgttctta

SEQ ID NO: 86 - DNA, 20 bp, Artificial
  guide RNA
tgttcttacaggcaacaatg

SEQ ID NO: 87 - DNA, 20 bp, Artificial
  guide RNA
tgtatgcttttctgttaaag

SEQ ID NO: 88 - DNA, 20 bp, Artificial
  guide RNA
atgtgtatgcttttctgtta

SEQ ID NO: 89 - DNA, 20 bp, Artificial
  guide RNA
gtgtatgcttttctgttaaa

SEQ ID NO: 90 - DNA, 20 bp, Artificial
  guide RNA
ttgcctttttggtatcttac

SEQ ID NO: 91 - DNA, 20 bp, Artificial
  guide RNA
tttgcctttttggtatctta

SEQ ID NO: 92 - DNA, 20 bp, Artificial
  guide RNA
cgctgcccaatgccatcctg

SEQ ID NO: 93 - DNA, 20 bp, Artificial
  guide RNA
atttatttttccttttattc

SEQ ID NO: 94 - DNA, 20 bp, Artificial
  guide RNA
tttccttttattctagttga

SEQ ID NO: 95 - DNA, 20 bp, Artificial
  guide RNA
tgattctgaattctttcaac

SEQ ID NO: 96 - DNA, 20 bp, Artificial
  guide RNA
atccatatgcttttacctgc

SEQ ID NO: 97 - DNA, 20 bp, Artificial
  guide RNA
gatccatatgcttttacctg

SEQ ID NO: 98 - DNA, 20 bp, Artificial
  guide RNA
cagatctgtcaaatcgcctg

SEQ ID NO: 99 - DNA, 20 bp, Artificial
  guide RNA
ttattcttctttctccaggc

SEQ ID NO: 100 - DNA, 20 bp, Artificial
  guide RNA
aattttattcttctttctcc

SEQ ID NO: 101 - DNA, 20 bp, Artificial
  guide RNA
caattttattcttctttctc

SEQ ID NO: 102 - DNA, 20 bp, Artificial
  guide RNA
gttttaaaatttttatatta

SEQ ID NO: 103 - DNA, 20 bp, Artificial
  guide RNA
ttttatattacagaatataa

SEQ ID NO: 104 - DNA, 20 bp, Artificial
  guide RNA
atattacagaatataaaaga

SEQ ID NO: 105 - DNA, 20 bp, Artificial
  guide RNA
tgtgtatgtgtatgtgtttt

SEQ ID NO: 106 - DNA, 20 bp, Artificial
  guide RNA
tatgtgtatgtgttttaggc

SEQ ID NO: 107 - DNA, 20 bp, Artificial
  guide RNA
ctattccagtcaaataggtc

SEQ ID NO: 108 - DNA, 20 bp, Artificial
  guide RNA
gtgtagtgttaatgtgctta

SEQ ID NO: 109 - DNA, 20 bp, Artificial
  guide RNA
ggacttcttatctggatagg

SEQ ID NO: 110 - DNA, 20 bp, Artificial
  guide RNA
taggtggtatcaacatctgt

SEQ ID NO: 111 - DNA, 20 bp, Artificial
  guide RNA
tgaaaatttatttccacatg

SEQ ID NO: 112 - DNA, 20 bp, Artificial
  guide RNA
gaaaatttatttccacatgt

SEQ ID NO: 113 - DNA, 20 bp, Artificial
  guide RNA
ttacatttttgacctacatg

SEQ ID NO: 114 - DNA, 20 bp, Artificial
  guide RNA
aaagaaaatcacagaaacca

SEQ ID NO: 115 - DNA, 20 bp, Artificial
  guide RNA
aaaatcacagaaaccaaggt

SEQ ID NO: 116 - DNA, 20 bp, Artificial
  guide RNA
ggtatctttgatactaacct

SEQ ID NO: 117 - DNA, 20 bp, Artificial
  guide RNA
tatgtgttacctacccttgt

SEQ ID NO: 118 - DNA, 20 bp, Artificial
  guide RNA
aaatgtacaaggaccgacaa

SEQ ID NO: 119 - DNA, 20 bp, Artificial
  guide RNA
gtacaaggaccgacaagggt

SEQ ID NO: 120 - DNA, 20 bp, Artificial
  guide RNA
tgcactattctcaacaggta

SEQ ID NO: 121 - DNA, 20 bp, Artificial
  guide RNA
tcaaatgcactattctcaac

SEQ ID NO: 122 - DNA, 20 bp, Artificial
  guide RNA
ctttacacactttacctgtt

SEQ ID NO: 123 - DNA, 20 bp, Artificial
  guide RNA
atgctctcatccatagtcat

SEQ ID NO: 124 - DNA, 20 bp, Artificial
  guide RNA
tctcatccatagtcataggt

SEQ ID NO: 125 - DNA, 20 bp, Artificial
  guide RNA
catccatagtcataggtaag

SEQ ID NO: 126 - DNA, 20 bp, Artificial
  guide RNA
tgaacatttggtcctttgca

SEQ ID NO: 127 - DNA, 20 bp, Artificial
  guide RNA
tctgaacatttggtcctttg

SEQ ID NO: 128 - DNA, 20 bp, Artificial
  guide RNA
tctcgctcactcaccctgca

SEQ ID NO: 129 - DNA, 20 bp, Artificial
  guide RNA
ggcacagcaatagatctccg

SEQ ID NO: 130 - DNA, 20 bp, Artificial
  guide RNA
taagaactctgaatgtccgc

SEQ ID NO: 131 - DNA, 20 bp, Artificial
  guide RNA
gttcttctgatcaggttgaa

SEQ ID NO: 132 - DNA, 20 bp, Artificial
  guide RNA
tcacgtacctgagagatcct

SEQ ID NO: 133 - DNA, 20 bp, Artificial
  guide RNA
gaatagccacagggcccgag

SEQ ID NO: 134 - DNA, 20 bp, Artificial
  guide RNA
tgaagccttgataaagatac

SEQ ID NO: 135 - DNA, 20 bp, Artificial
  guide RNA
cagatatgagggtgggagaa

SEQ ID NO: 136 - DNA, 20 bp, Artificial
  guide RNA
caggggaatgggttcctggg

SEQ ID NO: 137 - DNA, 20 bp, Artificial
  guide RNA
cccctccctgaactcacact

SEQ ID NO: 138 - DNA, 16 bp, Artificial
  regulatory sequence-binding oligonucleotide
gtactcacctgccctc

SEQ ID NO: 139 - DNA, 16 bp, Artificial
  regulatory sequence-binding oligonucleotide
gaacttacctcggcac

SEQ ID NO: 140 - DNA, 16 bp, Artificial
  regulatory sequence-binding oligonucleotide
ggactcacctagtcag

SEQ ID NO: 141 - DNA, 16 bp, Artificial
  regulatory sequence-binding oligonucleotide
gcacttacctattggc

SEQ ID NO: 142 - DNA, 16 bp, Artificial
  regulatory sequence-binding oligonucleotide
gctattaccttaaccc

SEQ ID NO: 143 - DNA, 247 bp, Artificial
  regulatory sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttctgagggcaggtgagtacaatatttctgcatataaatatttagtccaagctaggcccttttgctaatcatgttcatacctcttatcctcctcccacag

SEQ ID NO: 144 - DNA, 247 bp, Artificial
  regulatory sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttctgtgccgaggtaagttcaatatttctgcatataaatatttagtccaagctaggcccttttgctaatcatgttcatacctcttatcctcctcccacag

SEQ ID NO: 145 - DNA, 247 bp, Artificial
  regulatory sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttctctgactaggtgagtccaatatttctgcatataaatatttagtccaagctaggcccttttgctaatcatgttcatacctcttatcctcctcccacag

SEQ ID NO: 146 - DNA, 247 bp, Artificial
  regulatory sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttctgccaataggtaagtgcaatatttctgcatataaatatttagtccaagctaggcccttttgctaatcatgttcatacctcttatcctcctcccacag

SEQ ID NO: 147 - DNA, 247 bp, Artificial
  regulatory sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttagtccaagctaggcccttttgctaatcatgttcatacctcttatcctcctcccacag

SEQ ID NO: 148 - DNA, 247 bp, Artificial
  regulatory sequence
gtgagtctatgggacccttgatgttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttagtccaagctaggcccttttgctaatcatgttcatacctcttatcctcctcccacag

SEQ ID NO: 149 - DNA, 16 bp, Artificial
  regulatory sequence-binding oligonucleotide
gctattgccttaaccc

SEQ ID NO: 150 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 151 - protein, 1307 aa, Acidaminococcus fermentans
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 152 - protein, 984 aa, Campylobacter jejuni
MARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 153 - protein, 9 aa, Artificial
  structural motif
LAGLIDADG

SEQ ID NO: 154 - protein, 887 aa, Natronobacterium gregoryi
MTVIDLDSTTTADELTSGHTYDISVTLTGVYDNTDEQHPRMSLAFEQDNGERRYITLWKNTTPKDVFTYDYATGSTYIFTNIDYEVKDGYENLTATYQTTVENATAQEVGTTDEDETFAGGEPLDHHLDDALNETPDDAETESDSGHVMTSFASRDQLPEWTLHTYTLTATDGAKTDTEYARRTLAYTVRQELYTDHDAAPVATDGLMLLTPEPLGETPLDLDCGVRVEADETRTLDYTTAKDRLLARELVEEGLKRSLWDDYLVRGIDEVLSKEPVLTCDEFDLHERYDLSVEVGHSGRAYLHINFRHRFVPKLTLADIDDDNIYPGLRVKTTYRPRRGHIVWGLRDECATDSLNTLGNQSVVAYHRNNQTPINTDLLDAIEAADRRVVETRRQGHGDDAVSFPQELLAVEPNTHQIKQFASDGFHQQARSKTRLSASRCSEKAQAFAERLDPVRLNGSTVEFSSEFFTGNNEQQLRLLYENGESVLTFRDGARGAHPDETFSKGIVNPPESFEVAVVLPEQQADTCKAQWDTMADLLNQAGAPPTRSETVQYDAFSSPESISLNVAGAIDPSEVDAAFVVLPPDQEGFADLASPTETYDELKKALANMGIYSQMAYFDRFRDAKIFYTRNVALGLLAAAGGVAFTTEHAMPGDADMFIGIDVSRSYPEDGASGQINIAATATAVYKDGTILGHSSTRPQLGEKLQSTDVRDIMKNAILGYQQVTGESPTHIVIHRDGFMNEDLDPATEFLNEQGVEYDIVEIRKQPQTRLLAVSDVQYDTPVKSIAAINQNEPRATVATFGAPEYLATRDGGGLPRPIQIERVAGETDIETLTRQVYLLSQSHIQVHNSTARLPITTAYADQASTHATKGYLVQTGAFESNVGFL